(19) United States
(12) Patent Application Publication
(10) Pub. No.: US 2025/0241757 A1
(43) Pub. Date: Jul. 31, 2025

(54) HEART VALVE REPAIR DEVICES AND METHODS

(71) Applicant: EDWARDS LIFESCIENCES CORPORATION, Irvine, CA (US)

(72) Inventors: Charles Henry Bloodworth, IV, Irvine, CA (US); Amanda Tjipta Sugijoto, Tustin, CA (US); Rhayoung Kwon, Irvine, CA (US); Zachary James Zira, Mission Viejo, CA (US); Lyon Huynh Ly, Anaheim, CA (US); Bryant Quoc Tran, Anaheim, CA (US); Bar Eytan-Vaisman, Gedera (IL); Yaron Herman, Givat Ada (IL); Itay Avinathan, Tel-Aviv (IL)

(21) Appl. No.: 19/184,480

(22) Filed: Apr. 21, 2025

Related U.S. Application Data
(63) Continuation of application No. PCT/US2023/036151, filed on Oct. 27, 2023.
(60) Provisional application No. 63/420,440, filed on Oct. 28, 2022.

(51) Int. Cl. A61F 2/24

(52) U.S. Cl. CPC A61F 2/2463; A61F 2/2466; A61F 2220/0008

(57) ABSTRACT

An implantable device to be positioned within a heart valve, using a delivery system comprising catheters. The device can include leaflet anchors for attaching to valve leaflets. One or more tethers can be slackened to allow the anchors to move relatively apart and/or relatively away from a spacer. The one or more tethers can be pulled to move the anchors closer to one another or and/or closer to the spacer.

### CROSS-REFERENCES TO RELATED APPLICATIONS

The present application is a continuation of Patent Cooperation Treaty Application PCT/US2023/036151, filed on Oct. 27, 2023, and titled “Heart Valve Repair Devices and Methods,” which claims priority to Provisional U.S. Patent Application 63/420,440 to Bloodworth et al., filed Oct. 28, 2022, and titled “Heart Valve Repair Devices and Methods,” which are both incorporated herein by reference for all purposes.

### BACKGROUND

A healthy heart has a generally conical shape that tapers to a lower apex. The heart is four-chambered through which blood flows, A native human heart includes four chambers: two atria (upper chambers)—the left atrium and the right atrium, and two ventricles (lower chambers)—the left ventricle and the right ventricle. A wall, the septum, separates the right and left sides of the heart (i.e., of each pair of chambers). For each of the four chambers, there is a respective native valve (the mitral, tricuspid, aortic, and pulmonary valves, respectively) preventing the upstream (backward) flow into that chamber.

On the right side of the heart, deoxygenated blood from the body is received in the right atrium and passes downstream (forward) through the tricuspid valve to the right ventricle. From the right ventricle it is pumped downstream through the pulmonary valve to the lungs by way of a blood vessel for blood with low oxygen levels, the pulmonary artery. On the left side of the heart, oxygenated blood from the lungs is received in the left atrium and passes downstream through the mitral valve to the left ventricle. From the left ventricle it is pumped downstream through the aortic valve to the remainder of the body by way of a blood vessel for oxygen rich blood, the aorta. During each heartbeat, there is a diastolic phase (cardiac diastole) when the chambers relax and fill passively with blood, and a systolic phase when the heart contracts and pumps blood into the two blood vessels downstream of the ventricles (i.e., the pulmonary artery and the aorta). The systolic phase includes an atrial systole, when the atria contract to eject blood from within the atria downstream into their respective ventricles; and ventricular systole, when the ventricles contract to eject blood from within the ventricles downstream into their respective downstream blood vessels.

Structurally, the tricuspid, aortic, and pulmonary heart valves have three leaflets, while the mitral valve has two leaflets (also known as cusps). In each case, the leaflets operate as flaps that open and close to allow the downstream (forward) flow of blood when pressure urges the blood forward through the valve and obstructs the flow of blood through the valve when pressure urges the blood to flow upstream (backward) through the valve (as occurs for the mitral and tricuspid valves during ventricular systole).

For example, the mitral valve, which connects the left atrium to the left ventricle, includes an annulus portion, which is an annular portion of the valve's tissue surrounding a mitral valve orifice, and a pair of leaflets extending downward from the annulus portion into the left ventricle. The mitral valve annulus portion can form a “D”-shaped, oval, or otherwise out-of-round cross-sectional shape having major and minor axes. An anterior leaflet can be larger than a posterior leaflet, forming a generally “C”-shaped boundary between the abutting sides of the leaflets when they are closed together.

With the mitral valve operating properly, the anterior leaflet and the posterior leaflet of the mitral valve function together as a one-way valve to allow blood to flow only from the left atrium to the left ventricle. During atrial systole, after the left atrium received oxygenated blood from the pulmonary veins during cardiac diastole, the muscles of the left atrium contract and the left ventricle dilates (also referred to as “ventricular diastole” or “diastole”), and the oxygenated blood that is collected in the left atrium flows into the left ventricle. Subsequently, during ventricular systole, the muscles of the left atrium relax and the muscles of the left ventricle contract, and the resulting increased blood pressure in the left ventricle urges the sides of the two leaflets together, thereby closing the one-way mitral valve so that blood cannot flow back to the left atrium. This pressurized blood is instead expelled out of the left ventricle through the aortic valve. To prevent the two leaflets from prolapsing under pressure and folding back through the mitral annulus toward the left atrium, a plurality of fibrous cords called chordae tendineae tether the leaflets to papillary muscles in the left ventricle.

The native heart valves serve critical functions in assuring the forward flow of an adequate supply of blood through the cardiovascular system. In abnormal situations, these heart valves can be dysfunctional (e.g., damaged, or otherwise malformed), and thus less effective, such as by congenital malformations, inflammatory processes, infectious conditions, disease, and the like. Such dysfunctionality can result in serious cardiovascular compromise or death. One form of dysfunctionality is valvular regurgitation.

Valvular regurgitation involves the valve improperly allowing some blood to flow in the wrong direction through the valve. For example, mitral regurgitation occurs when the mitral valve fails to close properly and blood flows upstream into the left atrium from the left ventricle during ventricle systole. Likewise, tricuspid regurgitation occurs when the tricuspid valve fails to close properly and blood flows upstream into the right atrium from the right ventricle during ventricle systole.

Mitral regurgitation is one of the most common forms of valvular heart disease. Mitral regurgitation can have many different causes, such as leaflet prolapse, dysfunctional papillary muscles, stretching of the mitral valve annulus resulting from dilation of the left ventricle, more than one of these, etc. Mitral regurgitation at a central portion of the leaflets can be referred to as central jet mitral regurgitation and mitral regurgitation nearer to one commissure (i.e., location where the leaflets meet) of the leaflets can be referred to as eccentric jet mitral regurgitation. Central jet regurgitation occurs when the edges of the leaflets do not meet in the middle and thus the valve does not close, and regurgitation is present. Regurgitation in any of the three-leaflet valves is likewise a serious form of heart disease having many different causes and being characterized by various forms of valve failure mechanisms.

Prior art FIG. 1 shows a device for repairing leaflets, e.g., an anterior leaflet 20 and a posterior leaflet 22, of a dysfunctional mitral valve MV. A catheter or catheter device 11 is positioned in the left atrium LA above the left ventricle LV. The catheter device is coupled to an implant 13. The implant can be positioned to repair the dysfunctional mitral valve MV.

While devices like this provide significant potential for potentially lifesaving repairs, there are still challenges. For example, one challenge is that, when treating a beating heart, the leaflets are in motion (both in shape and location) with the continuing operation of the heart. Thus, a repair device must navigate and operate relative to a moving target. Moreover, one or both leaflets of the dysfunctional valve may be deformed and/or damaged. As a result, the leaflets might not regularly move into a position and configuration in which they can be readily treated by the repair device. Moreover, the two or three target leaflets might have unusually large gaps therebetween, and they might be tethered into an unnaturally open position, such as by changes in the underlying structure (e.g., a dilated ventricle).

### SUMMARY

This summary provides exemplary features, and it is not intended to be limiting of the scope of the invention in any way. For example, any feature included in an example of this summary is not required by the claims, unless the claims explicitly recite the features. Also, the features, components, steps, concepts, etc. described in examples in this summary and elsewhere in this disclosure can be combined in a variety of ways. Various features and steps as described elsewhere in this disclosure may be included in the examples summarized here.

In some implementations, treatment or repair systems, devices, and related methods include helping to treat or repair native valves, including native two- and three-leaflet heart valves. The devices herein can be valve repair devices, implantable devices, valve treatment devices, implants, treatment devices, etc. While sometimes described as an implantable device or valve repair device for illustration purposes in examples herein, similar configurations can be used on other devices, e.g., treatment devices, removable devices, devices that are not necessarily implanted and can be removed after treatment, etc.

In some implementations, there is provided a device (e.g., treatment device, repair device, valve repair device, implantable device, implant, valve treatment device, etc.) that is configured to be positioned within a native heart valve to allow the native heart valve to form a more effective seal. The device can be part of a system (e.g., treatment system, repair system, valve repair system, valve treatment system, etc.).

In some implementations, the device includes an anchor portion. In some implementations, one or more anchors of the anchor portion are moveable between an open position and a closed position.

In some implementations, the device can be positioned within a heart valve, using a delivery system comprising catheters. In some implementations, the device can include leaflet anchors for attaching to valve leaflets, and one or more connectors forming decouplable or detachable connections between the anchors and the delivery system. In some implementations, the one or more connectors are severable connectors forming severable connections between the anchors and the delivery system.

In some implementations, a set of tethers is actuatable to move the anchors closer and/or into contact after the connectors have decoupled or detached leaving the anchors separated from the delivery system. In some implementations, the device can optionally include a spacer positioned to be compressed by opposing surfaces of the anchors when around the spacer.

In some implementations, a system can include a transvascular delivery system including a steering catheter system and/or an implant catheter system. The system can also include a device (e.g., treatment device, repair device, valve repair device, implantable device, implant, valve treatment device, etc.) that is introduced via the implant catheter system. The implant catheter system can optionally be guided by a steering catheter system.

In some implementations, the device includes a plurality of anchors (e.g., leaflet anchors, tissue anchors, clips, clasps, toggle anchors, staples, rivets, darts, etc.) that attach to tissue (e.g., one or more leaflets, an annulus, etc.) of a valve.

In some implementations, the device includes one or more connectors or severable-connectors (e.g., a first anchor severable-connector, a second anchor severable-connector, and/or a third anchor severable-connector) that form respective decouplable or severable connections (e.g., a first severable connection, a second severable connection, and/or a third severable connection) between the anchors and the delivery system or implant catheter system (e.g., a third anchor severable-connector can form a third anchor severable connection).

In some implementations, the device includes a system, apparatus, or mechanism (e.g., controlled tether system, tether system, tether mechanism, tether apparatus, coaptation system, actuation system, connection system, connection mechanism, connection apparatus, control system, etc.) that extendibly connects the first anchor to the delivery system or implant catheter system. In some implementations the system does so independently of the respective anchor connector for its anchor.

In some implementations, the system, apparatus, or mechanism is actuatable to move each anchor closer to and/or into contact with the other anchors (and/or with another portion of the device, e.g., a central body) after the anchor connectors of each anchor are decoupled. This provides for the implant catheter system to move separately away from an anchor that is attached to a leaflet, to then position one or more other anchors more easily at other leaflets or leaflet locations.

In some implementations, the device can include an optional spacer or center body (e.g., resilient spacer, filler, wedge, coaptation element, sheet, barrier, etc.). In some implementations, the spacer or center body is positioned such that it can be compressed by opposing surfaces of a plurality of anchors (e.g., leaflet anchors, tissue anchors, clips, clasps, rivets, etc.). In some implementations, the spacer or center body can form a pliant body having a functionally low pliant compressibility with respect to obstructed blood flow such that its compression causes a dimensional expansion that obstructs or inhibits blood regurgitation back through the valve.

In some implementations, the various systems, apparatuses, devices, etc. herein can be configured such that after decoupling an anchor, center body, other component from the delivery or implant catheter, then the delivery or implant catheter can be coupled or recoupled to the anchor, center body, or other component. This can be done using a tether or similar device that allows freedom of movement in the decoupled state, but can be used (e.g., tensioned, etc.) to bring the delivery/implant catheter back into contact with the anchor, center body, or other component. In some implementations, after recoupling, the delivery or implant catheter can be used to (1) detach the anchor, center body, or other component from a first location, and/or (2) reposition and/or attach the anchor, center body, or other component to a second location (different from the first location) (e.g., to a different tissue location).

In some implementations, a treatment/repair system to treat/repair a native valve of a subject (e.g., a living subject, a simulation, etc.) includes an implant catheter and device (e.g., treatment device, repair device, valve repair device, implantable device, implant, valve treatment device, etc.). The device can be coupled and/or be couplable to the implant catheter. In some implementations, the device includes one, all, or some of a first leaflet anchor, a first anchor severable-connector, a second leaflet anchor, a controlled tether system, and an anchor-lock.

In some implementations, the first leaflet anchor has a first leaflet-attachment mechanism. In some implementations, the first anchor connector forms a decouplable/detachable connection having a coupled state and a decoupled state (e.g., a connected state and a detached state, an unsevered state and a severed state, etc.) between the first leaflet anchor and the implant catheter. In some implementations, the first anchor connector is a severable connector that forms a severable connection having an unsevered state and a severed state between the first leaflet anchor and the implant catheter.

In some implementations, the second leaflet anchor has a second leaflet-attachment mechanism. In some implementations, the second leaflet anchor is connectable to the implant catheter independently of the first anchor connector.

In some implementations, the controlled tether system extendibly connects the first leaflet anchor to the implant catheter. In some implementations, the controlled tether system is actuatable to move the first leaflet anchor with respect to the second leaflet anchor with the first anchor connector in its decoupled/detached/severed state.

In some implementations, the anchor-lock has a locking mechanism that constrains relative movement of the respective first and second leaflet anchors when the locking mechanism is locked.

In some implementations, the first leaflet anchor, the second leaflet anchor and the first anchor connector are mounted on the implant catheter.

In some implementations, the controlled tether system includes a first controlled tether extendibly fastening the first leaflet anchor to an optional center body (e.g., a spacer, coaptation element, plug, gap filler, wedge, barrier, sheet, pad, etc.). The center body is separably attached to the implant catheter by a center-body connector or severable-connector.

In some implementations, the first controlled tether serially extends from a first controlled-tether first-end segment at a proximal end of the implant catheter, through the center body positioned at a distal end of, slidably received through a first anchor slidable restraint on the first leaflet anchor, back through the center body, to a first controlled-tether second-end segment at the proximal end of the implant catheter.

In some implementations, the second leaflet anchor is inseparably connected to the center body.

In some implementations, the controlled tether system includes a first controlled tether serially extending from a first controlled-tether first-end segment at a proximal end of the implant catheter, through a center-body slidable restraint on a center body separably attached to a distal end of the implant catheter by a center-body connector, to a first controlled-tether second-end segment affixed to the first leaflet anchor.

In some implementations, the controlled tether system includes a first controlled tether serially extending from a first controlled-tether first-end segment at a proximal end of the implant catheter, through a center-body slidable restraint on a center body separably attached to a distal end of the implant catheter by a center-body connector, through a first anchor slidable restraint on the first leaflet anchor, and back to a first controlled-tether second-end segment affixed to the center body.

In some implementations, the treatment/repair device further includes a second anchor connector (e.g., a severable connector) forming a decouplable/detachable connection having a coupled state (e.g., a connected state, an unsevered state, etc.) and a decoupled state (e.g., a detached state, a severed state, etc.) between the second leaflet anchor and the implant catheter. In some implementations, the controlled tether system extendibly connects the second leaflet anchor to the implant catheter, and the controlled tether system is actuatable to move the second leaflet anchor relative to the first leaflet anchor with the second anchor connector in its severed state. In some implementations, the second anchor connector is connected to, mounted on, or part of the implant catheter.

In some implementations, the controlled tether system includes a first controlled tether extendibly fastening the first leaflet anchor to an optional center body (e.g., spacer, coaptation element, plug, wedge, barrier, sheet, pad, etc.) separably attached to the implant catheter by a center-body severable-connector. In some implementations, the first controlled tether serially extending from a first first-end segment at a proximal end of the implant catheter, through the center body positioned at a distal end of the implant catheter, slidably received through a first anchor slidable restraint on the first leaflet anchor, back through the center body, to a first second-end segment at the proximal end of the implant catheter.

In some implementations, the controlled tether system includes a second controlled tether extendibly fastening the second leaflet anchor to the center body, the second controlled tether serially extending from a second first-end segment at the proximal end of the implant catheter, through the center body, slidably received through a second anchor slidable restraint on the second leaflet anchor, back through the center body, to a second second-end segment at the proximal end of the implant catheter.

In some implementations, the controlled tether system includes an anchor-tether section and an extension-tether. In some implementations, the anchor-tether section includes a first anchor-tether segment and a second anchor-tether segment. Each respective anchor-tether segment extends from an anchor-tether segment connection point to the respective leaflet anchor. In some implementations, the extension-tether section is connected to the anchor-tether section and extends proximally along the implant catheter to a proximal end of the implant catheter.

In some implementations, the treatment/repair device further includes a third leaflet anchor having a third leaflet-attachment mechanism.

In some implementations, the treatment/repair device further includes a third anchor connector (e.g., a severable connector, etc.) forming a decouplable/detachable connection having a coupled state (e.g., a connected state, unsevered state, etc.) and a decoupled state (e.g., detached state, severed state, etc.) between the third leaflet anchor and the implant catheter.

In some implementations, the controlled tether system extendibly connects the third leaflet anchor to the delivery catheter or implant catheter.

In some implementations, the controlled tether system is actuatable to move the third leaflet anchor relative to one or more of the first and second leaflet anchors with the third anchor connector in its decoupled/detached state.

In some implementations, the locking mechanism constrains relative movement of the third leaflet anchor with respect to the first and second leaflet anchors when moved together and the locking mechanism is locked.

In some implementations, the controlled tether system includes a first controlled tether extendibly fastening the first leaflet anchor to a center body separably attached to the implant catheter by a center-body connector (e.g., a severable-connector, etc.). In some implementations, the first controlled tether serially extends from a first controlled-tether first-end segment at a proximal end of the implant catheter, through the center body positioned at a distal end of the implant catheter, slidably received through a first anchor slidable restraint on the first leaflet anchor, back through the center body, to a first controlled-tether second-end segment at the proximal end of the implant catheter.

In some implementations, the controlled tether system includes a second controlled tether extendibly fastening the second leaflet anchor to the center body, the second controlled tether serially extending from a second first-end segment at the proximal end of the implant catheter, through the center body positioned at the distal end of the implant catheter, slidably received through a second anchor slidable restraint on the second leaflet anchor, back through the center body, to a second second-end segment at the proximal end of the implant catheter.

In some implementations, the controlled tether system optionally includes a third controlled tether extendibly fastening the third leaflet anchor to the center body. In some implementations, the third controlled tether serially extends from a third controlled-tether first-end segment at the proximal end of the implant catheter, through the center body positioned at the distal end of the implant catheter, slidably received through a third anchor slidable restraint on the third leaflet anchor, back through the center body, to a third controlled-tether second-end segment at the proximal end of the implant catheter.

In some implementations, the controlled tether system includes an anchor-tether section and an extension-tether section. In some implementations, the anchor-tether section includes a first anchor-tether segment, a second anchor-tether segment and a third anchor-tether segment. Each respective anchor-tether segment extends from an anchor-tether segment connection point to the respective leaflet anchors. In some implementations, the extension-tether section is connected to the anchor-tether section and extends proximally along the implant catheter to a proximal end of the implant catheter.

In some implementations, the first leaflet anchor is a distal leaflet anchor. In some implementations, the second leaflet anchor is a proximal leaflet anchor. In some implementations, the controlled tether system includes an implant-controlled tether fastening the distal leaflet anchor to a proximal end of the implant catheter. In some implementations, the proximal leaflet anchor is slidably attached to the implant-controlled tether. In some implementations, the end-body slidably receives the implant-controlled tether, to slide along the implant-controlled tether and move the distal leaflet anchor closer to (e.g., near, in contact with, etc.) the proximal leaflet anchor.

In some implementations, the end-body forms the locking mechanism. In some implementations, the locking mechanism includes a cable lock.

In some implementations, the proximal leaflet anchor is slidably attached to the implant-controlled tether by a proximal anchor-tether section forming an eyelet that is threaded onto the implant-controlled tether.

In some implementations, the cable lock allows the implant-controlled tether to be pulled through the end-body in only one direction.

In some implementations, the implant-controlled tether includes a distal anchor-tether section attached to the distal leaflet anchor. In some implementations, the distal anchor-tether section forms an eyelet. In some implementations, the implant-controlled tether includes an extension-tether section having a first-end segment, a second-end segment, and an intermediate segment intermediate the first-end segment and the second-end segment. In some implementations, the intermediate segment loops through the eyelet.

In some implementations, the implant-controlled tether is unitary from the proximal end of the implant catheter to the distal leaflet anchor.

In some implementations, each leaflet anchor is a clamp-type anchor.

In some implementations, each leaflet anchor is a T-shaped toggle anchor.

In some implementations, one or more of the treatment/repair systems herein include a center body or spacer (e.g., a resilient spacer, etc.). The first and second leaflet anchors form opposing surfaces. The spacer is positioned to be compressed by the opposing surfaces. The spacer forms a pliant body having a functionally low pliant compressibility with respect to obstructed blood flow.

In some implementations, a system (e.g., a tether system, coaptation system, connection system, controlled tether system, etc.) useable with the devices herein has a maximum level of force with which it can move together or connect the first leaflet anchor relative to the second leaflet anchor with the spacer positioned therebetween to receive the opposing surfaces. In some implementations, the system (e.g., tether system, connection system, etc.) can move the opposing surfaces of the anchors together with enough force to change the shape of the spacer or center body. In some implementations, when the system (e.g., a controlled tether system, etc.) is actuated at the maximum level of force, the spacer extrudes to extend beyond the opposing surfaces.

In some implementations, a device (e.g., treatment device, repair device, valve repair device, implantable device, implant, valve treatment device, etc.) is used in a system (e.g., treatment system, repair system, valve repair system, valve treatment system, etc.) to treat/repair a native valve of a subject (e.g., a living subject, a simulation, etc.). The native valve having a plurality of leaflets.

In some implementations, the system has a steering catheter system and/or one or more implant catheters. Each implant catheter of the one or more implant catheters has a respective catheter distal end for insertion into a steering catheter system proximal end, and a respective catheter proximal end for user manipulation of the device.

In some implementations, the device has one, some, or all of a first leaflet anchor, a second leaflet anchor, a first anchor severable-connector, a second anchor severable-connector, a system (e.g., a tether system, coaptation system, connection system, etc.), a center body, and an anchor lock. In some implementations, the first leaflet anchor and a second leaflet anchor each have a respective leaflet-attachment mechanism.

In some implementations, the first anchor connector is connected to the first leaflet anchor. In some implementations, the first anchor connector has a first catheter connector connecting to a first catheter distal end of a first implant catheter of the one or more implant catheters. In some implementations, the first anchor connector forms a decouplable or detachable connection having a coupled state (e.g., a connected state, unsevered state, etc.) and a decoupled state (e.g., a detached state, severed state, etc.) between the first leaflet anchor and the first implant catheter.

In some implementations, the second anchor connector is connected to the second leaflet anchor. In some implementations, the second anchor connector has a second catheter connector connected to a second catheter distal end of a second implant catheter of the one or more implant catheters. In some implementations, the second anchor connector forms a decouplable or detachable connection having a coupled state (e.g., a connected state, an unsevered state, etc.) and a decoupled state (e.g., a detached state, a severed state, etc.) between the second leaflet anchor and the second implant catheter.

In some implementations, the system (e.g., tether system, coaptation system, connection system, etc.) includes a first controlled tether connected to the first leaflet anchor and a second controlled tether connected to the second leaflet anchor. In some implementations, the first controlled tether extends from a first tether distal end at the first leaflet anchor to a first tether proximal end at a first catheter proximal end. In some implementations, the second controlled tether extends from a second tether distal end at the second leaflet anchor to a second tether proximal end at a second catheter proximal end. In some implementations, the system is actuatable to move the respective first and second leaflet anchors relative to each other, e.g., closer together and/or into contact with each other.

In some implementations, each of the respective first and second controlled tethers extend through the center body (e.g., spacer, coaptation element, etc.). In some implementations, the center body is slidable along the first and second controlled tethers such that the first leaflet anchor and/or the second leaflet anchor can be brought into contact with the center body.

In some implementations, the anchor-lock constrains the relative movement of the first and second leaflet anchors. In some implementations, the anchor-lock forms a cable lock allowing tethers to be pulled through the center body in only one direction.

In some implementations, the device includes a third anchor and a third anchor connector. In some implementations, the third leaflet anchor has a third leaflet-attachment mechanism. In some implementations, the third anchor connector is connected to the third leaflet anchor. In some implementations, the third anchor connector has a third catheter connector connecting to a third catheter distal end of a third implant catheter of the one or more implant catheters. In some implementations, the third anchor connector forms a decouplable/detachable connection having a coupled state (e.g., a connected state, an unsevered state, etc.) and a decoupled state (e.g., a detached state, a severed state, etc.) between the third leaflet anchor and the third implant catheter.

In some implementations, the system (e.g., a tether system, coaptation system, connection system, etc.) includes a third controlled tether connected to the third leaflet anchor. In some implementations, the third controlled tether extends from a third tether distal end at the third leaflet anchor to a third tether proximal end at a third catheter proximal end. In some implementations, the system is actuatable to move the first leaflet anchor, the second leaflet anchor, and/or the third leaflet anchor relative to each other.

In some implementations, an optional center body is slidable along the first, second and third controlled tethers to their respective first, second and third tether distal ends such that the first leaflet anchor, the second leaflet anchor, and the third leaflet anchor are brought closer together and/or in contact with each other and/or the center body.

In some implementations, the anchor-lock constrains the relative movement of the first, second and third leaflet anchors after they have been moved closer together.

In some implementations, the first controlled tether includes a first anchor-tether section attached to the first leaflet anchor at a first distal end of the first anchor-tether section. In some implementations, the first anchor-tether section forms a first eyelet at a first proximal end of the first anchor-tether section.

In some implementations, the first controlled tether includes a first extension-tether section having a first first-end segment, a first second-end segment, and a first intermediate segment. In some implementations, the first intermediate segment being intermediate the first first-end segment and the first second-end segment along the first extension-tether section. In some implementations, the first intermediate segment is looped through the first eyelet.

In some implementations, the second controlled tether includes a second anchor-tether section attached to the second leaflet anchor at a second distal end of the second anchor-tether section. In some implementations, the second anchor-tether section forming a second eyelet at a second proximal end of the second anchor-tether section. In some implementations, the second controlled tether includes a second extension-tether section having a second first-end segment, a second second-end segment, and a second intermediate segment.

In some implementations, the second intermediate segment being intermediate the second first-end segment and the second second-end segment along the second extension-tether section. In some implementations, the second intermediate segment being looped through the second eyelet.

In some implementations, the first catheter-connector has a quick-release mechanism to decouplably/detachably be first attached to the first implant catheter during an implantation, and then unattached during the implantation.

In some implementations, the device has an optional spacer or center body (e.g., a resilient spacer, a coaptation element, etc.) positioned and configured such that it can be compressed by opposing surfaces of the first and second leaflet anchors. In some implementations, the spacer forms a pliant body having a functionally low pliant compressibility with respect to obstructed blood flow. In some implementations, the system (e.g., a tether system, coaptation system, connection system, etc.) is actuatable at a level of force to move the first leaflet anchor relative to the second leaflet anchor with the spacer positioned to receive or contact the opposing surfaces.

In some implementations, the system (e.g., a tether system, coaptation system, connection system, etc.) defines a maximum level of force. In some implementations, with the system actuated at the maximum level of force, the spacer is compressed such that it extrudes to extend beyond the opposing surfaces.

In some implementations, a system (e.g., a treatment system, a repair system, a valve repair system, a valve treatment system, etc.) useable to treat or repair a native valve of a subject (e.g., a living subject, a simulation, etc.) includes an implant catheter and a device (e.g., treatment device, repair device, valve repair device, implantable device, implant, valve treatment device, etc.). The implant catheter has a catheter proximal end and a catheter distal end.

In some implementations, the device includes one, some, or all of a distal leaflet anchor having a distal leaflet-attachment mechanism, a distal anchor connector, a proximal leaflet anchor having a proximal leaflet-attachment mechanism and a proximal anchor slidable restraint, a proximal anchor connector, a controlled tether having a tether proximal end and a tether distal end, and an anchor-lock on the controlled tether.

In some implementations, the distal anchor connector forms a decouplable/detachable connection having a coupled state (e.g., a connected state, an unsevered state, etc.) and a decoupled state (e.g., a detached state, a severed state, etc.) between the distal leaflet anchor and the catheter distal end. In some implementations, the proximal anchor connector forms a decouplable or detachable connection having coupled state (e.g., a connected state, an unsevered state, etc.) and a decoupled state (e.g., a detached state, a severed state, etc.) between the proximal leaflet anchor and the catheter distal end.

In some implementations, the decouplable/detachable connection of the proximal anchor connector is independent from the decouplable/detachable connection of the distal anchor connector.

In some implementations, the tether distal end is connected to the distal leaflet anchor. In some implementations, the implant-controlled tether serially extends from the tether distal end, through the proximal anchor slidable restraint of the proximal leaflet anchor, and to the tether proximal end at the catheter proximal end.

In some implementations, the anchor-lock constrains proximal movement of the proximal leaflet anchor with respect to the implant-controlled tether.

In some implementations, the tether is formed from an extension-tether section and an anchor-tether section. In some implementations, the anchor-tether section is connected to the distal leaflet anchor and forms an eyelet. In some implementations, the extension-tether section has a first-end segment, a second-end segment, and an intermediate segment intermediate the first-end segment and the second-end segment. In some implementations, the intermediate segment loops through the eyelet.

In some implementations, the device includes an intermediate leaflet anchor and an intermediate anchor connector. In some implementations, the intermediate leaflet anchor has an intermediate leaflet-attachment mechanism and an intermediate anchor slidable restraint.

In some implementations, the intermediate anchor connector forms a decouplable/detachable connection having a coupled state (e.g., a connected state, an unsevered state, etc.) and a decoupled state (e.g., a detached state, a severed state, etc.) between the intermediate leaflet anchor and the catheter distal end. In some implementations, the decouplable/detachable connection of the intermediate anchor connector is independent from the decouplable/detachable connection of the distal anchor connector.

In some implementations, the tether extends through the intermediate anchor slidable restraint between the distal leaflet anchor and the proximal leaflet anchor.

In some implementations, the tether is unitary.

In some implementations, the device includes one or more spring spacers between a consecutive pair of anchors (e.g., tissue anchors, clips, clasps, toggle anchors, helical anchors, staples, rivets, darts, etc.).

In some implementations, the proximal anchor slidable restraint is a structurally integral part of the proximal leaflet anchor.

In some implementations, a device (e.g., treatment device, repair device, valve repair device, implantable device, implant, valve treatment device, etc.) usable to treat/repair a native valve of a subject (e.g., a living subject, a simulation, etc.) includes one or more of a first leaflet anchor, a second leaflet anchor, a spacer/coaptation element, and a system (e.g., a tether system, coaptation system, connection system, etc.).

In some implementations, the first leaflet anchor has a first leaflet-attachment mechanism. In some implementations, the second leaflet anchor has a second leaflet-attachment mechanism. In some implementations, the first leaflet-attachment mechanism is the same type of attachment mechanism as the second leaflet-attachment mechanism; however, in some implementations, they are different types of attachment mechanisms.

In some implementations, the respective first and second leaflet anchors have opposing surfaces. In some implementations, the spacer or coaptation element is positioned to be compressed by the opposing surfaces. In some implementations, the spacer forms a pliant body having a functionally low pliant compressibility with respect to obstructed blood flow.

In some implementations, the system (e.g., tether system, coaptation system, connection system, etc.) is actuatable at a level of force to move the first leaflet anchor relative to the second leaflet anchor, with the spacer positioned to receive the opposing surfaces.

In some implementations, the system defines a maximum level of force. In some implementations, with the system actuated at the maximum level of force, the spacer is compressed such that it extrudes to extend beyond the opposing surfaces.

In some implementations, the spacer is a unitary body.

In some implementations, the spacer is composed of a medical grade silicone.

In some implementations, the device includes an anchor-lock constraining relative movement of the first leaflet anchor and the second leaflet anchor when moved closer to and/or into contact with the spacer.

In some implementations, a system (e.g., a treatment system, a repair system, etc.) useable to treat/repair an organ or other body part of a subject (e.g., a living subject, a simulation, etc.) includes one or more of a delivery/implant catheter, a first anchor, a first anchor connector, a second anchor, and a controlled tether. In some implementations, the first anchor has a first attachment-body to attach to the organ and is mounted on the implant catheter.

In some implementations, the first anchor connector connects the first anchor to the implant catheter. In some implementations, the first anchor connector forms a decouplable connection having a coupled state (e.g., a connected state, an unsevered state, etc.) and a decoupled state (e.g., a detached state, a severed state, etc.) between the first anchor and the implant catheter.

In some implementations, the second anchor has a second attachment-body to attach to the organ. In some implementations, the second anchor is mounted on the implant catheter independently from the decouplable connection of the first anchor connector.

In some implementations, the controlled tether extendibly connects the first anchor to the implant catheter independent of the first anchor connector. In some implementations, the controlled tether is actuatable to move the first anchor relative to the second anchor.

In some implementations, the system includes an anchor-lock having a locking mechanism to constrain relative movement of the respective first and second anchors when they moved closer together and/or into contact (e.g., with each other and/or with a spacer).

In some implementations, a method for treating or repairing a native valve of a subject (e.g., a living subject, a simulation, etc.) includes providing a treatment/repair device on a distal end of a delivery catheter or implant catheter. The treatment/repair device includes one or more of a first leaflet anchor, a first anchor connector forming a first decouplable/detachable connection between the first leaflet anchor and the delivery/implant catheter, a second leaflet anchor connected to the delivery/implant catheter independently of the first anchor connector, and a system (e.g., a tether system, coaptation system, connection system, etc.) actuatable to move the first and second leaflet anchors relative to each other with the first decouplable/detachable connection being decoupled/detached.

In some implementations, the first leaflet anchor is positioned at the first leaflet. The first leaflet anchor is attached to the first leaflet. In some implementations, after the first leaflet anchor is attached to the first leaflet, the first anchor connector is decoupled, detached, severed, etc.

In some implementations, after the first anchor connector is decoupled, detached, severed, etc., the second leaflet anchor is positioned at the second leaflet. The second leaflet anchor is attached to the second leaflet. In some implementations, after the first and second leaflet anchors are attached to the respective first and second leaflets, the first and second leaflet anchors are brought together (e.g., closer together and/or into contact) using the system (e.g., tether system, coaptation system, connection system, etc.).

In some implementations, the treatment/repair device can be released from the implant catheter.

In some implementations, the treatment/repair device includes an anchor-lock having a locking mechanism that locks the relative positions of the first and second leaflet anchors with respect to one another. In some implementations, after the step of moving the first and second leaflet anchors, the relative positions of the first and second leaflet anchors are locked with respect to one another using the anchor-lock.

In some implementations, slack on the system (e.g., tether system, coaptation system, connection system, etc.) is controllably released before the step of attaching the second leaflet anchor to the second leaflet.

In some implementations, the plurality of leaflets includes a third leaflet. In some implementations, the device includes a second anchor connector forming a second decouplable/detachable connection between the second leaflet anchor and the implant catheter, and a third leaflet anchor connected to the implant catheter independently of the first and second anchor connectors.

In some implementations, the system (e.g., tether system, coaptation system, connection system, etc.) is actuatable to move the first, second and third leaflet anchors relative to each other after the first and second decouplable/detachable connections are decoupled/detached. In some implementations, after the second leaflet anchor is attached to the second leaflet, the second anchor connector is decoupled or detached. In some implementations, after the second anchor connector is decoupled or detached, the third leaflet anchor is positioned at the third leaflet. The third leaflet anchor is attached to the third leaflet. In some implementations, the first, second and third leaflet anchors are brought closer together and/or into contact with each other and/or with an optional center body/spacer between them.

In some implementations, the system or another system (e.g., tether system, coaptation system, connection system, etc.) can be configured such that after decoupling an anchor, center body, other component from the delivery or implant catheter, then the delivery or implant catheter can be coupled or recoupled to the anchor, center body, or other component. This can be done using a tether or similar device that allows freedom of movement in the decoupled state, but can be used (e.g., tensioned, etc.) to bring the delivery/implant catheter back into contact with the anchor, center body, or other component. In some implementations, after recoupling, the delivery or implant catheter can be used to (1) detach the anchor, center body, or other component from a first location (e.g., detach from tissue at a first tissue location), and/or (2) reposition and/or attach the anchor, center body, or other component to a second location (different from the first location) (e.g., to a different tissue location).

In some implementations, after the step of decoupling the first anchor connector, the method includes the step of moving the first leaflet anchor and the delivery/implant catheter together using the system (e.g., tether system, coaptation system, connection system, etc.). In some implementations, the method includes coupling (e.g., recoupling) the delivery/implant catheter to the first anchor. In some implementations, the method includes detaching the first leaflet anchor from the first location (e.g., from tissue at a first location) and attaching the first leaflet anchor at another location (e.g., a different tissue location).

Any of the above method(s) can be performed on a living subject (e.g., human or other animal) or on a simulation (e.g., a cadaver, cadaver heart, imaginary person, anthropomorphic ghost, simulator, such as a computer simulator, e.g., with the body parts, tissue, etc. being simulated). With a simulation, the body parts can optionally be referred to as “simulated” (e.g., simulated heart, simulated tissue, etc.) and can comprise, for example, computerized and/or physical representations.

Any of the above systems, assemblies, devices, apparatuses, components, etc. can be sterilized (e.g., with heat, radiation, ethylene oxide, hydrogen peroxide, etc.) to ensure they are safe for use with patients, and the methods herein can comprise (or additional methods comprise or consist of) sterilization of one or more systems, devices, apparatuses, components, etc. herein (e.g., with heat, radiation, ethylene oxide, hydrogen peroxide, etc.).

A further understanding of the nature and advantages of the implementations are set forth in the following description and claims, particularly when considered in conjunction with the accompanying drawings in which like parts bear like reference numerals. Other features and advantages will become apparent from the following detailed description and claims, particularly when considered with the accompanying drawings. The detailed description of implementations, as set out below to enable one to build and use the implementations, are not intended to limit the enumerated claims, but rather, they are intended to serve as examples of the claimed invention.

### BRIEF DESCRIPTION OF THE DRAWINGS

To further clarify various aspects of implementations of the present disclosure, a more particular description of the certain examples and implementations will be made by reference to various aspects of the appended drawings. These drawings depict only example implementations of the present disclosure and are therefore not to be considered limiting of the scope of the disclosure. Moreover, while the figures might be drawn to scale for some examples, the figures are not necessarily drawn to scale for all examples. Examples and other features and advantages of the present disclosure will be described and explained with additional specificity and through the use of the accompanying drawings in which:

FIG. 1 shows a cutaway view of a device positioned to repair a mitral valve.

FIG. 2 illustrates a cutaway view of the human heart in a diastolic phase;

FIG. 3 illustrates a cutaway view of the human heart in a systolic phase;

FIG. 4 illustrates a cutaway view of the human heart in a systolic phase showing valvular regurgitation;

FIG. 5 is the cutaway view of FIG. 4 annotated to illustrate mitral valve leaflets in the systolic phase;

FIG. 6 illustrates a healthy mitral valve with the leaflets closed as viewed from an atrial side of the mitral valve;

FIG. 7 illustrates a dysfunctional mitral valve with a visible gap between the leaflets as viewed from an atrial side of the mitral valve;

FIG. 8 illustrates a tricuspid valve viewed from an atrial side of the tricuspid valve;

FIG. 9 illustrates a cutaway view of a heart showing an implant catheter and treatment/repair device extended from within a steering catheter system and positioned within a mitral valve.

FIG. 10 illustrates details of the treatment/repair device illustrated in FIG. 9;

FIG. 10A illustrates the treatment/repair device of FIG. 10 with an implementation of a mechanism for opening and closing paddles of the device;

FIG. 10B illustrates an implementation of a mechanism for opening and closing paddles of the device of FIG. 10A;

FIGS. 10C and 10D illustrate an implementation of paddles that can be used in a device similar to the device of FIG. 10;

FIGS. 10E and 10F illustrate an implementation of a treatment/repair device that is similar to the treatment/repair device of FIG. 10;

FIG. 11 illustrates the implant catheter and treatment/repair device of FIG. 9, with a first leaflet anchor of the treatment/repair device located and oriented to clip on to a portion of a first leaflet.

FIG. 12 illustrates the implant catheter and treatment/repair device of FIG. 11, with the first leaflet anchor clipped on to the first leaflet and released from a central body of the treatment/repair device, and with a second leaflet anchor of the treatment/repair device located and oriented to clip on to a portion of a second leaflet.

FIG. 13 illustrates the implant catheter and treatment/repair device of FIG. 12, with the first and second leaflet anchors clipped on to the respective first and second leaflets and released from the central body, and with the central body positioned at a central, coaptation location between the leaflets.

FIG. 14 illustrates the treatment/repair device of FIG. 13, with the anchors connected to, and locked into position with respect to, a center body, and with the implant catheter disconnected from the treatment/repair device and removed from the subject's body.

FIG. 15 illustrates a view of an example 3-anchor implementation of the treatment/repair device of FIG. 9, positioned within a tricuspid valve.

FIG. 16 illustrates the treatment/repair device of FIG. 15, with a first leaflet anchor of the treatment/repair device located and oriented to clip on to a portion of a first leaflet.

FIG. 17 illustrates the treatment/repair device of FIG. 16, with the first leaflet anchor clipped on to the first leaflet and released from a central body of the treatment/repair device, and with a second leaflet anchor of the treatment/repair device located and oriented to clip on to a portion of a second leaflet.

FIG. 18 illustrates the treatment/repair device of FIG. 17, with the first and second leaflet anchors respectively clipped on to the first and second leaflets and released from the central body, and with a third leaflet anchor of the treatment/repair device located and oriented to clip on to a portion of a third leaflet.

FIG. 19 illustrates the treatment/repair device of FIG. 18, with the first, second and third leaflet anchors respectively clipped on to the first, second and third leaflets, and released from the central body, and with the central body positioned at a central, coaptation location between the leaflets.

FIG. 20 illustrates the treatment/repair device of FIG. 19, with the anchors moved into contact with the center body.

FIG. 21 illustrates an example expansion center body of the treatment/repair device of FIG. 20.

FIGS. 22A-22D illustrate a consecutively inserted, center-coupled implementation of a treatment/repair system.

FIG. 23 is a cutaway view of a center body of the consecutively inserted, center-coupled implementation of a treatment/repair system of FIG. 22, being connected to an implant catheter and tethered anchors.

FIG. 24 is a cutaway view of an example center body, including a spacer.

FIG. 25 is a cutaway view of an example center body.

FIG. 26 illustrates an example end-coupled implementation of a treatment/repair system.

FIG. 27 illustrates an example end-coupled implementation of a treatment/repair system.

FIGS. 27A and 27B illustrate an implementation of the treatment/repair system of FIG. 26 or 27 on a native mitral valve.

FIG. 28 illustrates an example end-coupled implementation of a treatment/repair system.

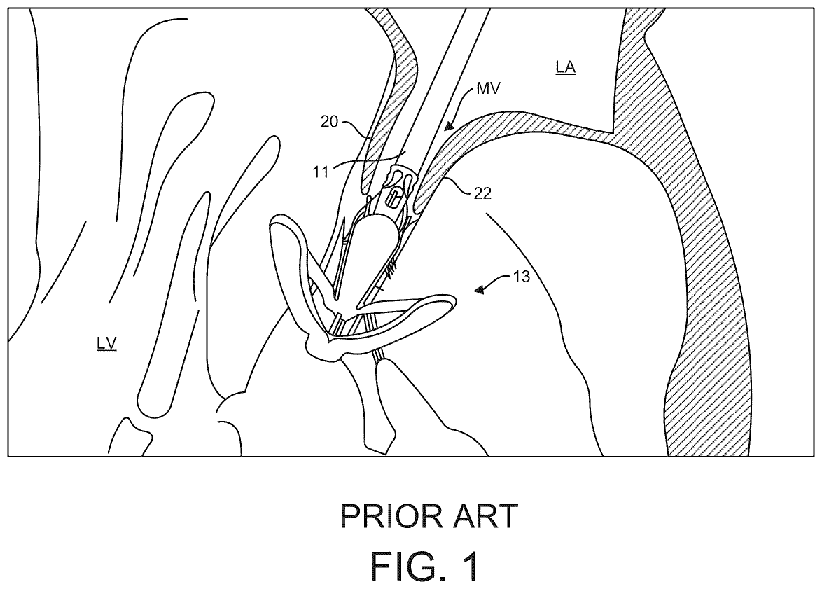

### DETAILED DESCRIPTION

The following description refers to the accompanying drawings, which illustrate example implementations of the present disclosure. Other implementations having different structures and operation do not depart from the scope of the present disclosure.

Some implementations of the present disclosure are directed to systems, devices, methods, etc. for treating or repairing a defective heart valve. For example, various implementations of devices, valve repair devices, implantable devices, implants, and systems (including systems for delivery thereof) are disclosed herein, and any combination of these options can be made unless specifically excluded. In other words, individual components of the disclosed devices and systems can be combined unless mutually exclusive or otherwise physically impossible.

The techniques, methods, operations, steps, etc. described or suggested herein or in the references incorporated herein, and any methods of using the systems, assemblies, apparatuses, devices, etc. herein, can be performed on a living subject (e.g., human, other animal, etc.) or on a simulation/simulated subject (e.g., a cadaver, cadaver heart, simulator, imaginary person, etc.). When performed on a simulation, the body parts, e.g., heart, tissue, valve, etc., can be assumed to be simulated or can optionally be referred to as “simulated” (e.g., simulated heart, simulated tissue, simulated valve, etc.) and can optionally comprise computerized and/or physical representations of body parts, tissue, etc. The term “simulation” covers use on a cadaver, computer simulator, imaginary person (e.g., if they are just demonstrating in the air on an imaginary heart), etc.

The examples summarized above and defined by the enumerated claims may be better understood by referring to the following detailed description, which should be read with the accompanying illustrative drawings, in which like parts bear like reference numerals. This detailed description disclosing examples, set out below to enable one to build and use the implementations, is not intended to limit the enumerated claims, but rather, it is intended to provide examples of them. Therefore, implementations having differing structures and methods, will not depart from the scope of the invention defined in the claims.

As described herein, when one or more components are described as being connected, joined, affixed, coupled, attached, or otherwise interconnected, such interconnection can be direct as between the components or can be indirect such as using one or more intermediary components. Also as described herein, reference to a “member,” “element,” “component,” or “portion” shall not be limited to a single structural member, component, or element but can include an assembly of components, members, or elements. Also as described herein, the terms “substantially” and “about” are defined as at least close to (and includes) a given value or state (preferably within 10 of and more preferably within 1 of).

Numerous variations of the example implementations described herein are within the scope of the invention, some of which are described for various implementations. To the extent they are viable, these variations are also envisioned for use with other implementations. Thus, a treatment or repair device herein may include any combination or sub-combination of the features and variations disclosed herein, and it may exclude some described features.

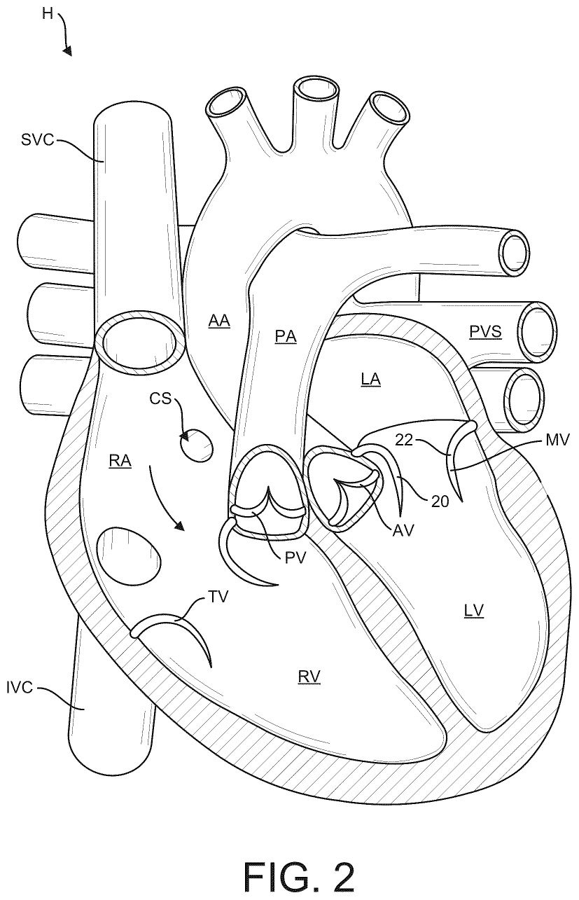

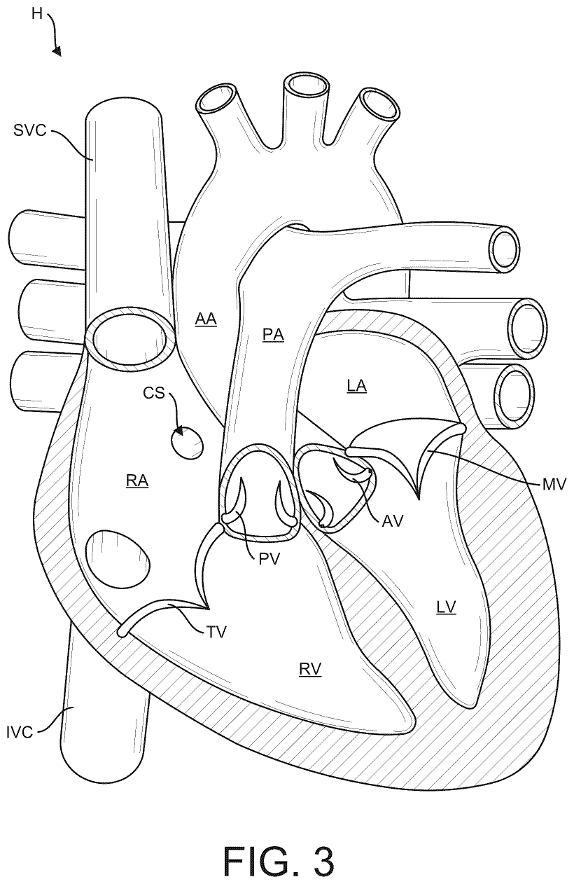

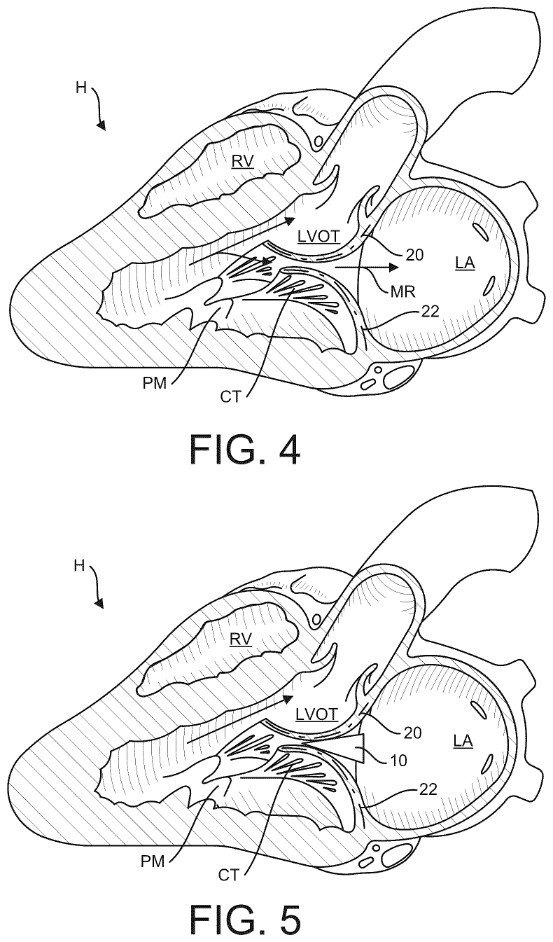

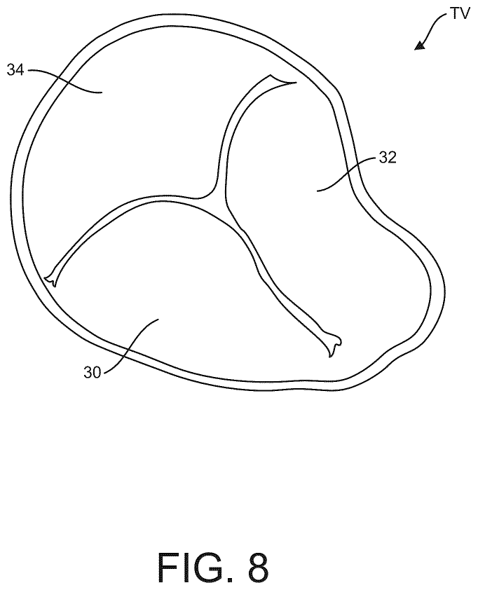

FIGS. 2-3 show cutaway views of a human heart H during distinct phases of a cardiac (heartbeat) cycle. A right ventricle RV and a left ventricle LV are separated from a right atrium RA and a left atrium LA, respectively, by a tricuspid valve TV and a mitral valve MV; i.e., the atrioventricular valves. Additionally, an aortic valve AV separates the left ventricle LV from an ascending aorta AA, and a pulmonary valve PV separates the right ventricle from a pulmonary artery PA. Each of these valves defines respective orifices, and has flexible leaflets (e.g., mitral valve anterior and posterior leaflets 20, 22 as also depicted in FIGS. 4-7, and tricuspid valve leaflets 30, 32, 34 as depicted in FIG. 8) extending inward across the respective orifices that come together or “coapt” in the flow stream to form the one-way, fluid-occluding surfaces. Organ repair systems, and more particularly native heart valve repair systems of the present application are frequently described and/or illustrated with respect to the mitral valve MV; and therefore, the anatomical structures of the mitral valve, the left atrium LA and left ventricle LV will be explained in greater detail. However, the devices described herein can also be used to repair other native valves, e.g., the devices can be used to repair a tricuspid valve TV, an aortic valve AV, a pulmonary valve PV, and/or an atrioventricular valve.

The left atrium LA receives oxygenated blood from the lungs via pulmonary veins PVS. At the beginning of the cardiac cycle, during a diastolic phase, or diastole (as depicted in FIG. 2), blood that was previously collected in the left atrium LA (during the end of a prior cardiac cycle) moves through the mitral valve MV and into the left ventricle LV by expansion of the left ventricle LV. At the same time, additional blood flows into the left atrium from the pulmonary veins. During a subsequent systolic phase, first there is an atrial systole in which the atria contract, and then there is a ventricular systole in which the ventricles contract.

In the atrial systole of the systolic phase, the left atrium contracts to eject additional blood from within the left atrium downstream into the left ventricle. In the ventricular systole of the systolic phase, as depicted in FIG. 3, the left ventricle LV contracts to force the blood through the aortic valve AV to the ascending aorta AA, where it circulates back through the rest of the body. During ventricular systole, the two leaflets of the mitral valve MV close to prevent the blood from regurgitating from the left ventricle LV back into the left atrium LA. At the same time, with the left atrium relaxing, blood is again collected in the left atrium from the pulmonary vein for the start of the next cardiac cycle.

The right atrium RA receives deoxygenated blood from the body via two veins, a posterior (inferior) vena cava IVC and an anterior (superior) vena cava SVC; and it receives deoxygenated blood from the coronary venous network via the coronary sinus CS. At the beginning of the cardiac cycle, during the diastolic phase, blood that was previously collected in the right atrium RA during the end of the prior cardiac cycle moves through the tricuspid valve TV and into the right ventricle RV by expansion of the right ventricle RV. At the same time, additional blood flows into the right atrium from the vena cava. In the atrial systole of the systolic phase, the right atrium contracts to eject additional blood from within the right atrium downstream into the right ventricle. In the ventricular systole of the systolic phase, the right ventricle RV contracts to force the blood through the pulmonary valve PV to the pulmonary artery PA, where it flows to the lungs. During ventricular systole, the three leaflets of the tricuspid valve TV close to prevent the blood from regurgitating from the right ventricle RV back into the right atrium RA. At the same time, blood is collected in the right atrium from the vena cava for the start of the next cardiac cycle.

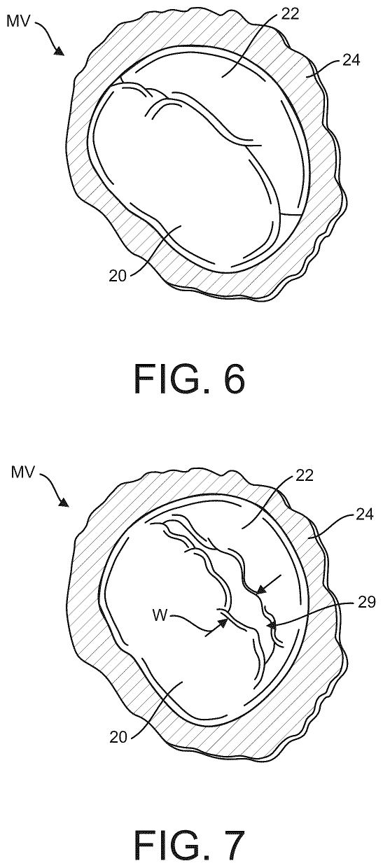

Referring now to FIGS. 2-8, the mitral valve MV includes two leaflets, an anterior leaflet 20 and a posterior leaflet 22. The mitral valve MV also includes an annulus 24 (see FIG. 6), which is a variably dense fibrous ring of tissues that encircles the anterior and posterior leaflets 20, 22. Referring to FIGS. 4-5, the mitral valve MV is anchored to a wall of the left ventricle LV by chordae tendineae CT. The chordae tendineae CT are a plurality of cord-like tendons that connect papillary muscles PM (i.e., the muscles located at the base of the chordae tendineae CT and within the walls of the left ventricle LV) to the anterior and posterior leaflets 20, 22 of the mitral valve MV. The papillary muscles PM serve to limit the movements of the anterior and posterior leaflets 20, 22 of the mitral valve MV and prevent the mitral valve MV from being reverted.

The mitral valve MV opens and closes in response to pressure differences between the left atrium LA and the left ventricle LV. Rather than opening or closing the mitral valve MV, the papillary muscles PM support and/or brace the anterior and posterior leaflets 20, 22 against the high pressure occurring in the left ventricle LV, that pressure being needed to circulate blood throughout the body. Together the papillary muscles PM and the chordae tendineae CT are known as parts of the subvalvular apparatus, which functions to keep the mitral valve MV from prolapsing or flailing into the left atrium LA when the mitral valve closes. As depicted in a Left Ventricular Outflow Tract (LVOT) view depicted in FIG. 4, the anatomy of the anterior and posterior leaflets 20, 22 is such that the inner sides of the leaflets coapt at their free end portions, and from there the anterior and posterior leaflets 20, 22 recede or spread apart from each other. The anterior and posterior leaflets 20, 22 spread apart in the atrial direction, until each leaflet meets with the mitral annulus 24.

Various disease processes can impair proper function of one or more of the native valves of the heart H. These disease processes include degenerative processes (e.g., Barlow's Disease, fibroelastic deficiency, etc.), inflammatory processes (e.g., Rheumatic Heart Disease), and infectious processes (e.g., endocarditis, etc.). In addition, damage to the left ventricle LV or the right ventricle RV from prior heart attacks (i.e., myocardial infarction secondary to coronary artery disease) or other heart diseases (e.g., cardiomyopathy, etc.) can distort a native valve's geometry, which can cause the native valve to dysfunction.

Dysfunctional native valves malfunction in diverse ways, including: (1) valve stenosis; and (2) valve regurgitation. Although both stenosis and regurgitation can affect any valve, stenosis is often found to affect the aortic and pulmonary valves, and regurgitation is often found to affect the atrioventricular valves (i.e., the mitral and tricuspid valves).

Both valve stenosis and valve regurgitation increase the workload of the heart H and may lead to serious conditions if left untreated, such as endocarditis, congestive heart failure, permanent heart damage, and cardiac arrest, possibly leading to an ultimately death. Because the left side of the heart (i.e., the left atrium LA, the left ventricle LV, the mitral valve MV, and the aortic valve AV) are primarily responsible for circulating the flow of blood throughout the body, there are substantially higher pressures on the left side heart, and a dysfunction of the mitral valve MV or the aortic valve AV is particularly problematic and often life threatening.

Valve stenosis can cause a native valve not to open properly and/or completely and thereby cause improper blood flow (e.g., obstructed blood flow, jetting, etc.). Often, valve stenosis results from buildup of calcified material on the leaflets of a valve, which causes the leaflets to thicken and impairs the ability of the valve to function properly.

Valve regurgitation generally occurs when the leaflets of the valve do not close completely, thereby causing blood to leak back into the prior (upstream) chamber (e.g., a mitral valve failure to close completely, causing blood to leak from the left ventricle to the left atrium). There are three main mechanisms by which a native valve becomes regurgitant (e.g., incompetent), which include Carpentier's type I, type II, and type III malfunctions.

A Carpentier type I malfunction involves the dilation of an annulus such that normally functioning leaflets are distracted from each other and fail to form a tight seal (e.g., the leaflets do not coapt properly). Included in a type I mechanism malfunction are perforations of the leaflets, as are present in endocarditis. A Carpentier type II malfunction involves prolapse of one or more leaflets of a native valve above a plane of coaptation. A Carpentier type III malfunction involves restriction of the motion of one or more leaflets of a native valve such that the leaflets are abnormally constrained below the plane of the annulus. Leaflet restriction can be caused by rheumatic disease or dilation of a ventricle.

Malfunctioning native heart valves may need to be replaced or repaired. Replacement can involve replacing the subject's native valve with a biological or mechanical substitute. Repair can involve the preservation and correction of the subject's native valve. In some implementations, the devices described by the present application are used to repair the function of a defective native valve. That is, the devices are configured to help close the leaflets of the native valve to inhibit blood from regurgitating. Some of the devices described in the present application are designed to easily grasp and secure the native leaflets. In some implementations, the native leaflets are grasped and/or secured around a coaptation element (e.g., a spacer, gap filler, wedge, plug, foam, sponge, etc.) that beneficially acts as a filler in the regurgitant orifice to help inhibit back flow or regurgitation, though this is not necessary for all implementations.

Many patients undergoing valve surgery, such as surgery to the mitral valve MV, suffer from a degenerative disease that causes a malfunction in a leaflet of a native valve (e.g., the mitral valve MV), which can result in prolapse, flail, and/or regurgitation. Referring to FIG. 6, when a healthy mitral valve MV is in a closed position, the anterior leaflet 20 and the posterior leaflet 22 coapt, which prevents blood from leaking from the left ventricle LV to the left atrium LA. Referring to FIGS. 4 and 7, mitral regurgitation MR can occur when the anterior leaflet 20 and/or the posterior leaflet 22 of the mitral valve MV is displaced such that the anterior and posterior leaflets 20, 22 do not properly coapt or close. This failure to coapt causes a gap 26 between the anterior leaflet 20 and the posterior leaflet 22, which can allow blood to flow backwards (e.g., into the left atrium LA from the left ventricle LV, as illustrated by the mitral regurgitation MR flow path depicted in FIG. 4).

Referring to FIG. 7, the gap 26 may have a width W between about 2.5 mm and about 17.5 mm, between about 5 mm and about 15 mm, between about 7.5 mm and about 12.5 mm, or about 10 mm. In some situations, the gap 26 may have a width W greater than 15 mm or even 17.5 mm. As set forth above, there are several different ways that a leaflet (e.g., anterior and posterior leaflets 20, 22 of mitral valve MV) may malfunction, which can thereby lead to valvular regurgitation.

In any of the above-mentioned situations, improvement of the valve function might be achievable using a treatment/repair device or implant capable of engaging the anterior leaflet 20 and the posterior leaflet 22 to close the gap 26 and prevent the regurgitation of blood through the mitral valve MV. As depicted in FIG. 5, an abstract representation of a device 10, or implant, is shown implanted between the anterior and posterior leaflets 20, 22 such that regurgitation does not occur during systole (compare FIG. 4 with FIG. 5).

In some implementations, the coaptation element (e.g., spacer, coaptation element, gap filler, plug, wedge, sheet, barrier, etc.) of the device 10 has a generally tapered or sloped shape (e.g., triangular shape, etc.) that naturally adapts to the native valve geometry and to its expanding leaflet nature (toward the annulus 24). In this application, the terms spacer, coaptation element, and gap filler are used interchangeably and refer to an element that fills a portion of the space between native valve leaflets and/or that is configured such that the native valve leaflets engage or “coapt” against (e.g., such that the native leaflets coapt against the coaptation element, spacer, etc. instead of only against one another).

The mitral valve MV and the tricuspid valve TV are more prone to deformation of leaflets and/or surrounding tissue, which, as described above, can prevent the mitral valve MV or tricuspid valve TV from functioning properly, allowing for the regurgitation or back flow of blood from the ventricle into the atrium (e.g., a deformed mitral valve MV may allow for regurgitation or back flow from the left ventricle LV to the left atrium LA, as depicted in FIG. 4). The regurgitation or back flow of blood from the ventricle to the atrium results in valvular insufficiency. Deformations in the structure or shape of the mitral valve MV or the tricuspid valve TV are often repairable. In addition, regurgitation can occur due to the chordae tendineae CT becoming dysfunctional (e.g., the chordae tendineae CT may stretch or rupture), which allows the anterior leaflet 20 and the posterior leaflet 22 to be reverted such that blood is regurgitated into the left atrium LA. The problems occurring due to dysfunctional chordae tendineae CT can be repaired by repairing the chordae tendineae CT or the structure of the mitral valve MV (e.g., by securing the anterior and posterior leaflets 20, 22 at the affected portion of the mitral valve).

With reference to FIGS. 7-8, the systems, devices, procedures, and concepts disclosed herein are described in detail for treating/repairing the structure of a native valve, often using a mitral valve as an illustrative example. On the left side of a heart the systems, devices, and procedures can be used between the anterior and posterior leaflets 20, 22 of the mitral valve MV to connect them and help prevent or inhibit regurgitation of blood from the left ventricle into the left atrium. The devices, procedures and concepts disclosed herein can also be used to treat/repair a single leaflet of a native valve.

Like the mitral valve, the other valves (e.g., aortic, pulmonic, and tricuspid valves) can be treated/repaired using the devices, procedures and concepts disclosed herein. For example, like the left side of the heart, on the right side of a heart such devices, procedures and concepts can be used between any two of the three leaflets on the tricuspid valve (e.g., the anterior leaflet 30, the septal leaflet 32 and the posterior leaflet 34) to connect them and prevent or inhibit regurgitation of blood from the right ventricle into the right atrium. Moreover, the devices, procedures and concepts disclosed herein can be used between all three tricuspid valve leaflets 30, 32, 34 to connect all three of them and prevent or inhibit regurgitation of blood from the right ventricle to the right atrium.

The devices, procedures, systems, and concepts disclosed herein can also be used to treat/repair a single leaflet of a valve. In sum, depending on the situation, the described devices, procedures, systems, and concepts might be used to treat or repair any native valve, as well as any component of a native valve.

In some implementations, the systems herein can be configured as organ repair systems for repairing an organ, e.g., the heart, or a valve of a heart. In some implementations, the systems can be configured to treat/repair another part of the body. In some implementations, the systems herein are valve treatment/repair systems for repairing or treating a valve. In some implementations, the systems herein include a treatment and/or repair device (e.g., a valve repair device, a valve repair implant, a valve treatment device, a treatment device, an implant, a device, etc.).

In some implementations, the treatment/repair systems herein comprise a delivery system having a distal end that is inserted into a subject (e.g., a living patient, a simulation, etc.), and a proximal end that remails outside of the subject. The delivery system can include an implant catheter system having catheter components (e.g., delivery and/or implant catheters) configured and sized to receive, deliver, and implant, or otherwise deploy, the treatment/repair device within a subject.

The treatment/repair devices herein can be used by medical personnel in repairing or treating the subject's native valve. In some implementations, this is done by repairing and/or supporting one or more leaflets of the subject's valve.

In some implementations, the device can be delivered to the valve by the delivery system through the subject's vascular system. The delivery and/or implant catheter(s) can have a distal end for insertion into a proximal end of a steering catheter system, and a proximal end for manipulation by a user of the delivery system.

In some implementations, the system is provided with an array of controllable parts that are for various actuation activities, some of which are described herein and others which are known in the art. In some implementations, the system is configured such that a user (e.g., medical personnel) can operate various controls (e.g., one or more of knobs, motors, buttons, switches, sliders, gears, motors, screws, cams, gimbles, etc.) to control the system. In some implementations, the system can comprise mechanisms known to be useable in other transvascular devices, though the actuation activities and related controllable parts themselves may be unique.

In some implementations, the system is provided with a wide array of related parts and methods, such as may be described herein, either directly or indirectly.

Additional details and background information on systems, devices, and methods for transvascular heart valve treatment/repair (with associated mechanisms, components, operations, steps, etc.) that can be used with the systems, devices, methods, etc. herein can be found in U.S. Pat. No. 8,449,599 and U.S. Patent Application Publication Nos. 2014/0222136, 2014/0067052, 2016/0331523, and PCT patent application publication Nos. WO2020/076898, each of which is incorporated herein by reference in its entirety for all purposes. In some implementations, leaflet anchors described in any of these incorporated references can optionally be used as leaflet anchors in the implementations described herein. Also, coaptation elements, spacers, etc. described in any of these incorporated references can optionally be used as a center body in any of the implementations described herein.

An example transvascular technique that can be used with the systems, devices, methods, etc. herein for treating/repairing a native valve can comprise advancing a catheter or catheter device into an atrium (e.g., inserting the catheter device into the right femoral vein, up the inferior vena cava and into the right atrium). In some implementations, the tricuspid valve is treated from the right atrium. In transeptal transvascular techniques, the septum can be punctured from the right atrium, and the catheter device passed into the left atrium. In some instances, the catheter device can be coupled to a device (e.g., treatment device, repair device, implant, etc.). The device can be positioned to repair a dysfunctional native valve.

The delivery systems herein can comprise one or more components forming a steering catheter system and/or an implant catheter system. In some implementations, the delivery systems can have one or more components forming a steering catheter system and an implant catheter system.

In some implementations, the implant catheter system includes one or more implant catheters capable of placing and implanting, or otherwise deploying, a treatment or repair device. Optionally, the one or more implant catheters can be steerable catheters capable of navigating a subject's vascular system, thereby also forming all or part of the steering catheter system rather than being a separate system. The delivery system can further include additional devices for transvascular procedures, such as sheaths, control guidewire, external catheter control systems and controls (e.g., one or more of knobs, motors, buttons, switches, sliders, gears, motors, screws, cams, gimbles, etc.), and the like.

In some implementations, a steering catheter system includes one or more steering catheters capable of navigating a subject's vascular system.

In some implementations, e.g., ones having a separate steering catheter system, the one or more steering catheters can be either individually or collectively configured to guide the one or more implant catheters through the subject's vascular system. In some implementations, this can involve positioning a distal end of the steering catheter system upstream of a defective valve (relative to a blood flow direction) and pointing the distal end of the implant catheter system downstream (in the blood flow direction) through the valve.

In some implementations, the implant catheter system starts off inserted within the steering catheter system prior to its insertion into the subject's body or is inserted into a proximal end of the steering catheter system after the steering catheter system's distal end is positioned upstream of the valve. With the steering catheter system in place and the implant catheter inserted, the implant catheter system is extended downstream from within steering catheter system, toward the valve.

The component parts of the treatment/repair device can be configured to traverse the subject's vascular system through the delivery system in an operational configuration. In some implementations, the treatment/repair device can be structurally configured with a first, small-form-factor (e.g., small diameter) delivery configuration structurally laid out for traversing the delivery system through the subject's vascular system, and a second, operational configuration structurally appropriate for valve treatment or repair operations. In some such implementations, the implant catheter includes one or more actuators functional to actuate the treatment/repair device from the delivery configuration to the operational configuration while the device is located at its installation location within the subject's heart.

In some implementations, the component parts of the device can be configured for simultaneously traversing the subject's vascular system through the delivery system as a single implantable unit.

In some implementations, the component parts of the device can be configured to transvascularly traverse the delivery system through the subject's vascular system on a piece-by-piece basis. In some implementations, the pieces are assembled into an implantable device within the body (e.g., within the heart during an implantation procedure).

In some implementations herein, a treatment/repair system includes and uses a plurality of anchors (e.g., leaflet anchors, tissue anchors, clips, clasps, toggle anchors, staples, rivets, darts, etc.) that attach to tissue (e.g., tissue of an organ). In some implementations, the system is in the form of and/or comprises a treatment or repair device that includes at least one of or a plurality of independently actuatable leaflet anchors (e.g., clips, clasps, clamps, toggle anchors, staples, rivets, darts, knots, etc.) are also possible.

In some implementations, a leaflet anchor is an attachment device structured (once it is in an operational configuration) with a leaflet-attachment mechanism to affix itself to a leaflet, in any one or more of a variety of ways. This attachment mechanism can be, for example, in the form of an attachment-body (e.g., a bar). In some implementations, there will be a plurality of attachment-bodies (a first attachment-body, a second attachment-body, a third attachment-body, etc.), each of which can have any of a variety of cross sections (e.g., a relatively flat, paddle-type cross), and longitudinally extending in any of a wide variety of shapes (e.g., a straight line or a circle) to form compressive structures (e.g., paddles or rings).

In some implementations, the attachment-bodies can be configured affix to the leaflet, such as by pressing against the leaflet and thereby constraining the movement of the leaflet with respect to the anchor. For example, in some implementations of a clamp-type anchor, two attachment-bodies in the form of paddles can be configured such that they are attached at one end with a hinge to form a jaw that can bite down and clamp or clasp on to a leaflet and thereby affix to the leaflet. In some implementations, this clasp-type anchor attachment apparatus (or attachment mechanism) may be provided with two control apparatuses (or control mechanisms), one that actuates the attachment apparatus between an open configuration and an attached configuration (optionally using a spring to load the jaw towards an open or closed position), and one that locks the attachment apparatus onto a leaflet. In some implementations, the control apparatuses can be controlled using control wires, such as sutures, wires, lines, tethers, etc.

Likewise, any leaflet anchor herein can be provided with various other forms of tissue-attachment mechanisms such as friction devices, adhesion devices, tissue piercing mechanisms and/or various forms of grapnel. The anchor(s) can take and/or comprise a variety of forms, e.g., clasps, clamps, clips, darts, staples, hooks, bars, rods, screws, knots, toggle anchors, etc.

In some implementations, each leaflet anchor can comprise or be in the form of a toggle anchor, having a bar with a centrally attached respective anchor tether (e.g., a tether or tether section associated with a specific anchor). The bar can be made to longitudinally pierce, penetrate, and pass through a wall of a leaflet, pulling the anchor tether with it like a needle pulling a thread. To do this maneuver, the bar can be oriented parallel to the anchor tether, and then an end of the bar is pushed through a wall of a leaflet using a sharp point or edge (the point either being on an end of the bar or on a separate penetrator that contains or is attached to the toggle anchor). In some implementations, a structural support member can be used to support leaflet structures during penetration.

In some implementations, after passing through the wall, the anchor tether is pulled and the bar thereby toggles, (rotates) to laterally press against the wall of tissue with a distal (anchor) end of the anchor tether pulled back against, and extending back through, the penetrated wall of tissue in a T-shaped configuration (where the bar forms the top of an upper-case T). This T-shaped toggle anchor (also known as a T-element anchor) prevents the bar from passing back through the hole in the tissue (leaflet) when pulled by the anchor tether. The anchor can use variations for the structure of a toggle anchor bar, such as a bar made of an elongated accordion-folded fabric with the anchor tether extending through multiple leaves of the accordion that are then squeezed into an accordion-folded structure to forming the bar.

In some implementations, the device also includes one or more connectors, such as decouplable, detachable, or severable connectors. At least one of these one or more connectors is an anchor connector or anchor severable connector (e.g., a first anchor connector, a first anchor severable connector, a second anchor connector, a second anchor severable connector, etc.) in the form of a fastener or connection that fastens, attaches, or couples the structure of an associated anchor of the one or more anchors, either directly or indirectly, to the structure of a catheter of the delivery catheter system/implant catheter system.

In some implementations, each anchor connector structurally fastens, couples, attaches, etc. the anchor and catheter together rigidly. Each such anchor connector provides for the user (via the delivery or implant catheter system) to first position the associated anchor for affixing it to a valve portion like a leaflet, and then, after the anchor is affixed to that leaflet, with user actuation, to decouple, detach, or sever the connection provided by the anchor connector, and thereby unfasten and release the anchor from the implant catheter. The decoupling/detaching/severing of the anchor connector thereby removes that connector's constraint on the anchor from becoming separated from the implant catheter. The implant catheter can then move separately away from the anchor, to, for example, position one or more other anchors at other leaflets or leaflet locations.

In some implementations, a connector (e.g., a severable connector, an anchor connector, a catheter connector, etc.) can be a structure forming a connection that, in a first, unsevered state, joins and fastens the structures of two distinct components (e.g., an anchor and a delivery catheter) to the extent that they are constrained (typically with substantial rigidity) so as to move in sync with one another, in one or more degrees of freedom (and typically in six degrees of freedom). In some implementations, a severable connector can be controllably decouplable/detachable to a second, decoupled state (e.g., detached state, severed state, etc.) that breaks (ends) the constraint on the in-sync movement in at least one of the constrained degrees of freedom (and typically in six relative degrees of freedom) from one another. Put another way, the connector or severable connector forms a decouplable/detachable/severable connection between a first component and a second component. In some implementations, the decouplable/detachable/severable connection includes a coupled, connected, or unsevered state in which the connector constrains the first component and the second component to move in sync with one another in a set of one or more constrained degrees of freedom. In some implementations, the decouplable/detachable/severable connection can also include a decoupled, detached, or severed state in which the first component and the second component are not constrained by the connector to relatively move in sync with one another, e.g., in at least one constrained degree of freedom of the set of one or more constrained degrees of freedom.

In some implementations, two objects moving in sync with regard to one or more degrees of freedom encompasses a state in which the two objects move (e.g., translate and/or rotate) together in the one or more degrees of freedom, at the same time, to retain their positions relative to one another, such as would occur with a rigid connection between the two objects.

The transition from the coupled to decoupled states (e.g., from connected to detached states, from unsevered to severed states, etc.) can controllably occur via a variety of physical connection structures, such as being held by a retractable mechanical stop, being held by a clamp or clasp, being held by an integral connector that can be cut, etc. In some implementations, the connector structure can be formed by (e.g., include) integral portions of one or both of the two distinct components, and/or by wholly separate structural elements that attach to one or both of the two distinct components.

In some implementations, six degrees of freedom of the connector might be three translational degrees of freedom (e.g., respectively translating along a given set of x, y and z axes that are normal to one another, one of which might be parallel to the direction in which the end of the implant catheter is facing), and three rotational degrees of freedom (e.g., respectively rotating around the given set of x, y, and z axes). A rigid connection between two distinct components can be inflexible to the degree necessary for medical personnel to functionally control the position and orientation of one component (e.g., an anchor) using nothing but movement of the other component (e.g., a distal tip of an implant catheter). A certain degree of flexibility is inherent in any material (particularly in transvascular devices), but there is also a level of rigidity that can provide functionality for the device when used in this type of procedure.

In some implementations, at least some of the anchors (e.g., one or more clips, clasps, clamps, toggle anchors, staples, rivets, darts, knots, etc.) that are connected to the implant catheter system (and/or to other anchors) by connectors, are each further connected to a controlled tether system having one or more controlled tethers (e.g., sutures or other types of cords). In some implementations, each controlled tether connects its respective anchor to the implant catheter system such that control is maintained on the anchor via a controllable connection between the anchor and the implant catheter system after the connector decouples, detaches, releases, severs, etc. to break the connector's constraint between the relative movement of the implant catheter and the anchor.

In some implementations, either the controlled tether controllably connects the anchor to the implant catheter system separately from the implant catheter system's connection to other anchors, or the controlled tether connects the anchor to the implant catheter system via one or more other anchors of the plurality of anchors. In the case wherein the controlled tether separably connects the anchor to the implant catheter system, the controlled tether either may be affixed to the implant catheter system only at a distal catheter section that is within the subject's body or may extend back through the implant catheter system to a proximal end of the implant catheter system, so that it might be directly accessed by medical personnel.

In some implementations, a tether can be a structure physically joining two or more distinct components (e.g., joining an anchor and an implant catheter system, or an anchor and another anchor, or other components, etc.). In some implementations, a tether may join components such that they are flexibly unconstrained with regard to moving separately from one another in one or more (and typically six) degrees of freedom, but are constrained not to separate from one another (in one or more degrees of freedom) by farther than a defined tether limitation (e.g., an amount of tether slack, e.g., an available length of tether to extend). This unconstrained movement within a defined tether limitation can be large enough to allow for the independent movement of the components at a distance adequate to facilitate separate placement of the components in functionally independent locations (e.g., placement wherein a first leaflet anchor is attached to a first portion of a first leaflet while an implant catheter is positioned with a second leaflet anchor at a first portion of a second leaflet at a different location). In some implementations of a tether joining more than two distinct components, in combination the components can be constrained not to separate from one another (in one or more degrees of freedom) by farther than a defined tether limitation.

As used herein, this tether flexibility should be understood as being flexible to the degree necessary for medical personnel to functionally control the position and orientation of one component (e.g., a distal tip of an implant catheter) free of functionally significant impeding forces delivered through the tether from a contrary movement or position of another tethered component (e.g., a tethered anchor), so long as that tethered component is within the freedom of movement provided by the extent of the tether limitation. In simplified terms, the tether is flexible enough to allow anchors to be attached to leaflets at a greater distance from one another than anchors directly connected to one another. As such, mechanical extension devices other than a simple cord (e.g., a series of flexible rods and/or joints) may be within the scope herein, so long as they provide usable tether flexibility.

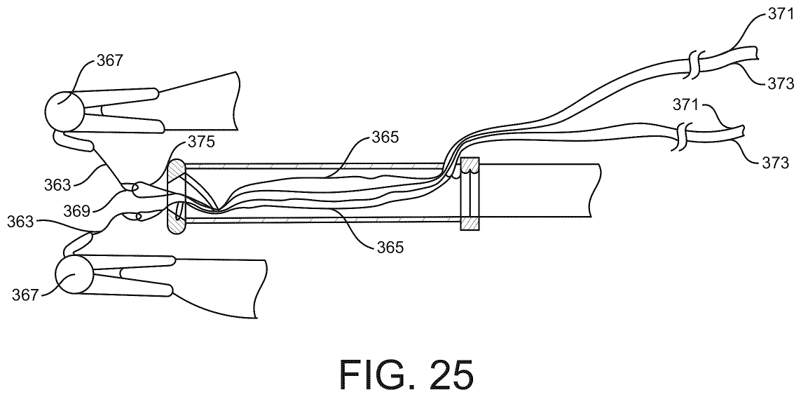

As used herein, a tether may be understood to include multiple individual (separate) sections of tether that are connected, such as by having one tether section loop through an eyelet formed by another tether section to form the tether as a whole. Also, as used herein, limited portions of tethers, and of their tether sections, will be referred to as segments of the respective tethers and/or tether sections. For example, a tether or tether section may be said to have a first-end segment, a second-end segment, and an intermediate or center segment (see, e.g., FIG. 25 and the related disclosure).

A controlled tether is a tether controllably configured such that the tether may be controllably actuated to move one or more connected components relative to other components (e.g., to coapt leaflet anchors, and thereby their respective leaflets, after the leaflet anchors are attached to their respective leaflets). A controlled tether can be formed by (e.g., include) integral portions of one or both joined components, and/or by wholly separate structural elements (such as sutures) that attach or pass through one or both of the two joined components.

In some implementations a controlled tether (or section thereof) may be a looped tether. In some implementations, a looped tether (or looped tether section) can connect from a proximal end, which is actuatable and removable from or with the implant catheter system (e.g., an end of which could remain outside the subject during initial insertion), to a distal end forming a loop (e.g., a bight) that connects to a primary connection point associated with some portion of a treatment or repair device. As a result, the looped tether can be used to controllably actuate or connect (e.g., hold together) that portion of the device while needed for implantation, and then the (looped) tether can be removed without physically cutting or severing device from the looped tether.

In some implementations, a looped tether is configured for this removable operation by serially extending (end-to-end) from a segment at a first end of the tether (e.g., a controlled-tether first-end segment or an extension-tether first-end segment), which is at a proximal end of the implant catheter system (protruding from a proximal, user end of the implant catheter), down along (through or aside) the implant catheter system to its distal (implantation) end, optionally through and out of a center body positioned at a distal end of the implant catheter system, to the tether's primary connection point (e.g., a respective anchor) of the device, slidably received by and passing through (looped around) that primary connection point, back into and through the center body (optionally), back along (through or aside) the implant catheter system to its proximal end, to a segment at a second end of the tether (e.g., a controlled-tether second-end segment or an extension-tether second-end segment) at the proximal end of the implant catheter system (protruding from the proximal, user end of the implant catheter along with the first-end segment of the looped tether). A looped tether of this type can be removed from a body while leaving an implanted implant by pulling on the first-end segment of the tether while releasing the second-end segment to be pulled around the path of the tether loop by the first-end segment. Other forms of looped tethers or other releasable tethers, which can be used to controllably actuate or connect (e.g., hold together) the device or components thereof, and then removed without physically cutting or severing the device, are within the scope of this disclosure as well.

In some implementations, the treatment or repair device includes a system (e.g., controlled tether system, tether system, adjustment system, coaptation system, actuation system, connection system, control system, etc.). In some implementations, the system can include a center body configured as a coaptation element and/or a spacer. The center body can be configured to be actuatable to guide (e.g., move, adjust, etc.) and maintain the position of the anchors, and thereby the leaflets. More particularly, with anchor connectors being in a decoupled or detached state and the center body positioned and located between the anchors and facing opposing surfaces of anchors, the center body operates to pull and guide leaflets, using a level of force (e.g., connection force, adjustment force, etc.) up to a maximum level of force (e.g., a maximum allowable level such as a safety limit), to a single location where they are closely positioned, held, and maintained to improve their operation as an operable valve closure element.

In some implementations, as a spacer, the center body is configured with a gap-filling shape, structure and composition that obstructs gaps between leaflets to reduce regurgitation. In some implementations, the center body structure is shaped and positioned with respect to the leaflets such that, upon coaptation, opposing surfaces of the anchors, and their respective leaflets, close around the center body during appropriate portions of the cardiac cycle. In some implementations, the center body/spacer can be configured to seal against opposing surfaces of two or three anchors, and thereby against two or three native-valve leaflets, depending in part upon the type of valve for which it is adapted. The center body/spacer can be shaped in any of a variety of shapes, as might be appropriate for the intended use. For example, the variety of shapes can include cylindrical capsule shapes, such as an elongated cylindrical shape characterized by a longitudinal cross-section that is: round, oval, ovoid, crescent shaped, or even rectangular. In some implementations, the center body can have an upstream (e.g., atrial, or upper) portion positioned in or adjacent to an atrium, a downstream (e.g., ventricular, or lower) portion positioned in or adjacent to the ventricle, and a side surface that extends between native leaflets. In some implementations, the spacer can, for example, be structured as a tubular-shaped braided wire composed of a nitinol mesh.

In some implementations, the spacer may be a compliant spacer with a low compressibility that, when mechanically compressed, compressively expands in some dimensions to be wider and/or longer (or expanding in one or more other dimensions) such that it both conforms to adjacent opposing surfaces and fills up adjacent spaces, and possibly extrudes therebeyond.

In some implementations, anchors are simultaneously inserted into the subject. In some implementations, at least some of the anchors are individually pulled to a central location by a system (e.g., controlled tether system, tether system, coaptation system, actuation system, connection system, control system, etc.) having distinct and separately extending controlled tethers. This can be done with two- and three-leaflet valves and can incorporate procedures for treating/repairing three-leaflet valves using only two leaflet anchors.

With reference to FIGS. 2-14, in some implementations (e.g., a simultaneously inserted, center-coupled implementation), of a treatment or repair system, a treatment or repair device 101 for use by medical personnel in treating or repairing a subject's native valve, is delivered through the vascular system to the valve by a delivery system for valve treatment/repair. In some implementations, the delivery system is provided with a steering catheter system including a steering catheter 103, which can operate similar to other steering catheter systems.

In some implementations, the delivery involves positioning a distal end of the steering catheter 103 upstream of the defective native valve (e.g., mitral valve MV, etc.) (relative to a blood flow direction 105) and pointing the distal end of the steering catheter downstream (in the blood flow direction) through the native valve.

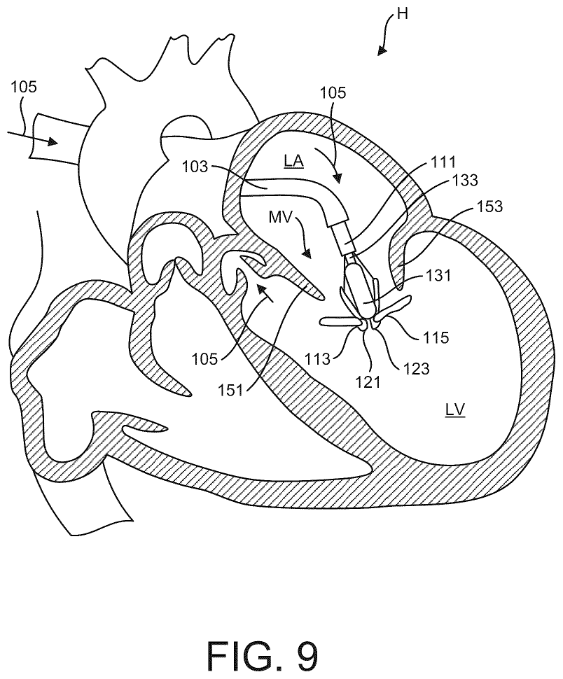

In some implementations, the delivery system is provided with a delivery catheter system or implant catheter system including a delivery catheter or implant catheter 111. In some implementations, the implant catheter starts off within a steering catheter system prior to its insertion into the subject's body. In some implementations, the implant catheter is inserted into a steering catheter system proximal end (which extends outside the subject's body for access by a user) after the steering catheter system is positioned upstream of the native valve. In some implementations, with the steering catheter system in place and the implant catheter inserted, a distal, implantation end of the implant catheter is extended from within steering catheter system downstream toward the native valve (e.g., as depicted in FIG. 9). In some implementations, the implant catheter can be steerable. In some implementations, the implant catheter may be used without a separate steerable catheter.

At its distal end, the delivery or implant catheter 111 mounts and carries some or all portions of a device 101 (e.g., treatment device, repair device, valve treatment device, valve repair device, implantable device, implant, etc.). In some implementations, device 101 comprise two leaflet anchors, e.g., a first leaflet anchor 113 and a second leaflet anchor 115. In some implementations, each anchor has a respective leaflet-attachment mechanism (e.g., a first leaflet-attachment mechanism, and a second leaflet-attachment mechanism) in the form of an actuatable clip with independent actuators and actuation controls such that each anchor can be separately and independently affixed to respective leaflet locations.

In some implementations, clip anchors can be configured such that each includes two paddles, an outer paddle 117 and an inner paddle 119. In some implementations, the two paddles are connected at one end with a hinge 120. In some implementations, these clip anchors are configured to affix to a leaflet by clipping onto a leaflet, e.g., by actuating the hinge to bite down onto, and thereby grasp, an edge of the leaflet requiring constraint for improved valve operation.

In some implementations, the device 101 also includes an anchor connector (e.g., a decouplable connector, a detachable connector, a severable connector, etc.) for each respective anchor, e.g., a first anchor connector 121 for the first leaflet anchor 113 and a second anchor connector 123 for the second leaflet anchor 115. In some implementations, the first anchor connector forms a first anchor decouplable connections (e.g., detachable connection, severable connection, releasable connection, etc.), and a second anchor connector forms a second anchor decouplable connection (e.g., detachable connections, severable connection, releasable connection, etc.). In some implementations, the device is further provided with an optional center body 131 (e.g., a spacer, a coaptation element, a plug, a gap filler, etc.) and a center-body connector 133. In some implementations, the center-body connector 133 is a structural fastener that forms a center-body decouplable connection (e.g., detachable connections, severable connection, releasable connection, etc.) that fastens the center body 131 to the delivery/implant catheter 111 when in a connected state or unsevered state (e.g., it restricts and/or constrains their relative movement to be in-sync).

In some implementations, the first and second connectors 121, 123 respectively form a first anchor decouplable connection and a second anchor decouplable connection that fasten their respective first and second leaflet anchors 113, 115 to the center body 131 when in a connected state or unsevered state, and thereby indirectly fasten the first and second anchor connectors to the delivery/implant catheter 111 via the center body. In some implementations, these first and second anchor decouplable connections are distinct and independent of one another in that the decoupling or detaching of one of the first anchor and second anchor connectors does not decouple or detach the connection made by the other connector (e.g., it does not release the constraint to move in-sync). Various connector technologies and/or mechanisms, such as those for decoupling or detaching implants from implant catheters (e.g., clasps, clamps, releases, ties, fasteners, loops, etc.), may be appropriate for use as the connectors or as part of the connectors.

In their connected states, the connectors restrict and/or constrain relative movement from between their respective devices, typically in six degrees of freedom. For example, in its connected state, the first anchor connector restricts and/or constrains relative movement between the first leaflet anchor and the center body, typically requiring them to move in sync in six degrees of freedom. Decoupling or releasing the connector connections places them in decoupled, detached, or released states, which remove the restrictions and/or constraints on in-sync (relative) movement between the respective devices (as compared with their unsevered states). The removed restrictions and/or constraints are typically in six degrees of freedom, but may be in fewer degrees of freedom.

While the term “sever” in various forms is used herein, it should be understood that in the context of this application and the claims, the term “sever,” “severable,” “severing,” etc. and similar terms encompass decoupling, disconnection, detachment, removal, etc. (even if no cutting is done).

In some implementations, the treatment/repair device 101 also includes a system or mechanism, such as a controlled tether system or mechanism or other adjustment system/mechanism. In some implementations, the system includes a respective controlled tether for each anchor, e.g., a first controlled tether 141 for the first leaflet anchor 113 and a second controlled tether 143 for the second leaflet anchor 115.

In some implementations, each controlled tether is in the form of a flexible cord (e.g., a suture). In some implementations, each controlled tether is a looped tether that extendibly fastens a primary connection (being one or more points on its respective anchor) to the center body 131 (and thereby to the delivery/implant catheter 111). In some implementations, it does so by serially extending (end-to-end) from a first-end segment at a proximal end of an implant catheter of the implant catheter system to its distal (implantation) end, through and out of the center body 131 (positioned at a distal end of the implant catheter system) to the controlled tether's respective anchor, slidably being received by and passing through (looped around) one or more anchor slidable restraints (e.g., a first anchor slidable restraint, a second anchor slidable restraint, a third anchor slidable restraint, an intermediate anchor slidable restraint and a last anchor slidable restraint) along a length of the respective anchor, back into and through and/or aside the center body and implant catheter to its proximal end, to a second-end segment at the proximal end of the implant catheter system (along with the first-end segment of the controlled tether).

In some implementations, both the first-end and second-end segments of each controlled tether are accessible by the user, either directly or using a control mechanism (e.g., one or more of a button, switch, lever, slider, knob, gear, motor, threaded component, screw, cam, gimble, etc.) for implantable devices. In use, individually and controllably the user increases the controlled tether's slack (the tether limitation) by feeding (inserting) one or both controlled tether's ends into the delivery/implant catheter 111 to increase the available tether length between the center body 131 and the anchor, allowing the respective anchor more relative separation in any direction from the center body. Individually and controllably, the user reduces each controlled tether's slack by drawing (pulling) one or both controlled tethers' ends out from the implant catheter to decrease the available tether slack between the center body and the anchor, restricting the respective anchor to less relative separation in any direction from the center body. This controllable slack provides for the anchor to move independently of the center body and implant catheter with six degrees of freedom to a controllable extent, and to be drawn back to the center body for affixing in place on the center body.

In some implementations, independent movement of the anchor is limited by a respective tether limitation that the respective anchor cannot move beyond (e.g., the tether length available for movement). That tether limitation is the degree of slack given to the respective controlled tether. In some implementations, the tether limitation changes by half the amount of controlled tether length adjusted by the user because the controlled tether loops back on itself.

Using slidable eyelet-type restraints that can be formed as part of the anchor structure, each controlled tether slidingly and/or fixedly connects to its anchor at two locations along the anchor, and slidably feeds back into the center body 131 at two locations along the center body. This two-point controlled tether connection between the anchor and center body provides better control over the relative positions of the anchor and the center body when the controlled tether slack is later removed (e.g., the controlled tether is tightened).

Prior to insertion into the subject's body, some, or all portions of the device 101, e.g., the first and second leaflet anchors 113, 115 and/or the first anchor connector 121 (in a connected state). In some implementations, the first and second leaflet anchors 113, 115 and/or the first anchor connector 121 (in a connected state) are mounted together on the distal end of the delivery/implant catheter 111. As mounted on the implant catheter prior to insertion, the structure of these portions of the device has a structural form factor sized and shaped within the limitations imposed by the delivery system for the mounted portions to simultaneously traverse the delivery system through the subject's vascular system (as a single implantable unit).

In some implementations, these portions of the device 101 can be structurally adapted to switch between a first, small-form-factor (e.g., small diameter) delivery configuration, and a second, operational configuration configured for treatment or repair operations (as depicted in FIG. 9). The delivery configuration is appropriate for the mounted portions of the device to traverse the delivery system through the subject's vascular system as a single implantable unit (optionally with a delivery configuration similar to those of other systems).

In some implementations, the implant catheter includes one or more actuators that function to actuate the device 101 from the delivery configuration to the operational configuration with the device located within the subject's heart. These actuators and their controls may be handle-type controls similar other actuators and controls (e.g., one or more of buttons, switches, levers, sliders, knobs, gears, motors, threaded components, screws, cams, gimbles, etc.) for the deployment of implants from within delivery systems.

With the delivery or implant catheter 111 extended from within steering catheter 103, pointing downstream toward the mitral valve MV, the implant catheter moves downstream into and partially or entirely through the mitral valve to the extent needed based upon the geometry of the device and the valve. If it is first delivered to the heart in a delivery configuration, either before or after passing through the mitral valve MV, the delivery/implant catheter 111 actuates the device 101 from delivery configuration to operational configuration (as depicted in FIG. 9).

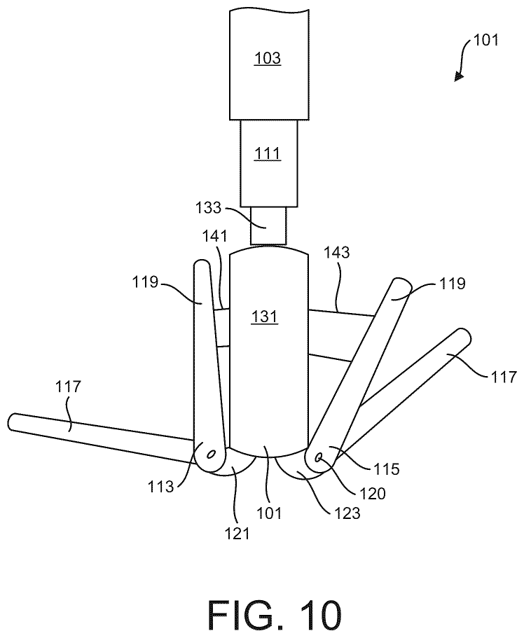

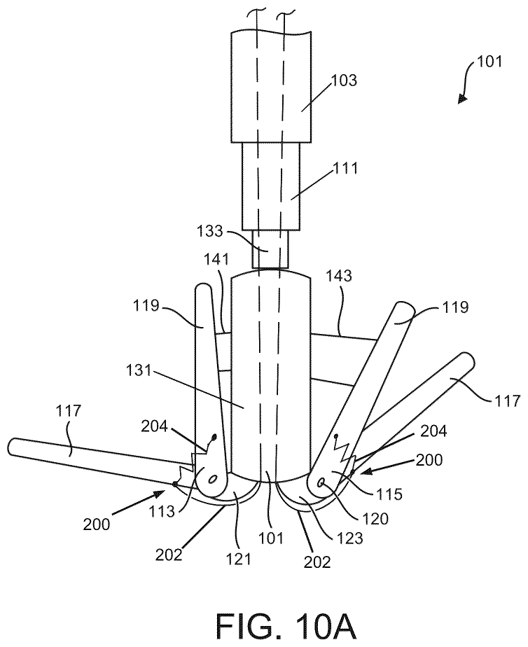

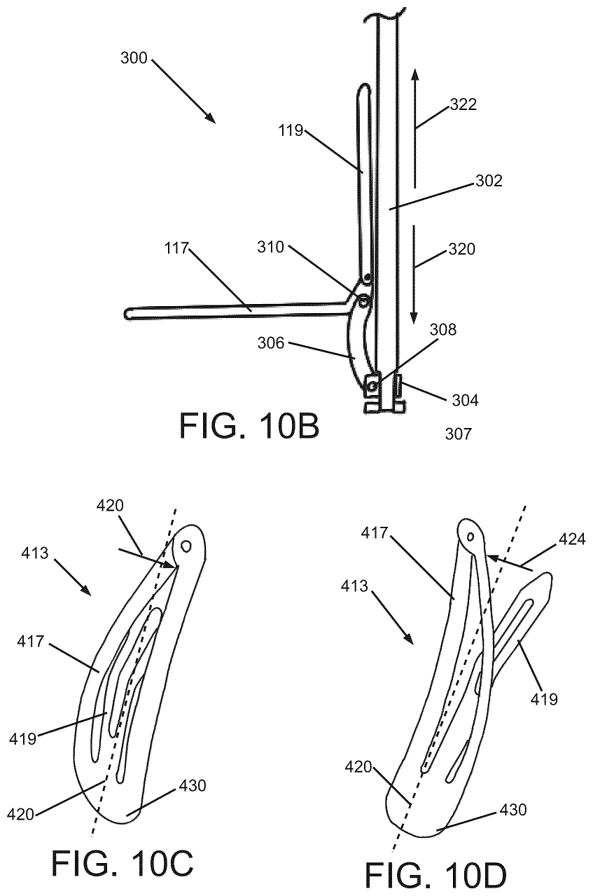

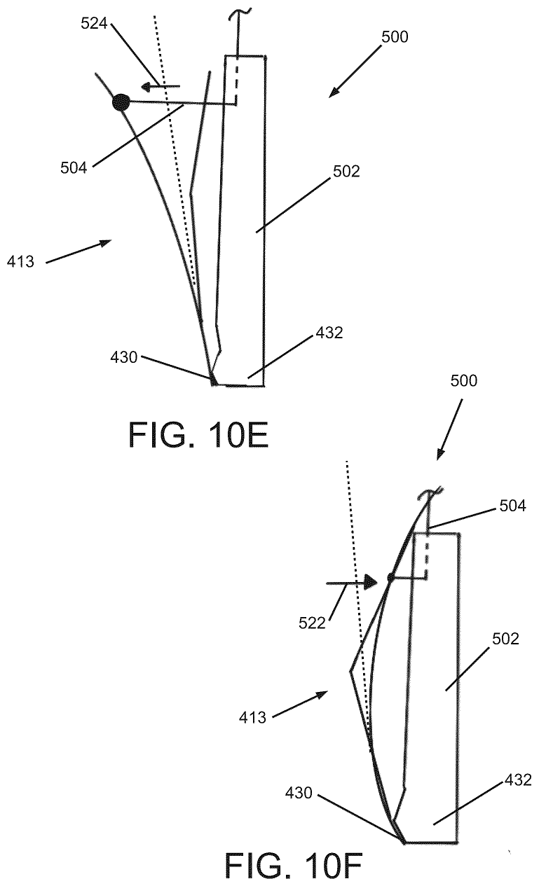

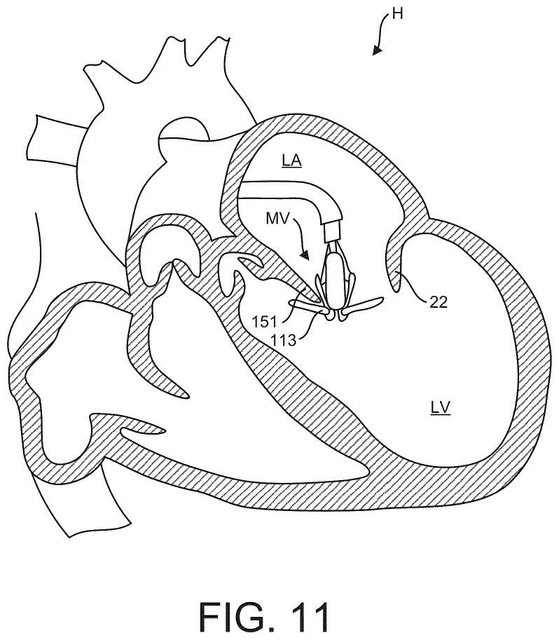

As depicted in FIGS. 10-11, with the delivery/implant catheter 111 moved downstream into and through the mitral valve MV, and the device 101 actuated into the operational configuration, the user actuates the implant catheter to move the device.

In some implementations, the implant catheter is moved, positioning the first leaflet anchor 113 at a first position within the heart, being located and oriented to affix the anchor (by clipping on) to a first portion of the edge of a first leaflet 151 requiring constraint for improved operation (e.g., a first portion of the edge of the anterior leaflet 20). The first leaflet anchor 113 is then actuated to attach it to the first portion of the edge of the first leaflet (e.g., the hinge 120 of the first leaflet anchor is actuated to anchor, e.g., clamp down, etc., on the leaflet edge, affixing it to the leaflet).

The anchoring systems, apparatuses, mechanisms, etc. for attaching the anchors to tissue herein can take a variety of different forms, e.g., clamping mechanisms, clasping mechanisms, suction mechanisms, adhering mechanisms, etc. In some implementations, the anchoring systems, apparatuses, mechanism, etc. are separate and/or independent from systems, apparatuses, mechanisms, etc. for the controlled tether. In some implementations, anchoring systems, apparatuses, mechanisms, etc. for attaching the anchors to tissue can be comparable to other anchor actuation systems, such as those that use sutures, threaded members, cams, shafts, and/or springs, etc. for controlled opening and closing of a clamp, clasp, etc. In some implementations, a clamping system, apparatus, or mechanism is used to clamp onto tissue, e.g., onto a leaflet, for anchoring.

FIG. 10A illustrates a treatment or repair device 101 that includes an example clamping mechanism 200. In some implementations, the clamping mechanism comprises a line 202, such as a suture or wire, and a biasing element 204, such as a spring, resilient member, etc. In some implementations, the biasing element 204 biases the outer paddle 117 and the inner paddle 119 toward one another. In some implementations, the biasing element 204 biases the outer paddle 117 and the inner paddle 119 away from one another.

In some implementations, such as when the biasing element biases the outer paddle 117 and the inner paddle 119 toward one another, the line 202 can be routed such that pulling on the line moves the outer paddle 117 and the inner paddle 119 away from one another to open the paddles. In some implementations, such as when the biasing element biases the outer paddle 117 and the inner paddle 119 away from one another, the line 202 can be routed such that pulling on the line outer paddle 117 and the inner paddle 119 toward one another to close the paddles.

Once in the closed position, the relative positions of the outer paddle 117 and the inner paddle 119 can optionally be locked.

FIG. 10B illustrates an example of a clamping mechanism 300. In some implementations, the clamping mechanism 300 comprises an actuation element 302 (e.g., wire, rod, tube, shaft, etc.), an optional follower 304, and a drive link 306 (e.g., strut, shaft, plate, etc.). In the illustrated example, the optional follower 304 is attached to a distal end of the actuation element 302. In some implementations, the optional follower 304 can be attached to the actuation element 302 by a fastener 307, such as a clip, nut, cap, washer, etc.

In some implementations, the drive link 306 is pivotably coupled to the optional follower 304 at a pivot axis 308. In some implementations, the follower 304 is omitted and line the drive link 306 is pivotally connected directly to the actuation element 302. In some implementations, the drive link 306 is pivotally connected to the outer paddle 117 at a pivot axis 310. In some implementations, movement of the actuation element 302 in the direction 320 moves the drive link 306, which in turn moves he outer paddle toward 117 an open condition. In some implementations, movement of the actuation element 302 in the direction 322 moves the drive link 306, which in turn moves he outer paddle toward a closed condition. An optional biasing element can bias the outer paddle 117 toward or away from the inner paddle 119. Once in the closed position, the relative positions of the outer paddle 117 and the inner paddle 119 can optionally be locked.

The first leaflet anchor 113 and the second leaflet anchor 115 can take a variety of different forms. In some implementations, the anchors 113, 115 can be configured to snap to an open position and to snap to a closed position. For example, the first leaflet anchor 113 and the second leaflet anchor 115 can be configured like a snap hair clip.

Referring to FIGS. and 10D, in some implementations, a leaflet anchor 413 includes a first arm 417 and a second arm 419. In some implementations, the first arm 417 and the second arm 419 are formed from a single piece curved and/or bent material, such as a piece of metal or plastic that has spring-like properties. In some implementations, the first arm 417 and the second arm 419 are connected together at a closed end 430.

In some implementations, the first arm 417 and/or the second arm 419 are shape set to snap to a closed configuration (FIG. 10C) and to snap to an open configuration (FIG. 10D). In some implementations, moving the first arm 417 past a center or snap-over line 420 in the direction indicated by arrow 422 causes the leaflet anchor to snap to the closed condition illustrated by FIG. 10C. In some implementations, moving the first arm 417 past the center or snap-over line 420 in the direction indicated by arrow 424 causes the leaflet anchor to snap to the open condition illustrated by FIG. 10D.

FIGS. 10E and 10F illustrate an example of a clamping mechanism 500 that uses the leaflet anchor 413 illustrated by FIGS. 10C and 10D. In some implementations, as illustrated, the clamping mechanism 500 includes a base 502 (e.g., a frame, a coaptation element, and/or a tube, etc.) an actuation element 504 (e.g., a wire, a hypotube, a flexible shaft, etc.), and the leaflet anchor 413. The closed end 430 of the leaflet anchor 413 can be fixed to or coupled to a distal end 432 of the base 502. In some implementations, the actuation element 504 can extend through the base 502 and can be releasably coupled to the first arm 417.

Referring to FIG. 10E, in some implementations, pushing the first arm 417 with the actuation element 504 past a center or snap-over line 420 in the direction indicated by arrow 524 causes the leaflet anchor 413 to snap to the open condition. Referring to FIG. 10F, in some implementations, pulling the first arm 417 with the actuation element 504 past the center or snap-over line 420 in the direction indicated by arrow 522 causes the leaflet anchor to snap to the closed condition.

In some implementations, the first leaflet anchor 113 can be a separable anchor. In some implementations, with the first leaflet anchor 113 attached to the first location on the first leaflet, the user actuates a control decoupling or detaching the first anchor connector 121 and thereby releasing the first leaflet anchor from its primary connection to the implant catheter (e.g., its connection by the first anchor connector). Therefore, the center body and the implant catheter are freed to translate and rotate together in six degrees of freedom relative to the first leaflet anchor. In some implementations, the decoupling or detachment of the separably attached first leaflet anchor 113 leaves only the first controlled tether 141 loosely connecting the first leaflet anchor 113 to the delivery/implant catheter 111. Thus, they are constrained by one another only in that they cannot separate from one another by a distance farther than is allowed by the length of the slack, which is a limitation of the first anchor tether.

In some implementations, the first anchor connector 121 provides for the delivery or implant catheter 111 and center body 131 to position the first leaflet anchor 113 in a correct position for affixing to the first leaflet 151, and then after the first leaflet anchor is affixed to the first leaflet, to decouple/detach/sever/release/unfasten the first leaflet anchor from the implant catheter, center body and second leaflet anchor 115. In some implementations, the delivery/implant catheter 111, center body 131 and second leaflet anchor 115 can then move separately from the first leaflet anchor 113, such as to position the second leaflet anchor 115 at a second position within the heart, being located and oriented to grasp a first portion of the edge of a second leaflet 153 requiring constraint for improved operation (e.g., a first portion of the edge of the posterior leaflet 22). The second leaflet anchor 115 can be a separable anchor.

In some implementations, the user then gives the first controlled tether 141 adequate slack to allow the implant catheter to freely move (with the second leaflet anchor 115 still attached) to a second location (such as at a location on a second leaflet 153 requiring constraint) without pulling the first leaflet anchor 113 and the first leaflet 151 along with it, and without being detrimentally pulled and tugged upon by the motion of the first leaflet due to the operation of the heart.

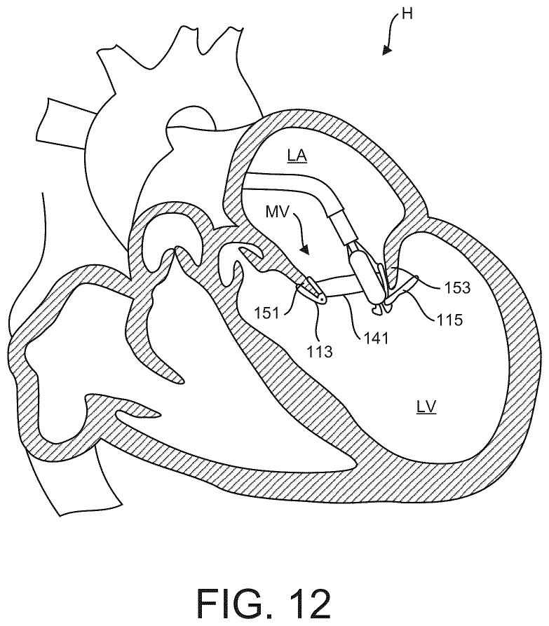

In some implementations, as depicted in FIG. 12, with the delivery or implant catheter 111 extending through the mitral valve MV, the device 101 is actuated into an operational configuration. In some implementations, the first leaflet anchor 113 is affixed to the first leaflet and the first 151, anchor connector 121 is decoupled/detached/severed/unfastened/released to free the first leaflet anchor from direct attachment to the implant catheter (though it may still be indirectly attached via tethered connections to other components that may still be directly attached to the implant catheter). In some implementations, the user actuates the implant catheter to move the implant catheter and its attached portions of the device, positioning the second leaflet anchor at a second position within the heart, being located and oriented to grasp a first portion of the edge of a second leaflet 153 requiring constraint for improved operation (e.g., a first portion of the edge of the posterior leaflet 22). This positioning occurs without pulling the first leaflet anchor and the first leaflet to the second leaflet.

If the first controlled tether slack is not already adequate, the user can controllably release (lengthen) slack on the first controlled tether 141 to provide for this movement without detrimental resistance from the first leaflet. This can be done by the user releasing slack on an end of the controlled tether suture at the proximal end of the implant catheter, as for other transvascular mechanisms having wires, chords, lines, etc. for control. The implant catheter can then be actuated to position the second leaflet anchor 115 at the second position. The second leaflet anchor 115 can then be actuated to attach it to the first portion of the edge of the second leaflet (e.g., the second leaflet anchor is actuated to clamp down on the leaflet edge to affix it to the leaflet).

In some implementations, with the second leaflet anchor 115 attached to the first location on the second leaflet 153, the user actuates a control causing the second anchor connector 123 to decouple/detach/sever/unfasten and thereby release the second leaflet anchor from the implant catheter. Therefore, the delivery/implant catheter 111 and center body 131 are freed to translate and rotate in six degrees of freedom relative to the second leaflet anchor 115.

The decoupling or detachment of the separably attached second leaflet anchor 115 leaves only the second controlled tether 143 loosely connecting the second leaflet anchor to the implant catheter. Thus, they are constrained by one another only in that they cannot separate from one another by a distance farther than is allowed by the length of the slack, which is a limitation of the first anchor tether.

In some implementations, the second anchor connector 123 provides for the delivery or implant catheter 111 to first position the second leaflet anchor 115 for affixing to a second leaflet 153, and then, after the second leaflet anchor is affixed to the second leaflet, to decouple/detach/unfasten/sever and thereby release the second leaflet anchor 115 from attachment directly to the implant catheter (within the limits allowed by the slack in the second controlled tether 143). The delivery/implant catheter 111 can then move separately from the second leaflet anchor 115, such as to position the implant catheter and center body 131 at location central to the leaflets.

In some implementations, the system (e.g., controlled tether system, tether system, coaptation system, actuation system, connection system, control system, etc.) is configured such that a user can give the second controlled tether 143 adequate slack to allow the implant catheter to freely move to a location between the leaflets without pulling either the first leaflet anchor 113 and the first leaflet 151, or the second leaflet anchor 115 and second leaflet 153, along with it (and without being detrimentally pulled and tugged upon by the heartbeat-driven motion of the first and second leaflets due to the heart activity).

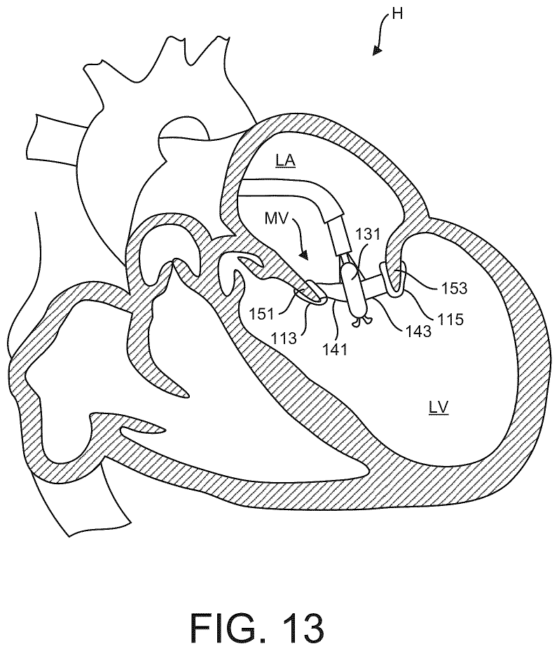

In some implementations, as depicted in FIG. 13, the delivery or implant catheter 111 and center body 131 are configured such that they can be manipulated by a user to be positioned at a coaptation location between the leaflets. This positioning occurs without pulling the second leaflet anchor and the second leaflet to the coaptation location. Once the delivery/implant catheter 111 is at the coaptation location, one or more controls (e.g., handle controls, buttons, knobs, switches, sliders, gears, other controls, etc.) can be actuated at the proximal end of the delivery system, e.g., on a handle. In some implementations, the controls respectively tighten the first and second controlled tethers 141, 143, to shorten and remove their respective slack. The controls can be configured to controllably tighten one controlled tether at a time, or all controlled tethers at once.

While the location of the distal end of the delivery/implant catheter 111 will establish a location towards which the first and second leaflets 151, 153 are drawn, the leaflet structural forces and the dynamic blood flow forces from each leaflet will determine the actual location where the resulting device 101 (e.g., the pair of anchors and the center body) will end up. Based on the attachment of the respective controlled tethers, each anchor will define an opposing surface that can be drawn closer and/or into contact with the center body.

With the first and second leaflet anchors 113, 115 connected to the delivery/implant catheter 111 and center body 131 (e.g., in contact with, closer to, etc.), and thereby connected to one another, an anchor-lock locking mechanism locks the anchors into position (or region) relative to the center body and one another, thereby constraining relative movements of the first leaflet anchor, the second leaflet anchor, and the center body. In some examples, this entails holding the anchors and center body in contact and stationary (e.g., completely stationary, mostly stationary, portions (e.g., connected surfaces) are completely stationary while other portions/extensions may move, etc.) relative to each other. A variety of structural/mechanical locking mechanisms can be used as the anchor-lock to effectuate this locking of the anchors. For example, the center body can be provided with one or more locks that structurally mate and interlock with each tethered anchor to lock them in position.

In some implementations, the first and second anchor connectors 121, 123 can be configured to act as all or part of the anchor-lock locking mechanism. In some implementations, the anchors can be configured with locking mechanisms that lock directly to one another to form the anchor-lock. In some implementations, segments of the first and second controlled tethers 141, 143 can be cut off and used as part of the anchor-lock to lock the first and second leaflet anchors 113, 115 into position with respect to the center body and/or one another. In some implementations, distal segments of the tightened controlled tethers are actuated (e.g., pulled), thereby tensioning or pulling on the anchors to cinch them toward the center body (and thereby moving the anchors together toward one another and/or into contact with each other and/or with another portion of the device, e.g., a center body, spacer, etc.). In some implementations, the actuated controlled tethers can be locked together to lock the anchors into their relative positions, and then the distal segments of the controlled tethers can be decoupled/detached/severed/cut/etc. from their respective remaining (proximal) controlled tether segments (and from the implant catheter) to permanently leave the distal segments holding the anchors in their fully connected (e.g., in contact) relative positions. The term “cinch” includes pulling and/or tightening.

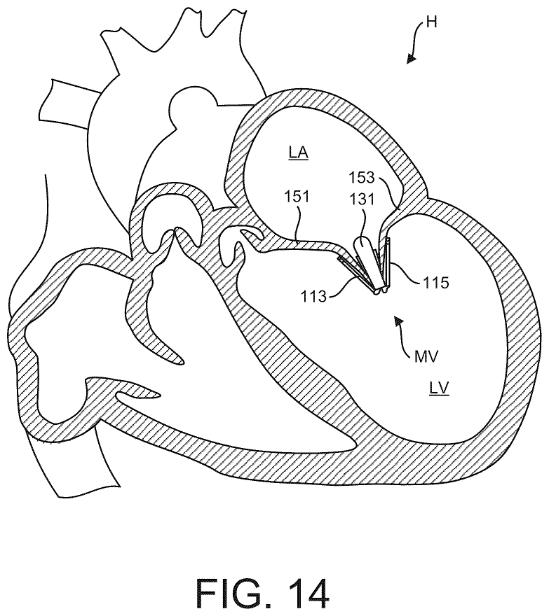

In some implementations, once the first and second leaflet anchors 113, 115 are locked into position with respect to the center body 131 and one another, the first and second controlled tethers 141, 143 and the delivery/implant catheter 111 are disconnected from the device 101 and removed from the subject's body (see, FIG. 14). In some implementations, the second-end segment of each controlled tether is freed to advance into the delivery/implant catheter (to pass up through the anchor slidable restraints and then back down the implant catheter) while the first-end segment is continuously pulled from the implant catheter until the full length of the controlled tether comes out of the delivery/implant catheter. In some implementations, where distal segments of the controlled tethers were cut from proximal segments, each proximal segment may need to be individually pulled from the implant catheter.

With all of the controlled tethers (or segments thereof) removed, a control can be actuated causing the center-body connector 133 to decouple/detach/sever/unfasten, releasing the center body from the delivery or implant catheter (if the center body is the last component released from the implant catheter, the entire device may then be decoupled or released from the delivery or implant catheter). When fully released, the center body 131 and connected locked anchors are completely freed to translate and rotate in six degrees of freedom with the leaflets. Finally, the delivery system can be removed from the subject.

By making one or more inserted anchors separable from one another (and the rest of the implant) prior to leaflet attachment, anchors can be attached to leaflets without resistance from other previously attached anchors. The device can be placed without suffering the difficulties that may be caused by leaflets having excessive spacing and movement relative to one another.

The devices and/or systems herein can comprise a wide variety of simultaneous, center-coupled implementations, some of which are expressly described herein. To the extent appropriate (e.g., can be implemented), these are also envisioned for use with other implementations. For example, other implementations can include combinations or sub-combinations of the features and components disclosed for the implementations below.

In some simultaneously inserted center-coupled implementations, the device can include three or more anchors. In some implementations, a treatment/repair device is provided with three anchors, at least two of which are separably attached to a center body. This is particularly advantageous when implanted to improve the operation of a valve with three leaflets, such as the tricuspid, aortic and pulmonary valves.

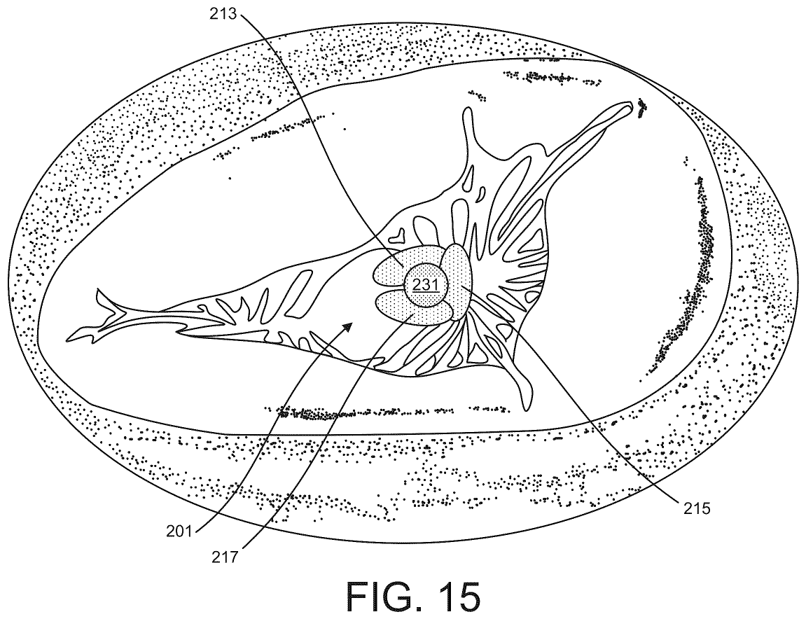

In FIGS. 15-20, an example simultaneously inserted center-coupled implementation with three anchors is depicted during implantation in a tricuspid valve TV. Unless indicated otherwise, elements of the device are substantively the same as similarly named items in the earlier referenced implementations. Where possible, reference numbers are incremented by 100 from the related center-coupled implementation.

In some implementations, device 201 (e.g., treatment device, repair device, valve treatment device, valve repair device, implantable device, implant, etc.) is delivered through the vascular system to the tricuspid valve TV by a delivery system for valve treatment or repair having similarities to the center-coupled implementation. An implant catheter is inserted into the subject's body and extended downstream toward the tricuspid valve. At its distal end, the implant catheter carries the device 201, which includes three anchors, a first leaflet anchor 213, a second leaflet anchor 215, and a third leaflet anchor 217, each with a leaflet-attachment mechanism in the form of an actuatable clip configured to clip on to the edge of a leaflet that requires constraint for improved valve operation (e.g., a third leaflet-attachment mechanism).

In some implementations, the device 201 also includes a connector for each anchor, e.g., a first anchor connector, a second anchor connector, and/or a third anchor connector. In some implementations, the device is further provided with an optional center body 231, and a center-body connector rigidly fastening the center body to the implant catheter. In some implementations, the first, second and third anchor connectors are configured to fixedly and/or rigidly fasten their respective anchors to the center body 231 in six degrees of freedom, and thereby indirectly rigidly fasten them to the implant catheter via the center body.

The device 201 also includes a first controlled tether 241, a second controlled tether 243, and/or a third controlled tether 245, each in the form of a flexible suture. Each controlled tether fastens its respective anchor to the center body 231, and thereby the implant catheter. Each controlled tether can controllably be given slack for the anchor to move independently of the center body and implant catheter, being limited by a respective tether limitation (slack), being the tether length available for movement. Each controlled tether slidingly and/or fixedly connects to its anchor at two locations along the anchor, and to the center body 231 at two locations along the center body.

In some implementations, each controlled tether is a looped tether that extendibly fastens a primary connection (being one or more points on its respective anchor) to the center body 131 (and thereby to the delivery/implant catheter 111). It can be done by serially extending (end-to-end) from a first end (e.g., a third controlled-tether first-end segment) at a proximal end of the implant catheter system to its distal (implantation) end, through and out of the center body 131 (positioned at a distal end of the implant catheter system) to the controlled tether's respective anchor, slidably being received by and passing through (looped around) one or more slidable restraints along a length of the respective anchor, back into and through and/or aside the center body and implant catheter to its proximal end, to a second end (e.g., a third controlled-tether second-end segment) at the proximal end of the implant catheter system (along with the first-end segment of the controlled tether).

The device 201 may optionally be structurally configured with a first, small-form-factor delivery configuration and a second, operational configuration configured for valve treatment or repair operations. In some implementations, a small-form-factor, side-by-side serial delivery configuration (e.g., spaced at 120° angles) might be slim enough to pass through a transvascular catheter system. In others, a more complex delivery configuration might be necessary.

With the implant catheter extended from within steering catheter system, pointing downstream toward the tricuspid valve TV, the implant catheter moves downstream into and partially or entirely through the tricuspid valve to the extent needed based upon the geometry of the device and the valve. If it is first delivered to the heart in a delivery configuration, either before or after passing through the tricuspid valve TV, the implant catheter can actuate the device 201 from a delivery configuration to an operational configuration.

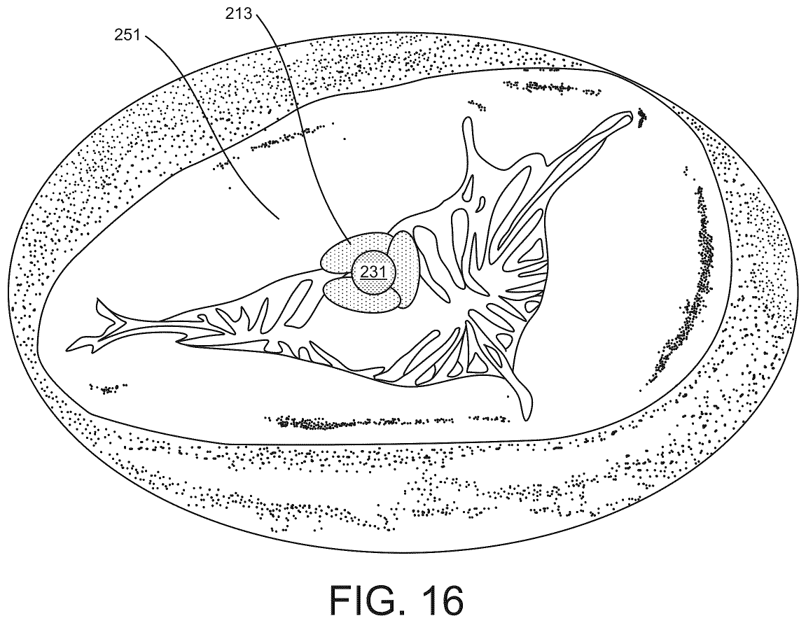

As depicted in FIG. 16, with the implant catheter moved downstream into and through the tricuspid valve TV, and the device 201 actuated into the operational configuration, the implant catheter can be actuated to move the implant catheter and the device, positioning the first leaflet anchor 213 at a first position within the heart, being located and oriented to grasp a first portion of the edge of a first leaflet 251 requiring constraint for improved operation. The first leaflet anchor 213 can then be actuated to attach it to the first portion of the edge of a first leaflet.

With the first leaflet anchor 213 attached to the first leaflet 251, the first anchor connector can be decoupled/detached/severed/unfastened to release the first leaflet anchor from the implant catheter. The center body 231 and implant catheter can be freed to translate and rotate together relative to the first leaflet anchor 213. The decoupling or detachment of the separably attached first leaflet anchor 213 leaves only the first controlled tether 241 loosely connecting the first leaflet anchor 213 to the implant catheter.

The first anchor connector provides for the implant catheter and center body 231 to position the first leaflet anchor 213 in a correct position for affixing to the first leaflet 251, and then after the first leaflet anchor is affixed to the first leaflet, to unfasten and release the first leaflet anchor from the implant catheter, center body, second leaflet anchor 215 and third leaflet anchor 217. The implant catheter, center body 231, second leaflet anchor 215 and third leaflet anchor 217 can then move separately from the first leaflet anchor 213 to position the second leaflet anchor 215 at a second position within the heart to grasp a first portion of the edge of a second leaflet 253 requiring constraint for improved operation.

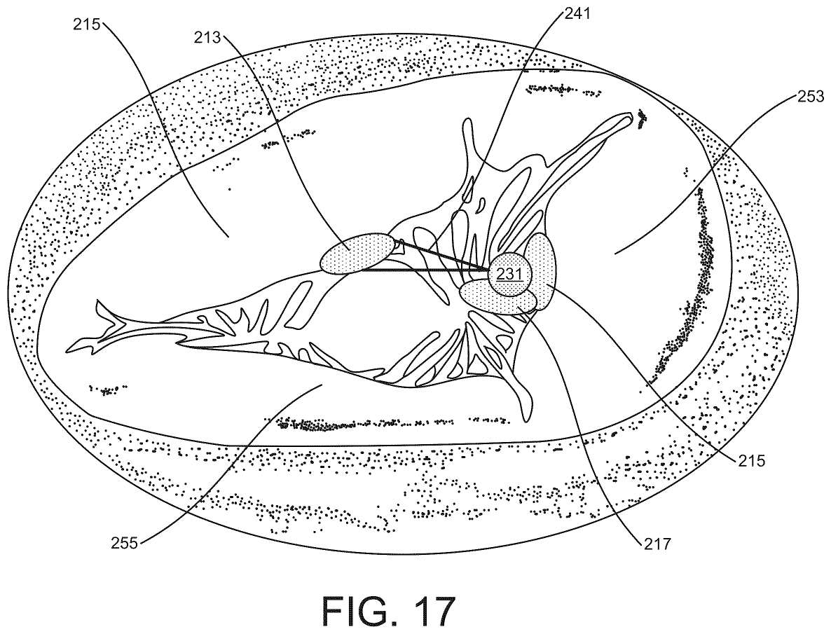

As depicted in FIG. 17, the first controlled tether 241 can be given adequate slack to allow the implant catheter to freely move to a second location at a second leaflet 253 without pulling the first leaflet anchor 213 and the first leaflet 251 along with it. The implant catheter and the attached portions of the device can be moved to a second position within the heart, positioning (locating and orienting) the second leaflet anchor to grasp a first portion of the edge of a second leaflet 253 requiring constraint for improved operation.

If the first controlled-tether slack is not already adequate, the slack can be controllably released on the first controlled tether 241 to provide for this movement without detrimental resistance from the first leaflet. The implant catheter can be actuated to position the second leaflet anchor 215 at the second position. The second leaflet anchor 215 can be actuated to attach it to the first portion of the edge of the second leaflet.

With the second leaflet anchor 215 attached to the second leaflet 253, the second anchor connector can be decoupled/detached/severed/unfastened to release the second leaflet anchor from the implant catheter. In some implementations, the decoupling or detachment of the separably attached second leaflet anchor 115 leaves only the second controlled tether 143 loosely connecting the second leaflet anchor to the implant catheter.

In some implementations, the second anchor connector provides for the implant catheter and center body 231 to position the second leaflet anchor 215 in a correct position for affixing to a second leaflet 253, and then after the second leaflet anchor is affixed to the second leaflet, to unfasten and release the second leaflet anchor from the implant catheter, center body 231 and third leaflet anchor 217. The implant catheter, center body and third leaflet anchor can then move separately from the second leaflet anchor 215 and the first leaflet anchor 213 to position the at a third position within the heart to grasp a first portion of the edge of a third leaflet 255 requiring constraint for improved operation.

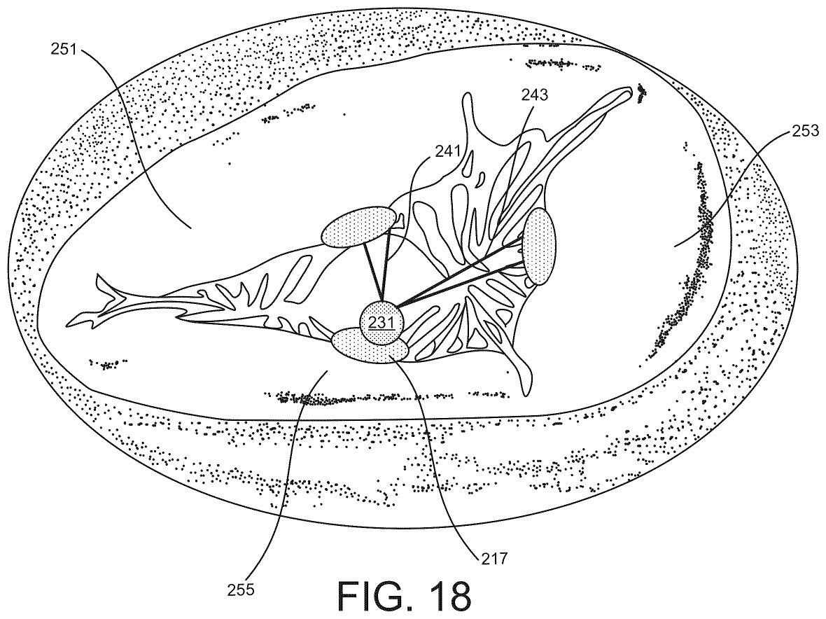

As depicted in FIG. 18, the second controlled tether 243 can be given adequate slack to allow the implant catheter to freely move to a third location at a third leaflet 255 without pulling the first leaflet anchor 213 and the first leaflet 251, or the second leaflet anchor 215 and the second leaflet 253, along with it. The implant catheter and portions of the device can be moved to a third position within the heart, positioning (locating and orienting) the third leaflet anchor to grasp a first portion of the edge of a third leaflet 255 requiring constraint for improved operation.

If the first controlled-tether slack and second controlled-tether slack are not already adequate, slack on the first controlled tether 241 and second controlled tether 243 can be controllably released to provide for this movement without substantial detrimental resistance from the second leaflet 253. The implant catheter can be actuated to position the third leaflet anchor 217 at the third position. The third leaflet anchor 217 can be actuated to attach it to the first portion of the edge of the third leaflet.

With the third leaflet anchor 217 attached to the third leaflet 255, the third anchor connector can be decoupled/detached/severed/unfastened to release the third leaflet anchor from the implant catheter. In some implementations, the decoupling or detachment of the separable third leaflet anchor leaves only the third controlled tether 245 loosely connecting the third leaflet anchor to the implant catheter.

In some implementations, the third anchor connector provides for the implant catheter to first position the third leaflet anchor 217 for affixing to a third leaflet 255, and then, after the third leaflet anchor is affixed to the third leaflet, to decouple/detach/sever/unfasten and thereby release the third leaflet anchor 217 from the implant catheter (within the limits allowed by the slack in the third controlled tether 245). The implant catheter can then move separately from the third leaflet anchor 217, such as to position the implant catheter and center body 231 at location central to the leaflets.

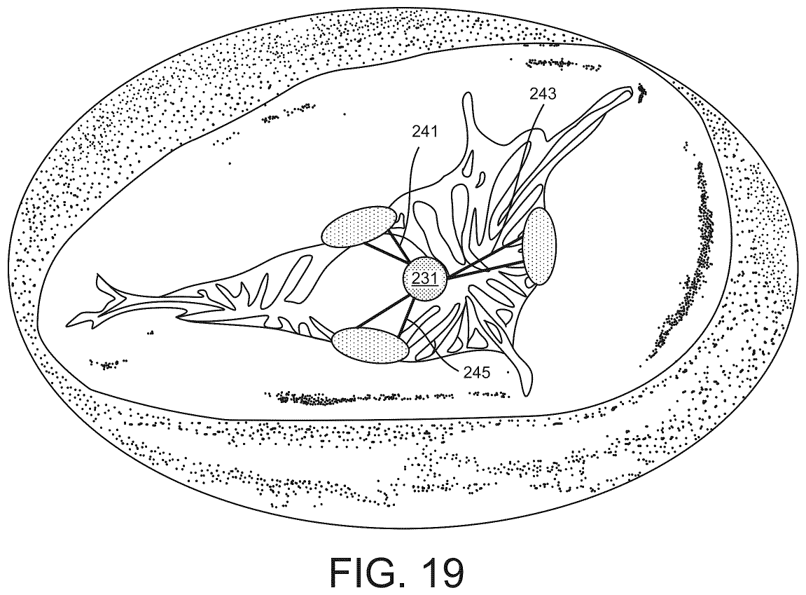

As depicted in FIG. 19, the third controlled tether 245 can be given adequate slack to allow the implant catheter to freely move to a location (e.g., a central location, a commissural location, etc.) between the leaflets without pulling either the first leaflet anchor 213 and the first leaflet 251, the second leaflet anchor 215 and second leaflet 253, or the third leaflet anchor 217 and third leaflet 255 along with it.

In some implementations, the implant catheter and center body 231 are then positioned at a coaptation location between the leaflets (e.g., a central location, a commissural location, etc.). Once the implant catheter is at the coaptation location, the user actuates controls (e.g., one or more handle controls, buttons, knobs, switches, touchscreens, etc.) to respectively tighten the first, second and third controlled tethers 241, 243, 245, to shorten and remove (or reduce) their respective slack. The leaflet structural forces and the dynamic blood flow forces from each leaflet will determine the location where the resulting device 201 (e.g., the connected anchors and the center body) will end up.

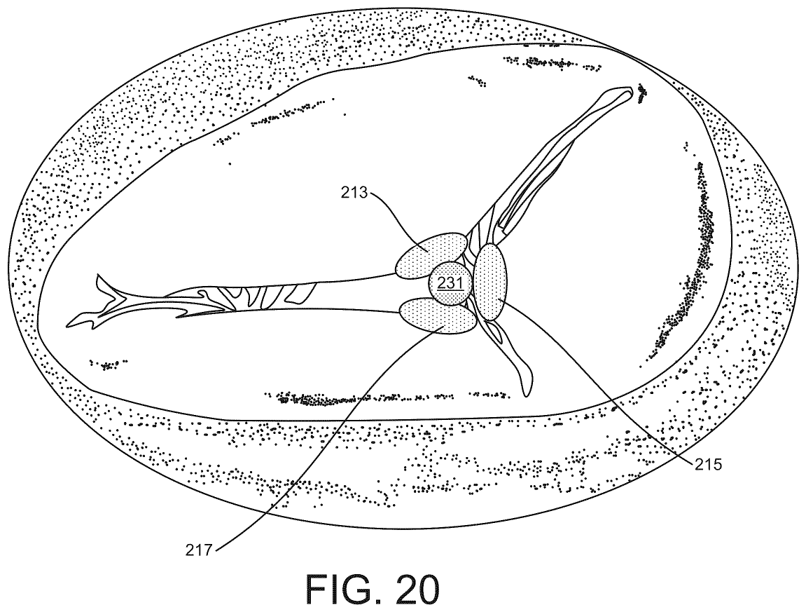

As depicted in FIG. 20, with the first, second and third leaflet anchors 213, 215, 217 connected to (e.g., in contact with) the center body 231. The anchors can be locked into position (or relative regions) with respect to the center body and one another. A variety of mechanisms can be used to effectuate this locking of the anchors. For example, as part of the anchor-lock, the center body can be provided with one or more mechanical locks (e.g., clips, clamps, portions that structurally mate, interlocking portions, etc.) to lock each tethered anchor in position and/or relative to each other. In some implementations, these are locked into contact and/or stationary relative to each other (e.g., completely stationary, mostly stationary, with portions that are stationary (e.g., contact surfaces) and other portions (e.g., extensions) that can move somewhat, etc.)

Optionally, the anchor connectors can be configured to act as all or a part of the anchor-lock. Optionally, the anchors can be configured with mechanical locks that lock directly to one another as part of the anchor-lock. Optionally, distal segments of the first, second and third controlled tethers may be cut off and used as part of the anchor-lock to lock the first, second and third leaflet anchors 213, 215, 217 into position with respect to the center body and/or one another. In some implementations, distal segments of the tightened controlled tethers are cinched together toward the center body and one another, and then decoupled/detached/severed/released from their respective remaining proximal segments (and the implant catheter) to permanently hold the anchors in their final connected positions.

In some implementations, with the first, second and third leaflet anchors 213, 215, 217 locked into position with respect to the center body 231 and one another, the first, second and third controlled tethers 241, 243, 245 and the implant catheter are disconnected from the device 201 and removed from the subject's body (see, FIG. 14). More particularly, the second-end segment of each controlled tether is freed to advance into the implant catheter (to pass up through the anchor slidable restraints and then back down the implant catheter) while the first-end segment is continuously pulled from the implant catheter until the full length of the controlled tether comes out of the implant catheter.

In some implementations, once the first, second and third leaflet anchors 213, 215, 217 are locked into position with respect to the center body 231 and one another, the implant catheter is disconnected from the device 201 and removed from the subject's body. Therefore, the center body 231 and the three attached anchors are freed to translate and rotate with the valves in six degrees of freedom.

By making one or more inserted anchors separable from one another (and the rest of the implant) prior to leaflet attachment, anchors can be attached to leaflets without resistance from other previously attached anchors. The device was placed without suffering the difficulties caused by leaflets having excessive spacing and movement.

While the described 3-anchor implementation connects three leaflets on a tricuspid valve, other scenarios are also within the scope of this disclosure. For example, on a mitral valve (or any three-leaflet valve), two of the three anchors can be attached to a first leaflet while the third leaflet anchor is attached to a second leaflet. During the process of implanting, the anchors can be attached alternately to the two leaflets (e.g., to the first leaflet, then to the second, then again to the first), or the single-anchor leaflet can be attached to its anchor either before or after the leaflet with two anchors. Advantageously, this can offer additional structural support to a damaged leaflet at risk of further structural failure, and/or better attachment to a leaflet having characteristics indicating poor anchor-attachment characteristics. In some implementations, two anchors on one leaflet may be placed adjacent to each other on the leaflet (e.g., in contact, etc.) or may be placed spaced apart along the leaflet.

Using two anchors on one leaflet (which can be done with any devices herein having only two anchors, devices having three anchors, and/or devices having more anchors) and bringing them closer together may plicate a portion of the leaflet, which could help treat various leaflet issues, e.g., prolapse, etc.

With reference again to FIGS. 9-14, in some simultaneously inserted center-coupled implementations, the second leaflet anchor 115 can be inseparably connected to the center body 131 (e.g., connected to the center body without the use of a controllable anchor connector such that the second leaflet anchor cannot be controllably separated from the center body while within the body of a subject). In some implementations, this inseparably connected anchor is still separably connected to the delivery/implant catheter 111, in that the center body 131 is separably connected to the implant catheter. In some implementations, the first leaflet anchor 113 is separably connected to the center body 131 by a first anchor connector 121.

In some implementations, the first anchor connector 121 still provides for the delivery or implant catheter 111 to position the first leaflet anchor 113 in a correct position for affixing to the first leaflet 151, and then after the first leaflet anchor is affixed to the first leaflet 151, to decouple/detach/sever/unfasten and thereby release the first leaflet anchor from the implant catheter, center body 131 and second leaflet anchor. The delivery/implant catheter 111 and center body 131 can then separate and move away from the first leaflet anchor 113, such as to position the second leaflet anchor 115 at a second position within the heart, where it can be attached to the first portion of the edge of the second leaflet 153.

In some implementations, such as with the second leaflet anchor 115 attached by a non-severable-connector (e.g., a connector that is not a severable connector), the location of the second leaflet 153 is used as a coaptation location (to the extent that the catheter remains rigidly in place). In some implementations (e.g., as described previously), the leaflet structural forces and the dynamic blood flow forces from each leaflet will determine the actual location where the resulting device 101 will end up once the anchors are locked into position with respect to the center body (and one another), and the delivery/implant catheter 111 is disconnected from the device 101.

Center-coupled implementations with multiple or all anchors separable may provide the user with a greater flexibility in the choice of which anchor attaches to which leaflet. Nevertheless, implementations with one or more anchors that are inseparable can provide benefits in the form of procedural simplicity of fewer connectors to decouple/detach/sever and fewer anchors to lock into position (with respect to the center body). These may also benefit from the cost and size advantages of a less complicated structure.

When combined three or more anchors, one anchor might be inseparable while the others are separable. In some implementations, two or more anchors might be inseparable while at least one is separable. In some implementations, greater flexibility during use is emphasized, e.g., with two separable anchors. In some implementations, the cost and size of a less complicated structure can be emphasized, e.g., with non or only one anchor separable.

In some simultaneously inserted center-coupled implementations, the system (e.g., controlled tether system, tether system, etc.) includes one or more multi-anchor controlled tethers that each have an anchor-tether section with a plurality of anchor-tether segments (e.g., a first anchor-tether segment, a second anchor-tether segment and a third anchor-tether segment), each anchor-tether segment extending from an anchor-tether segment connection point (or a plurality of anchor-tether segment connection points) on the anchor-tether section to a respective one of the anchors having anchor connectors (thereby joining the anchor-tether segments). In forming the anchor-tether section, the anchor-tether segments can be connected at the connection points with connection mechanisms, or they can be unitary (e.g., form a unitary tether that is unitary at their connection points in their original formation). The connection points might typically be located at or within the center body prior to and/or after the anchor connectors are decoupled/detached/severed.

In some implementations, the system is a controlled tether system that includes an extension-tether section that is configured to extendibly fasten a primary connection point of the anchor-tether segments (and thereby to connect to their respective anchors) to the delivery/implant catheter 111 (optionally through center body 131). In some implementations, to removably make this connection, the extension-tether section serially extends (end-to-end) from an extension-tether first-end segment at a proximal end of the implant catheter system (protruding from a proximal, user end of the implant catheter), down along (through or aside) the implant catheter to its distal (implantation) end, into the center body 131 positioned at a distal end of the implant catheter, slidably received by and connecting to (loopingly passing around/through) the primary connection point of the anchor-tether segments, back out of the center body, back along (through or aside) the implant catheter to its proximal end, to an extension-tether second-end segment at the proximal end of the implant catheter system (protruding from the proximal, user end of the implant catheter system along with the extension-tether first-end segment of the extension-tether section).

In some implementations, the connection is formed between the extension-tether section and the primary connection point of the anchor-tether segments by an intermediate segment of the extension-tether section looping around the primary connection point of the interconnected anchor-tether segments. This configuration provides for the extension-tether section to be slidingly disconnected from the interconnected anchor-tether segments (by pulling on one end of the extension-tether section) once the anchor-lock constrains relative movement of the connected leaflet anchors.

In some simultaneously inserted center-coupled implementations, the controlled tethers do not extend end-to-end from the catheter proximal end to their anchors and back. Instead, each controlled tether (e.g., the first controlled tether) extends end-to-end from a first tether end (e.g., a first controlled-tether first-end segment) at a proximal end of the implant catheter system (protruding from a proximal, user end of the implant catheter), down along (through or aside) the implant catheter to its distal (implantation) end, through and out of the center body 131 positioned at (and separably connected to) a distal end of the implant catheter system, to the controlled tether's respective anchor.

In some implementations, at the controlled tether's respective anchor, the controlled tether is affixed to the controlled tether's respective anchor. Thus, the first controlled tether serially extends from a first controlled-tether first-end segment at a proximal end of the implant catheter system, through a center-body slidable restraint on a center body separably attached to a distal end of the implant catheter system (by a center-body connector), to a first controlled-tether second-end segment affixed to the controlled tether's respective anchor.

In some implementations, at the controlled tether's respective anchor, it is slidably received by and passes through one or more slidable restraints along a length of the respective anchor, and then back to the center body, to which it fixedly connects. In some implementations, the first controlled tether serially extends from a first controlled-tether first-end segment at a proximal end of the implant catheter system, through a center-body slidable restraint on a center body separably attached to a distal end of the implant catheter system (by a center-body connector), through a slidable restraint on the controlled tether's respective anchor (e.g., a first slidable restraint on a first leaflet anchor, back to a first controlled-tether second-end segment affixed to the center body.

In some implementations, each controlled tether can slidingly connect to its respective anchor at one or more locations along the anchor. In some implementations, each tether slidingly connects to the center body 131 at one location along the center body, and to the respective anchor at two locations (as depicted in FIGS. 12-13). This provides a two-point tether connection between each respective anchor and the center body to limit the orientation of the anchor when connected (e.g., when in contact with each other and/or with the center body).

In some implementations, while the anchor-affixed and center-body-affixed versions reduce both the overall required length of tether required, and the total number of tether ends extending from the proximal end of the implant catheter, some form of tether severing mechanism (e.g., a tether cutter) may be needed to separate one or more tether segments at the distal end of the controlled tether. The severing of the controlled tethers leaves one or more proximal tether segments for removal from the subject after implantation is complete. The tether cutter can be configured to individually cut each controlled tether, or it may be configured to cut all controlled tethers (that need to be cut) at once.

In some simultaneously inserted center-coupled implementations, rather than being in the form of a fully flexible cord such as a suture, one or more of the controlled tethers can be in a less flexible form, such as a mechanism or shaft having its bending and torsional flexibility selectively established to allow more flexibility in some degrees of freedom than in others. While limiting free independent movement of the anchors during the relevant periods of implantation, this form of controlled tether might provide some normalizing force to limit the movement and orientation of the anchors with respect to one another and the center body to limit complications from the ebb and flow of the blood during implantation. In some implementations, slack describes the extent to which the controlled tether can flexibly allow movement within its moveable degrees of freedom.

In some simultaneously inserted center-coupled implementations, the anchor is a T-shaped toggle anchor.

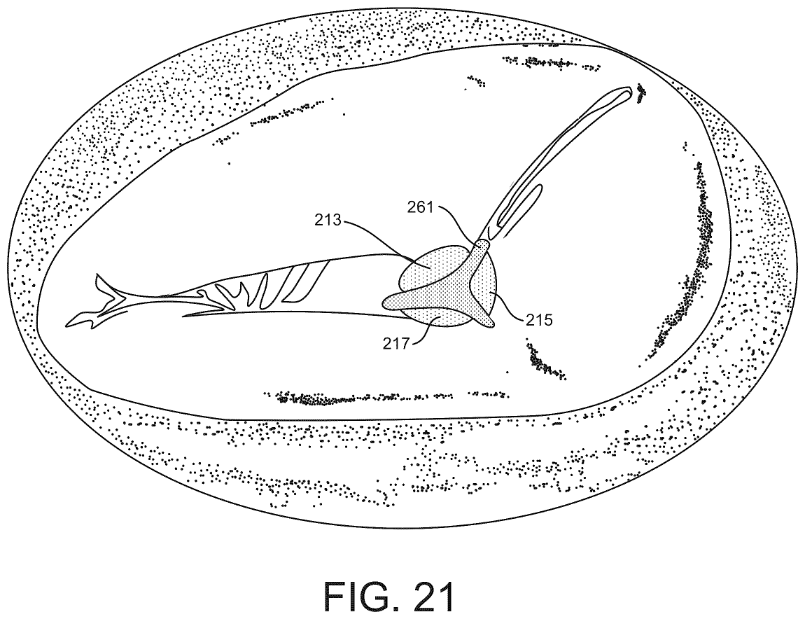

With reference to FIGS. 20-21, while center bodies of various implementations can be provided with a coaptation element or spacer in a variety of forms, e.g., such as a tubular braided wire formed of a nitinol mesh, a molded component, etc. In some center-coupled implementations, the center body 261 can be a spacer. In some implementations, the spacer can be a resilient spacer, e.g., a spacer formed of a pliant body (material and structure) having a functionally low pliant compressibility with respect to obstructed blood flow, etc. This pliant body can be made of a variety of materials, e.g., a rubber-like material and/or material structure (e.g., combination of materials), having elastomeric properties providing a viscous-like behavior of low rigidity/high fluidity that leads to deformation rather than compression under a compressive load, wherein the body substantially resumes its original shape when a deforming force is removed). As such, this resilient spacer can be formed as a relatively incompressible, pliant, unitary body that is moldable under compression without significant loss of volume. Exemplary materials can include elastomers such as a medical grade silicone, such as would be appropriate for cosmetic medical use, e.g., polymers that are non-reactive, flexible, temperature-resistant, water-resistant, and biocompatible.

A functionally low pliant compressibility with respect to obstructed blood flow of a pliant body can comprise a combination of an adequate level of pliancy, and a low enough level of compressibility, to provide for a medically relevant dimensional expansion of the pliant body into the blood flow in response to a functionally available level of force from the mechanism moving the anchors into contact with the spacer (or another type of center body). In some implementations, this can be a force leading to a dimensional expansion that has a medically significant effect on the regurgitation of blood when the pliant body is compressed with a force available from the system and/or mechanism for moving the anchors into contact with and compressing the spacer.

In some implementations, when compressed with a force between opposing surfaces of the anchors, the resilient spacer is pliant enough to conform to the shape of the anchors, and it is incompressible enough to dimensionally expand to fill in gaps therebetween and (generally) protrude out from between the anchors to the extent the coaptation forces are at a high enough level. This expansion both compliantly maintains the relative positions of the anchors, and typically provides a flow-blocking obstruction to prevent regurgitation through the valve.

In some implementations, the pliability of the resilient spacer also provides for the resilient spacer to conformingly hold the anchors in positions relative to one another such that they have a less-than-rigid connection to one another. Therefore, the anchors can move relative to one another in response to forces applied to them by the leaflets and blood flow. Thus, the resilient spacer center body may provide for connected anchors (e.g., anchors in contact with the spacer, center body, etc.) to move relative to one another, thereby adapting to the blood flow and natural movement of the leaflets during the cardiac cycle.

In some implementations, the resilient spacer material and structure is not simply adapted to be clamped down into a squished shape of reduced volume and dimensions (like existing spacers). Instead, it is sized, structurally formed, and shaped such that when it is mechanically compressed between the anchors, it compressively expands its dimensions to bulge outward and be wider and/or longer than its uncompressed shape. Thereby, potentially the resilient spacer extrudes to extend beyond the anchor opposing surfaces (typically in a balloon-like fashion) to fill spaces adjacent to and beyond the boundaries of the anchors in response to forces (e.g., compression forces, actuation forces, movement forces, connection forces, etc.) that are available from the system and/or mechanism (e.g., connection system adjusting connection and position between the anchors and spacer). This effect can occur because of the material and structure's typically unitary form (e.g., it is not simply a flexible mesh on a frame) and functionally low compressibility.

In some implementations, when the first, second and third leaflet anchors 213, 215, 217 are drawn to the compliant center body 261 by their respective controlled tethers, the controlled tethers are pulled with a centering force adequate to force the anchors to act as compressive bodies that deform the compliant center body to fill all voids between the anchors, and typically, to protrude past the boundaries of the anchors into the space between the leaflets. In doing so, the leaflets are also drawn further together, which further improves the seal. In some implementations, the anchors are attached to a structural part of the compliant center body directly via the controlled tethers. The structural part can be a central, shaft-shaped catheter around which the spacer forms a pliant periphery.

Advantageously, this compliant center body allows leaflets to function and move relative to one another more normally during valve operation, particularly as compared to devices that do not use a center body, or ones that use a center body that rigidly hold leaflets.

The above-described apparatus and systems provide and/or can be used in one or more methods for treating/repairing a native valve of a subject, the valve having a plurality of leaflets (e.g., a first leaflet and a second leaflet, and in some cases a third leaflet). In some implementations, the method entails providing a treatment or repair device 101 mounted at a distal end of a delivery catheter or implant catheter 111. The methods can be performed on a living animal or on a simulation, such as on a cadaver, cadaver heart, simulator (e.g., with the body parts, heart, tissue, etc. being simulated), etc.

In some implementations, the device 101 includes the first leaflet anchor 113, the second leaflet anchor 115, the first anchor connector 121, and/or the system (e.g., controlled tether system, tether system, coaptation system, actuation system, connection system, etc.). In some implementations, the first anchor connector forms a decouplable/detachable/severable connection between the first leaflet anchor 113 and the delivery or implant catheter 111. In some implementations, the second leaflet anchor is connected to the implant catheter independently of the first anchor connector. In some implementations, the system is actuatable to move the first and second leaflet anchors closer together and/or into contact (e.g., with each other and/or with an optional center body, coaptation element, or spacer), even with the first connection decoupled or detached (e.g., in its decoupled state, detached state, or severed state).

In some implementations, the method includes the steps of delivering the treatment/repair device mounted on the implant catheter to the valve, positioning the first leaflet anchor 113 at the first leaflet, attaching the first leaflet anchor to the first leaflet, decoupling/detaching/severing the first anchor connector 121, positioning the second leaflet anchor 115 at the second leaflet, attaching the second leaflet anchor to the second leaflet, connecting the first and second leaflet anchors, and releasing the device 101 from the implant catheter.

In some implementations, the first anchor connector 121 is decoupled/detached/severed after the first leaflet anchor 113 is attached to the first leaflet. In some implementations, the second leaflet anchor 115 is in turn positioned at the second leaflet after the first anchor connector is decoupled/detached/severed. Once the second leaflet anchor is positioned at the second leaflet, the second leaflet anchor can be attached to the second leaflet.

In some implementations, after the first and second leaflet anchors are attached to the respective first and second leaflets (and the first anchor connector 121 is decoupled/detached/severed), the first and second leaflet anchors are moved closer together and/or into contact (with each other and/or another portion of the device) using the system (e.g., controlled tether system, tether system, coaptation system, connection system, actuation system, control system, etc.). In some implementations, once they are connected and/or moved into a desired relative position, the device 101 is released from the implant catheter.

In some implementations, this method can include the use of the anchor-lock. In some implementations, the device 101 includes the anchor-lock, which has a locking mechanism that locks the relative positions of the first and second leaflet anchors (with respect to one another). In some implementations, after the first and second leaflet anchors are connected (e.g., in contact, connected to each other via contact with another portion or center body, etc.), the first and second leaflet anchors are locked in their relative positions with respect to one another using the anchor-lock.

In some implementations, the system can originally be provided without adequate slack for the second leaflet anchor 115 to be positioned at the second leaflet without interference from the connection to the first leaflet. The method can further include controllably releasing slack on the system. In some implementations, this will generally occur after the step of decoupling/detaching/severing the first anchor connector 121, and before the step of attaching the second leaflet anchor to the second leaflet.

In the case of a valve with three leaflets, the device 101 can include the second anchor connector forming a decouplable/detachable/severable connection between the second leaflet anchor 115 and the implant catheter. In some implementations, a third leaflet anchor is connected to the implant catheter independently of the first and second anchor connectors, and the system is actuatable to move the first, second and third leaflet anchors closer and/or into contact (with each other and/or another portion of the device, e.g., a center body, spacer, edge, surface, etc.) after the first and second decouplable/detachable connections are decoupled/detached/severed.

In some implementations, after the second leaflet anchor 115 is attached to the second leaflet (of the three leaflets),), the second anchor is connector decoupled/detached/severed. In some implementations, the third leaflet anchor is positioned at the third leaflet, and the third leaflet anchor is attached to the third leaflet. In some implementations, in the step of moving, the first, second and third leaflet anchors are moved closer to and/or into contact with one another (or with another portion of the device).

In some implementations of the methods in which the various systems, devices, apparatuses, etc. herein may be used, including without limitation in the above-described method for treating/repairing a native valve of a subject, the various systems, devices, apparatuses, etc. herein can include one or more anchors that are detached from leaflets and/or reattachable to leaflets, and the methods can include detaching one or more anchors from one or more leaflets and/or reattaching one or more anchors to one or more leaflets.

In some implementations, after an anchor connector is decoupled/detached/severed, the system (e.g., controlled tether system, tether system, coaptation system, actuation system, connection system, control system, etc.) can be used to connect the respective leaflet anchor to the implant catheter. In some implementations, the implant catheter can be used to reposition the respective anchor with respect to its leaflet (or another leaflet). In some implementations, the leaflet anchor can then be attached in that new position, and the method may proceed as before.

In some implementations, the method further can include moving the first leaflet anchor and the implant catheter using the system, detaching the first anchor connector from the first location (e.g., detaching from tissue at a first location), and attaching the first leaflet anchor at another location (e.g., a different tissue location).

The methods described above can be performed on a living subject or on a simulation, such as on a cadaver, cadaver heart, simulator (e.g., with the body parts, heart, tissue, etc. being simulated), etc.

In some implementations, each of a plurality of leaflet anchors is consecutively inserted into the subject, some of which (a plurality) are separately pulled (independently from one another) toward and to a center body by distinct controlled tethers. Additional details of these implementations can be drawn from similar aspects of the first (simultaneously inserted, center-coupled) implementation. These consecutively inserted implementations are applicable to two- and three-leaflet valves (as is further described) and can incorporate presently used procedures for repairing three-leaflet valves using only two leaflet anchors.

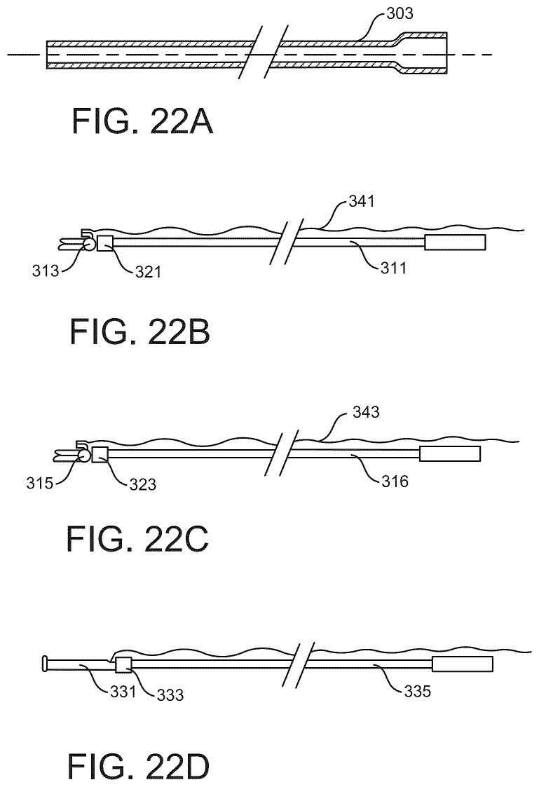

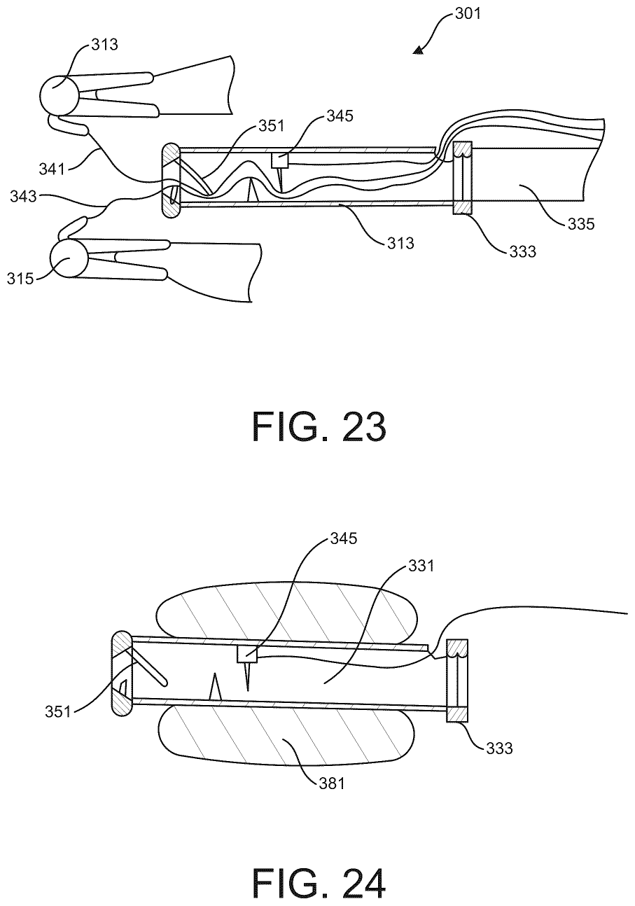

With reference to FIGS. 2-8, 22A, 22B, 22C, 22D, 23, and 24, in some implementations (e.g., in consecutively inserted, center-coupled implementations of a treatment or repair system), a treatment or repair device 301 for use by medical personnel in treating or repairing a subject's native mitral heart valve MV, can be constructed or assembled within the heart (at the location of the valve) using a delivery system. Referring to FIG. 22A, in some implementations, the delivery system is provided with a steering catheter system and/or a steering catheter 303, which can operate similar to other steering catheter systems or steering catheters. Advancing the delivery through the vascular system can involve positioning a distal end of the steering catheter 303 in place upstream of the valve (relative to a blood flow direction) and pointing the distal end of the steering catheter downstream (in the blood flow direction) through the valve.

Referring to FIGS. 22B-22D, in some implementations, the delivery system is further provided with an implant catheter system that includes one or more implant catheters (which can be the same as or similar to other catheters herein; and can be in addition to or in place of a steering catheter). In some implementations, the implant catheter system comprises a plurality of implant catheters. In some implementations, each implant catheter has a catheter distal end that traverses the steering catheter system for implantation of a treatment/repair device, and a catheter proximal end (e.g., a first catheter proximal end, a second catheter proximal end, and a third catheter proximal end) that is user accessible for a user to remotely manipulate and operate the device during implantation.

In some implementations, a first implant catheter 311 of the plurality of implant catheters either starts off within the steering catheter system prior to its insertion into the subject's body or is inserted into a proximal (user) end of the steering catheter system (the proximal end of which extends outside the subject's body) after the distal end of the steering catheter system is positioned upstream of the mitral valve MV. In some implementations, with the steering catheter system inserted in place and the first implant catheter 311 inserted in the steering catheter system, a first catheter distal end of the first implant catheter 311 is extended from within steering catheter system, downstream toward the mitral valve (similar to the implant catheter depicted in FIG. 9).

In some implementations, at its first catheter distal end, the first implant catheter 311 carries a first leaflet anchor 313 of the plurality of leaflet anchors of the device. Each leaflet anchor of the plurality of leaflet anchors is in the form of an actuatable clip with independent actuators and actuation controls such that each anchor can be separately and independently affixed to respective leaflet locations. In some implementations, the leaflet anchors are structurally adapted to affix to a leaflet by clipping onto a leaflet.

In some implementations, each of the plurality of leaflet anchors is provided with and attached to the distal end of an implant catheter (of the one or more implant catheters) by a respective anchor connector (of a plurality of anchor connectors), e.g., the first leaflet anchor 313 is attached to the first implant catheter 311 by a first anchor connector 321, a second leaflet anchor 315 is attached to a second implant catheter 316 by a second anchor connector 323, a third leaflet anchor is attached to a third implant catheter by a third anchor connector, and so on. Additional leaflet anchors may optionally be is attached to implant catheters by respective additional anchor connectors. In some implementations, each anchor connector has a respective catheter connector (e.g., a first catheter connector adapted to connect to the first catheter distal end of the first anchor connector's implant catheter, a second catheter connector adapted to connect to a second catheter distal end of the second anchor connector's implant catheter, and a third catheter connector adapted to connect to a third catheter distal end of the third anchor connector's implant catheter).

In some implementations, the anchor connectors are constructed to actuate between a connected state (e.g., an unsevered state, etc.) and a detached/decoupled state (e.g., a severed state, etc.). In its connected state, the anchor connector's leaflet anchor is constrained not to move in one or more (and typically six) degrees of freedom relative to its delivery/implant catheter (e.g., they are constrained to move in sync with one another). In the anchor connector's detached/decoupled state, the relative movement of the leaflet anchor and its connected implant catheter are no longer constrained by the anchor connector.

In some implementations, each leaflet anchor is also provided with and attached to a respective anchor tether in the form of a controlled tether (of a plurality of controlled tethers), e.g., a first controlled tether 341 is attached to the first leaflet anchor 313, and a second controlled tether 343 is attached to the second leaflet anchor 315. In some implementations, each of the first and second controlled tethers is in the form of a flexible cord (e.g., a suture, wire, line, braid, thread, yarn, etc.).

In some implementations, with each anchor connector connected to the distal end of its delivery or implant catheter, the controlled tether extends from a distal end of the controlled tether at its respective leaflet anchor to a proximal end of the controlled tether at the catheter proximal end of the connected implant catheter. In some implementations, while the implant catheter can be removed after the anchor is connected to a leaflet, each controlled tether fastens its respective anchor to the steering catheter 303 by serially extending through the steering catheter from a respective tether distal end (e.g., a first tether distal end, a second tether distal end, and a third tether distal end) at its respective anchor to a tether proximal end (e.g., a first tether proximal end, a second tether proximal end, and a third tether proximal end) protruding from the proximal (user) end of the steering catheter. In some implementations, each controlled tether is accessible, controllable and actuatable by the user at the proximal end of the steering catheter, either directly or using control mechanisms (e.g., knobs, motors, buttons, switches, sliders, gears, motors, screws, cams, gimbles, etc.) for implantable devices

In some implementations, the treatment/repair device can further be provided with a center body 331 that locks, e.g., a locking center body, and with a center-body connector 333 connecting the center body to a center-body implant catheter 335. In some implementations, the center body forming one or more center-body orifices that are sized and shaped (adapted) to receive the proximal ends of the controlled tethers of the implanted individual leaflet anchors, and through which the first and second controlled tethers 341, 343 extend. In some implementations, with the controlled tethers threaded through the center-body orifice, the center body is slidable along the controlled tethers to their distal ends.

In some implementations, once the center body is inserted into the heart, the controlled tethers can be pulled through the center body from their proximal ends to move the leaflet anchors closer and/or into contact (with each other and/or another portion of the device). The controlled tethers can be configured to pull against edges of the center body, which in turn can help orients the forces to move the leaflet anchors closer and/or into contact.

In some implementations, in use, a first connected assemblage including the first implant catheter 311, the first anchor connector 321, the first leaflet anchor 313 and the first controlled tether 341 is controllably inserted into the steering catheter 303 by the user. Using the first implant catheter, the first leaflet anchor is guided to a leaflet of the valve. In some implementations, the first leaflet anchor 313 is affixed to the valve leaflet, and the first anchor connector 321 is then decoupled/detached/severed. In some implementations, the first implant catheter 311, and any remaining portions of the first anchor connector 321 that are still attached to it, are then withdrawn from the steering catheter 303.

In some implementations, from the time of the insertion, the first controlled tether 341 extends from the first leaflet anchor 313 (which is attached to the leaflet) to the proximal end of the steering catheter outside of the subject's body. After the implantation of the first leaflet anchor is completed, each of the other assemblages of an implant catheter, an anchor connector, an anchor, and a controlled tether are implanted, in that they: 1) are inserted into the steering catheter; 2) affix their anchor to a leaflet; 3) detach/decouple/sever their anchor connector, and 4) remain affixed to the leaflet as the implant catheter is withdrawn from the steering catheter (in a fashion like that with which the first assemblage was done).

Once all of the anchors are serially inserted and attached to the leaflets, a user (outside the subject's body) can feed each of the controlled tethers through a center-body orifice that forms a passage through the locking center body 331, thereby stringing the center body on the full set of controlled tethers. In some implementations, this center-body passage forms an anchor-lock in the form of a cinch-type cable lock 351 that allows the controlled tethers to be pulled through the center body in only one direction, such that the center body can be advanced toward the anchors along the controlled tethers, but not drawn away from the anchors along the controlled tethers. Using its center-body implant catheter 335, the center body 331 can be controllably inserted into the steering catheter 303, and the controlled tethers can be held to prevent them from being drawn fully into the subject's body (thereby causing the center body to advance toward the anchors along the controlled tethers).

As used herein, a cable lock can comprise a device in which a cable (e.g., a tether) is locked in place by a cord-lock element (e.g., a cog, tongue, lock, clamp, set screw, cam, lever, etc.) that squeezes the cord to lock the cord, e.g., to prevent or inhibit relative movement of the cord to the cable lock in one or both of the directions in which the cord extends through the cable lock. This squeezing can occur through various ratcheting or sliding mechanisms, as with cable locks.

In some implementations, using the center-body implant catheter 335, the locking center body 331 can be placed or positioned within the center of the valve, and the controlled tethers can be pulled proximally to move the anchors toward (and to) the location of the center body, and to lock the anchors to the center body, thereby assembling the device in a form that is operable to assist in improved valve operation.

In some implementations, with the anchors fully against and locked onto the center body, a tether severing device (e.g., a tether cutter 345 on the locking center body 331 or center-body implant catheter 335) severs the controlled tethers such that distal segments of the controlled tethers, and their respective anchors, remain cinched and locked in place with respect to the locking center body 331 to maintain connection of the anchors.

In some implementations, the center body and locking mechanism forms the anchor-lock that constrains the relative movement of the first, second and third leaflet anchors after being moved into contact with the center body. The remaining (cut-off) proximal segments of the controlled tethers are then pulled proximally through the steering catheter to remove them from the subject's body.

In some implementations, with all of the proximal segments of the controlled tethers removed, a control can be actuated causing the center-body connector 333 to decouple/detach/sever, releasing the center body, and thereby releasing the assembled device from the center-body implant catheter. In some implementations, the center body 331 and the first and second leaflet anchors 313, 315 are completely freed to translate and rotate in six degrees of freedom with the leaflets (relative to the center-body implant catheter 335 and the rest of the delivery system). In some implementations, the center-body implant catheter 335 and the steering catheter are removed from the subject, and the procedure is finished using typical procedures.

In some implementations, by making the attachment of one or more inserted anchors separable from one another (and the rest of the implant) prior to leaflet attachment, anchors can be attached to leaflets without interference or resistance from other previously attached anchors. The device can therefore be placed without suffering the difficulties caused by leaflets having excessive spacing and movement relative to one another.

In some implementations, the procedure/method can differ from some other implementations in that it provides for greater numbers of anchors and/or anchors with larger form factors to be used through a steering catheter of limited size, and that it further insolates the anchoring procedures from interference by other anchors. It should be appreciated that combinations of these implementations, such as ones in which multiple anchors are inserted on each of multiple consecutively inserted implant catheters, are within the scope herein.

While some implementations are described as connecting three leaflets on a tricuspid valve, other scenarios are also within the scope of the concepts herein. For example, multiple anchors can be attached to one or more leaflets on either a two-leaflet valve or another three-leaflet valve. During the process of implanting, the anchors can be attached alternately between the leaflets (e.g., back, and forth between two or three leaflets), or between two points on a leaflet, and then to another leaflet. Advantageously, this can offer additional structural support to a damaged leaflet at risk of further structural failure, and/or better attachment to a leaflet having characteristics indicating poor anchor-attachment characteristics.

A wide variety of consecutively inserted, center-coupled implementations are within the scope herein, some of which are expressly described herein. To the extent they are usable, these variations are also envisioned for use with other implementations herein. Thus, a treatment/repair system and/or device can include any combination or sub-combination of the features and variations disclosed herein and can exclude some described features.

With reference to FIG. 25, in some consecutively inserted, center-coupled implementations, rather than each controlled tether being unitary (e.g., a unitary anchor tether long enough to extend through the length of the steering catheter), each controlled tether can be formed in two sections: a relatively short anchor-tether section 363 (e.g., three controlled tethers would have a first anchor-tether section, a second anchor-tether section and a third anchor-tether section); and a looped extension-tether section 365 (e.g., a first extension-tether section, a second extension-tether section, and a third extension-tether section).

In some implementations, each anchor-tether section 363 can be permanently attached to its respective anchor 367 at a distal end of the anchor-tether section (e.g., a first distal end, a second distal end, and a third distal end). In some implementations, at a proximal end of the anchor-tether section 363 (e.g., a first proximal end, a second proximal end, and a third proximal end), the anchor-tether section forms an eyelet 369 (e.g., a first eyelet, a second eyelet and a third eyelet) in the form of a slidable eyelet-type restraint through which serves as the primary connection for the extension-tether section 365. This connection is typically made prior to the insertion of the anchor into the subject.

In some implementations, each looped extension-tether section 365 has a proximal, first-end segment 371, a proximal, second-end segment 373 and a distal, intermediate segment 375. For example, a first controlled tether would have a first first-end segment, a first second-end segment, and a first intermediate segment. Likewise, a second controlled tether would have a second first-end segment, a second second-end segment, and a second intermediate segment, and a third controlled tether would have a third first-end segment, a third second-end segment, and a third intermediate segment.

In some implementations, the intermediate segments are intermediate the first-end segments and the second-end segments with respect to (along) the extension-tether sections. In some implementations, when each anchor and its respective two tether sections (the anchor-tether section and extension-tether section) are first implanted, the first-end segment 371 and second-end segment 373 of the extension-tether section 365 are restrained such that they extend out of the proximal end of the steering catheter, while the intermediate segment 375 forms a bight that extends through the eyelet of the anchor-tether section 363 such that the respective anchor can be brought closer and/or into contact by pulling on the first and second-end segments of the extension-tether section 365.

As with some consecutively inserted, center-coupled implementations described previously, when all of the anchors have been affixed to their respective leaflets, the locking center body 331 can be implanted within the center of the valve. In some implementations, the looped extension-tether sections 365 of the controlled tethers can be pulled proximally to move the anchors toward and to the location of the center body, cinching the controlled tethers to lock the respective anchors 367 to the center body, thereby assembling the device in a form that is operable to assist in improved valve operation. In some implementations, the anchor-tether sections 363 are long enough such that, after assembly of the device (by cinching and locking), they extend through the center body and are restrained (locked) by the locking center body to maintain the structure of the implant.

In some implementations, rather than having a tether cutting device that is operated inside of the heart, each of the looped, extension-tether sections can be removed from the subject's body by pulling on the first-end segment of each looped extension-tether section while releasing the second-end segments. As a result, the second-end segments of the extension-tether sections slide up to and out of the eyelets, and then are pulled from the body.

In some consecutively inserted, center-coupled implementations, rather than each anchor having its own associated implant catheter, a single implant catheter (or implant catheter system) is repeatedly used, to consecutively implant each anchor one at a time. To this end, in some implementations, the catheter-connector of the first anchor connector 321 has a quick-release mechanism to decouplably or detachably be first attached to the first implant catheter 311 during an implantation procedure, and then unattached during an implantation procedure, such that the second anchor connector 323 can be attached to the first implant catheter during implantation. For example, a single implant catheter 311 can be repeatedly routed through the steerable catheter 303, attach a leaflet anchor to the native valve leaflet, separate from the leaflet anchor, be pulled out of the steerable catheter, be attached to the next leaflet anchor, and then be routed through the steerable catheter again to deploy the next anchor. This allows a series of anchors to be implanted using a single implant catheter.

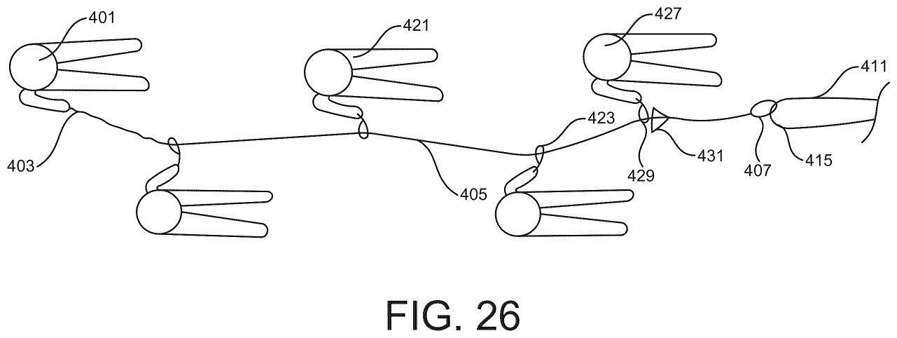

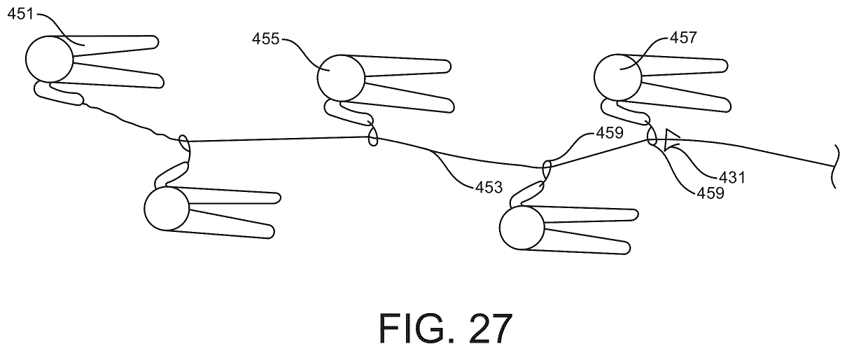

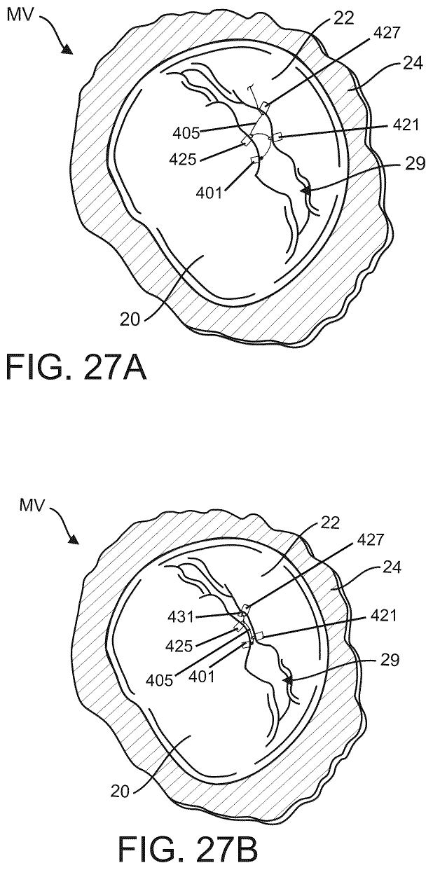

In some implementations, each leaflet anchor has its own tether. In some implementations, the first of a series of leaflet anchors has a tether and subsequent leaflet anchors have a loop that the tether of the first anchor is routed through (see the example of FIGS. 26 and 27). In implementations with a first anchor with a tether that is threaded through loops of subsequent tethers can be implemented with a single implant catheter or multiple implant catheters (e.g., each anchor is connected to its own implant catheter as described above).

Quick-release mechanisms and/or devices are generally capable of being attached and detached in a short enough timeframe to be used in the setting of an implantation of an implant or device without negatively impacting the subject.

In some consecutively inserted, center-coupled implementations, rather than each anchor connector rigidly fastening (restricting) its respective anchor in six degrees of freedom, a reduced number of degrees of freedom may be rigidly restricted. For example, some pivoting and/or translating of the anchor might allow the anchor the ability to resiliently adapt to the changing leaflet position during the ebb and flow of the blood. This reduced restriction may come at a cost of user controllability. User preference and/or case-specific facts might best determine the preferable form of the connectors.

In some consecutively inserted, center-coupled implementations, some or all of the consecutively inserted implant catheters can carry more than one anchor in a manner similar to the first implementation. Advantageously, this limits the number of implant catheter insertions while allowing for a larger number of anchors to be inserted.

In some partially simultaneous or hybrid anchor-delivery implementations, for each consecutive set of simultaneously delivered anchors, more than one anchor is fastened to the catheter by a single controlled tether. In some implementations, either each anchor has one or more respective anchor slidable restraints (e.g., eyelets) through which the single multi-anchor controlled tether serially extends, or each anchor is affixed to a first-end segment of a short length of an anchor-tether section that has an eyelet at its proximal end (through which the single multi-anchor tether serially extends).

In some consecutively inserted, center-coupled implementations, rather than being in the form of a fully flexible cord such as a suture, one or more of the tethers (or tether sections) may be in a less flexible form, such as a mechanism or shaft having its bending and torsional flexibility selectively established to allow more flexibility in some degrees of freedom than in others. While partially limiting the free independent movement of the anchors during the relevant periods of implantation, this form of tether might provide some normalizing force to limit the movement and orientation of the anchors with respect to one another and the center body through the ebb and flow of the blood during implantation. In some implementations, slack describes the extent to which the tether can flexibly allow movement within its moveable degrees of freedom.

In some consecutively inserted, center-coupled implementations, the anchor is a T-shaped toggle anchor. In some implementations, other types of anchors are used (e.g., any of the other anchors described herein, clips, clasps, darts, hooks, staples, screws, coils, etc.)

In some implementations, system/device can include a center body. In some implementations, the center body can include a compliant spacer 381. In some implementations, the compliant spacer can be formed with a unitary, relatively incompressible, pliant periphery (having low compressibility) with adequately low rigidity to be moldable under compression without significant loss of volume. Center bodies and structural details of other center bodies described elsewhere herein and can be incorporated here.

In some implementations, a compliant center body (e.g., its spacer) allows leaflets to function and move relative to one another more normally during valve operation, particularly as compared to devices that do not use a center body, or ones that use a center body that rigidly hold leaflets.

In some end-coupled implementations, a finally implanted plurality of leaflet anchors (e.g., group of anchors) are strung along a single, implant controlled tether (a first controlled tether) formed by one or more lengths of tether running along a single path that has a distal leaflet anchor at a distal end of the implant-controlled tether (which might be the first leaflet anchor that is implanted). In some implementations, the group of anchors includes one or more (e.g., a plurality of) additional leaflet anchors slidably connected to the implant-controlled tether proximal to the distal leaflet anchor. This can be accomplished in a variety of different ways. In some implementations, the distal leaflet anchor has a tether and subsequent leaflet anchors have a loop that the tether of the distal anchor is routed through. This example with a distal anchor with a tether that is threaded through loops of subsequent tethers can be implemented with a single implant catheter or multiple implant catheters as described above. In some implementations, the additional leaflet anchors include a proximal leaflet anchor (the proximal leaflet anchor also being referred to as a second leaflet anchor), and an intermediate leaflet anchor (possibly being one of one or more intermediate leaflet anchors) intermediate the distal and proximal leaflet anchors.

In other words, in the group of anchors being formed by the distal leaflet anchor and the additional leaflet anchors, the proximal leaflet anchor can be at a proximal end of the group of anchors, and the distal leaflet anchor can be at a distal end of the group of anchors, with one or more intermediate leaflet anchors, including a third intermediate leaflet anchor, a fourth intermediate leaflet anchor, and so on, being intermediate the distal and proximal leaflet anchors. In some implementations, each leaflet anchor is provided with a respective leaflet-attachment mechanism (e.g., a first, distal leaflet-attachment mechanism, a third, intermediate leaflet-attachment mechanism and a second, proximal leaflet-attachment mechanism).

In some implementations, an anchor-lock is included that locks onto the implant-controlled tether proximate to the proximal leaflet anchor, constraining any proximal movement of the proximal leaflet anchor (proximally) up the implant-controlled tether. To this end, the anchor-lock can include an end-body that is integral with the proximal leaflet anchor, or it may form a separate end-body that is connected to the implant catheter using an end-body connector. In some implementations, it is configured to apply a distal force to the proximal leaflet anchor, such as by being positioned proximally to the proximal leaflet anchor or by otherwise holding the proximal leaflet anchor from moving distally. In some implementations, the intermediate leaflet anchors are slidably strung on the implant-controlled tether intermediate the distal and proximal leaflet anchors, and are held on the implant-controlled tether by the distal leaflet anchor and the end-body.

In some implementations, the group of leaflet anchors are implanted using a delivery system including an implant catheter having a catheter proximal end and a catheter distal end. In some implementations, the group of leaflet anchors can be separably connected to the implant catheter using one or more anchor connectors (e.g., a distal anchor connector forms a distal anchor detachable/decouplable/severable connection, an intermediate anchor connector forms an intermediate anchor detachable/decouplable/severable connection, and a proximal anchor connector forms a proximal anchor detachable/decouplable/severable connection), with the respective connections being severed after their respective leaflet anchors are attached to leaflets.

In some implementations, the one or more anchor connectors can be in the form of a connector cartridge that consecutively detaches/decouples/severs its connection to a first-implanted through a last-implanted leaflet anchor, which may or may not be the distal and proximal leaflet anchors, respectively.

With all of the leaflet anchors (of the group of anchors) attached to leaflets, in some implementations, the implant-controlled tether can be pulled proximally through an end-body orifice formed by (on) the anchor-lock end-body to draw the distal leaflet anchor toward the proximal leaflet anchor, connecting (e.g., bringing into contact, connecting with another portion in between, bringing into contact with another portion, etc.) the distal and proximal leaflet anchors, and any intermediate leaflet anchors that they draw together in the process. With the distal and proximal leaflet anchors moved into position, in some implementations, the anchor-lock locks onto the implant-controlled tether, constraining the distal and proximal leaflet anchors (e.g., the first and second leaflet anchors) from moving apart from one another with respect to the implant-controlled tether. Optionally, the anchor-lock can be a one-way cinching mechanism that automatically locks on as it moves distally along the implant-controlled tether. With the distal and proximal leaflet anchors fully connected and the anchor-lock locked onto the implant-controlled tether, in some implementations, the full group of anchors is thereby connected and end-coupled in place along the implant-controlled tether. The segment of the implant-controlled tether that extends proximally from the anchor-lock may then be separated (e.g., cut or otherwise disconnected) from the remaining anchor-connected portion of the implant-controlled tether (e.g., the distal portion) and removed from the subject.

In some implementations, end-coupled anchors can be constrained together consecutively along a single implant-tether pathway between two longitudinal-ends of a segment of the implant-controlled tether. In some implementations, the implant-tether segment forming the path can be made of multiple lengths of tether material connected and/or looped, serially and/or in parallel, along the implant-tether pathway between longitudinal ends defined by the proximal and distal leaflet anchors. The terms ‘distal’ and ‘proximal’ anchor may be used in reference to the anchor's position along the implant-controlled tether, and not the location of the implanted anchor within the heart. The terms ‘first-implanted’ and ‘last-implanted’ refer to the chronological order in which the anchors are attached to the leaflets, which may be or may not be the sequence order in which they are positioned along the tether or mounted on the implant catheter.

In some implementations, the implant-controlled tether includes two connected implant-tether sections, a looped, extension-tether section that forms the proximal portion of the implant-controlled tether, and a distal anchor-tether section that forms the distal portion of the implant-controlled tether (and that is affixed to the distal leaflet anchor). In some implementations, the extension-tether section extends between an extension-tether proximal-end segment at a catheter proximal end of an implant catheter of the delivery system, and an extension-tether distal-end segment connected to a proximal end of the distal anchor-tether section.

In some implementations, in assembling and connecting the implant, no pair of intermediate leaflet anchors along the implant-controlled tether are directly and separately moved towards an intermediate position by the anchor-lock alone. Instead, the entire force is exerted at the location of the proximal and distal leaflet anchors, with the distal leaflet anchor being proximally pulled by the distal end of the distal anchor-tether section, and with the proximal leaflet anchor being restrained and/or distally pushed by the end-body supported on the implant catheter. It should be noted that the intermediate leaflet anchors may nevertheless be moved toward one another by the natural rhythm and forces of the leaflets, as each leaflet will exert forces on its respective leaflet anchor or anchors.

In some implementations, the various systems, apparatuses, devices, etc. herein can be configured such that after decoupling an anchor, center body, other component from the delivery or implant catheter, then the delivery or implant catheter can be coupled or recoupled to the anchor, center body, or other component. This can be done using a tether or similar device that allows freedom of movement in the decoupled state, but can be used (e.g., tensioned, etc.) to bring the delivery/implant catheter back into contact with the anchor, center body, or other component. The configuration can be similar to the tether and control systems and mechanisms described herein with respect to bringing the anchors and/or center body together. In some implementations, after recoupling, the delivery or implant catheter can be used to (1) detach the anchor, center body, or other component from a first location, and/or (2) reposition and/or attach the anchor, center body, or other component to a second location (different from the first location) (e.g., to a different tissue location).

Where not specifically described and not functionally precluded, additional details and features of other implementations herein may be included here.

With reference to FIG. 26, prior to implantation, end-coupled implementations can include a plurality of leaflet anchors including a distal leaflet anchor 401 integrally attached to a distal end 403 of an implant-controlled tether. In some implementations, the implant-controlled tether includes two sections, the first section being a distal anchor-tether section 405. The distal leaflet anchor 401 can be a first-implanted leaflet anchor. In some implementations, at its proximal end, the distal anchor-tether section 405 forms a distal anchor-tether eyelet 407, and at a distal end the distal anchor-tether section 405 can be attached to the distal leaflet anchor. In some implementations, the distal anchor-tether section is long enough to extend across the full length of the locked leaflet anchors when the locking operation is complete.

In some implementations, the second section of the implant-controlled tether is a looped, extension-tether section 411 having a first-end segment and a second-end segment, both of which extend out of the proximal end of the delivery system. In some implementations, the implant-controlled tether also includes a longitudinal intermediate segment 415. In some implementations, the intermediate segment 415 of the extension-tether section loops through and extends around the distal anchor-tether eyelet 407 of the distal anchor-tether section 405. This looped configuration provides for the extension-tether section to be removed by pulling on one of its end segments after the locking operation is complete.

In addition to the distal leaflet anchor 401, the plurality of anchors can include one or more additional anchors. In some implementations, as depicted, the plurality of anchors can include four additional anchors, including a (second) proximal leaflet anchor 427, which can be a last-implanted leaflet anchor. Intermediate the distal and proximal leaflet anchors are a third, fourth and fifth leaflet anchor, each of which are intermediate leaflet anchors 421, and each of which may be implanted in their order from the distal leaflet anchor 401 to the proximal leaflet anchor 427. In some implementations, each additional anchor has a respective anchor-tether section, including three intermediate anchor-tether sections 423 and a proximal anchor-tether section 429 (e.g., a second anchor-tether section). The additional anchor-tether sections form eyelets through which the implant-controlled tether (e.g., the combined, extension-tether section 411 and distal anchor-tether section 405) is threaded, providing for the entire group of anchors to be tethered via the implant-controlled tether. In a minimum 2-anchor case, there are no intermediate leaflet anchors 421 or intermediate anchor-tether sections 423.

In some implementations, chronologically after the distal leaflet anchor 401 is attached to its respective leaflet and detached/decoupled/severed from a catheter distal end of an implant catheter of the implant catheter system, for the additional anchors (e.g., the intermediate leaflet anchors 421 and/or the proximal leaflet anchor 427), the additional anchor-tether sections (first the intermediate anchor-tether sections 423 and then the proximal anchor-tether section 429) of the additional anchors are guided to the respective heart valve leaflet by the implant catheter, where the additional anchor is then attached to the leaflet. One by one each additional anchor can be consecutively attached, with each respective additional anchor-tether section being strung along the extension-tether section 411. When all anchors of the group of anchors have been affixed to leaflets, the result is a series of consecutively placed leaflet anchors that are serially located in consecutive order along a single, implant controlled tether in a stitched configuration.

In some implementations, when the group of anchors are all attached to their respective leaflets, an end-body 431 (being similar in form and cinching function to the locking center bodies of prior implementations and variations), which is separably connected to the implant catheter with an end-body connector, is advanced along the implant controlled tether by proximally pulling on the extension-tether section such that the implant controlled tether is pulled back through the end-body while using the implant catheter to hold the end-body. In some implementations, the distal anchor-tether section 405 is pulled through the end-body causing the end-body to be cinched up onto the distal anchor-tether section, causing the distal leaflet anchor 401 and the proximal leaflet anchor 427 to be mutually pulled toward one another by the distal anchor-tether section. This pulling on the extension-tether section 411 relative to the end-body's implant catheter leads to the completion of the implant assembly. When this occurs, the intermediate leaflet anchors 421 naturally adjust their positions based on their relative locations on the various leaflets, and on the tension and pathway over which the distal anchor-tether section 405 extends.

In some implementations, the extension-tether section 411 is then removed from the subject's body while leaving the implanted device in place, by pulling on the first-end segment of the extension-tether section while releasing the second-end segment so as to be pulled through and around the path of the extension-tether section's loop.

In some implementations, the systems, devices, methods, etc. herein comprise a stitching of the leaflets, wherein each stich is a planned distance from the previous stich. Part of the stitching can extend back and forth between multiple leaflets, while other parts can progress across a single leaflet. The stiches can be placed along a leaflet at a predetermined spacing by selecting the attachment locations, and/or by using spacers between consecutive anchors.

FIGS. 27A and 27B illustrate one of the many potential uses for the examples of FIGS. 26 and 27. Referring to FIG. 27A, a distal leaflet anchor 401 is attached to a leaflet 20. Then, a leaflet anchor 421 is routed along the tether 405 and attached to a leaflet 22. Then a leaflet anchor 425 is routed along the tether 405 and attached to the leaflet 22. Then a leaflet anchor 427 is routed along the tether 405 and attached to the leaflet 20. This sequence forms a back-and-forth stitch pattern. Any number of anchors can be used. While the mitral valve is illustrated, the sequence illustrated by FIGS. 27A and 27B can be applied to any valve, such as a tricuspid valve. Further, the example illustrated by FIGS. 27A and 27B can be used in a tricuspid valve in the same or similar way to the example illustrated by FIGS. 16-20.

Referring to FIG. 27B, in some implementations, when the group of anchors are all attached to the, an end-body 431 or lock (being similar in form and cinching function to the locking center bodies of prior examples and variations), is advanced along the tether 405 by proximally pulling on the tether. The tether is pulled back through the end-body to draw the leaflet anchors 401, 421, 425, 427 together. As a result, the anchors 401, 421, 425, 427 draw the leaflets 20, 22 together to reduce regurgitation through the native valve in the area of the anchors. The intermediate leaflet anchors 421, 425 naturally adjust their positions on the tether 405. The end body 431 or lock is fixed on the tether 405 to secure the relative positions of the leaflet anchors 401, 421, 425, 427 and complete the native valve repair. The tether 405 can be cut off at the end body 431 and be removed.

It should be noted that this example can provide for a stitching of the leaflets, wherein each stitch is a planned distance from the previous stitch. Part of the stitching can extend back and forth between multiple leaflets, while other parts can progress across a single leaflet. The stitches can be placed along a leaflet at a predetermined spacing by selecting the attachment locations, and/or by using spacers between consecutive anchors.

With reference to FIG. 27, in some end-coupled implementations, a distal leaflet anchor 451 is permanently affixed to a single, unitary controlled tether 453 (e.g., it is unitary in that it is not made of separate sections) that is long enough to extend through the length of the steering catheter from its distal to its proximal end. In some implementations (similar to other implementations described previously), intermediate leaflet anchors 455 and a proximal leaflet anchor 457 can have anchor-tether sections 459 that are strung on the unitary controlled tether 453. In some implementations, a tether cutting device in an implant catheter is employed to cut off and remove most of the long, unitary controlled tether 453 from the implant after the implant is fully assembled and the anchors are cinched together by the end-body 431.

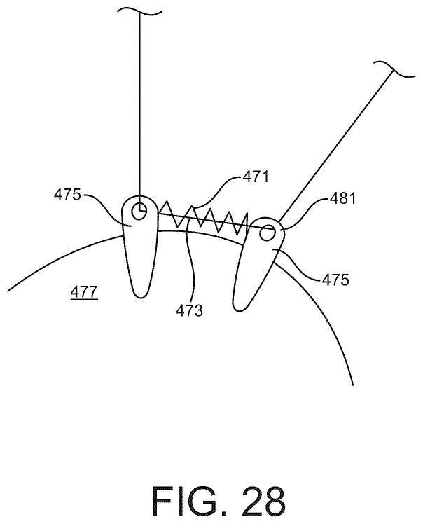

With reference to FIG. 28, in some end-coupled implementations, one or more (spring-type) spring spacers 471 are placed on the segments of implant-controlled tether 473 extending between some or all consecutive pairs of leaflet anchors 475 that are attached to leaflets 477 (e.g., pairs of leaflet anchors that occur consecutively along the implant-controlled tether). The one or more spring spacers 471 are placed by feeding them up the extension-tether section, each being fed between a respective consecutive pair of anchors that the spring spacer will separate. These spring spacers can help maintain anchors at preferred distances from one another. Structurally, these spring spacers 471 can be formed as one of the forms of spacer described above, or they can be of other configurations, such as an accordion-folded pledget, wherein the implant-controlled tether 473 penetrates and extends through each accordion fold (as depicted).

With reference to FIG. 28, in some implementations, rather than the additional leaflet anchors having anchor-tether sections that form eyelets through which an extension-tether section slidably extends, leaflet anchors 475 can include structurally integral orifices making slidable restraints 481 (e.g., a proximal anchor slidable restraint and an intermediate anchor slidable restraint) through which the segments of implant-controlled tether 473 slidably extend.

In some end-coupled implementations, the anchors are configured as T-shaped toggle anchors. This type of anchor may provide for enhanced support for lateral loads occurring between consecutive anchors, and particularly on consecutive anchors attached to different leaflets. Other types of anchors are also possible (e.g., anchors described elsewhere herein).

Additional features, modifications, delivery systems, and methods for using the devices and delivery systems are disclosed by Patent Cooperation Treaty International Application No. PCT/US2019/012707 (International Publication No. WO 2019139904). Any combination or sub-combination of the features disclosed by the present application can be combined with any combination or sub-combination of the features disclosed by Patent Cooperation Treaty International Application No. PCT/US2019/012707 (International Publication No. WO 2019139904). Patent Cooperation Treaty International Application No. PCT/US2019/012707 (International Publication No. WO 2019139904) is incorporated herein by reference in its entirety for all purposes.

Clasps or leaflet gripping devices disclosed herein can take a wide variety of different forms, such as those disclosed by Patent Cooperation Treaty International Application No. PCT/US2018/028171 (International Publication No. WO 2018195201). Combinations and sub-combinations of features in the present application can be combined with the features disclosed by Patent Cooperation Treaty International Application No. PCT/US2018/028171 (International Publication No. WO 2018195201), which is incorporated herein by reference in its entirety for all purposes.

Any of the various systems, devices, apparatuses, etc. in this disclosure can be sterilized (e.g., with heat, radiation, ethylene oxide, hydrogen peroxide, etc.) to ensure they are safe for use with subjects, and the methods herein can comprise or consist of sterilization of one or more systems, devices, apparatuses, etc. herein (e.g., with heat, radiation, ethylene oxide, hydrogen peroxide, etc.).

While various inventive aspects, concepts and features of the disclosures may be described and illustrated herein as embodied in combination in the examples herein, these various aspects, concepts, and features may be used in many alternative examples, either individually or in various combinations and sub-combinations thereof. Unless expressly excluded herein all such combinations and sub-combinations are intended to be within the scope of the present application. Still further, while various alternative examples as to the various aspects, concepts, and features of the disclosures-such as alternative materials, structures, configurations, methods, devices, and components, alternatives as to form, fit, and function, and so on—may be described herein, such descriptions are not intended to be a complete or exhaustive list of available alternative examples, whether presently known or later developed. Those skilled in the art may readily adopt one or more of the inventive aspects, concepts, or features into additional examples and uses within the scope of the present application even if such examples are not expressly disclosed herein.

Additionally, even though some features, concepts, or aspects of the disclosures may be described herein as being a preferred arrangement or method, such description is not intended to suggest that such feature is required or necessary unless expressly so stated. Still further, example or representative values and ranges may be included to assist in understanding the present application, however, such values and ranges are not to be construed in a limiting sense and are intended to be critical values or ranges only if so expressly stated.

Moreover, while various aspects, features and concepts may be expressly identified herein as being inventive or forming part of a disclosure, such identification is not intended to be exclusive, but rather there may be inventive aspects, concepts, and features that are fully described herein without being expressly identified as such or as part of a specific disclosure, the disclosures instead being set forth in the appended claims. Descriptions of example methods or processes are not limited to inclusion of all steps as being required in all cases, nor is the order that the steps are presented to be construed as required or necessary unless expressly so stated. The treatment techniques, methods, steps, etc. described or suggested herein or in references incorporated herein can be performed on a living animal or on a non-living simulation, such as on a cadaver, cadaver heart, anthropomorphic ghost, simulator (e.g., with the body parts, tissue, etc. being simulated), etc. The words used in the claims have their full ordinary meanings and are not limited in any way by the description of the examples in the specification.

While forms of the invention have been illustrated and described, it will be apparent that various modifications can be made without departing from the spirit and scope of the invention. Thus, although the invention has been described in detail with reference only to the preferred embodiments, those having ordinary skill in the art will appreciate that various modifications can be made without departing from the scope of the invention. Accordingly, the invention is not intended to be limited by the above discussion, and it is defined with reference to the following claims.

What is claimed:

1. A valve repair device comprising;
a first leaflet anchor comprising a first arm and a second arm, wherein the first arm and the second arm of the first leaflet anchor are configured to be closed to attach the first anchor to a first native heart valve leaflet;
a second leaflet anchor comprising a first arm and a second arm, wherein the first arm and the second arm of the second leaflet anchor are configured to be closed to attach the second anchor to a second native heart valve leaflet;
a spacer disposed between the first leaflet anchor and the second leaflet anchor;
a first tether that extends between the first leaflet anchor and the spacer;
a second tether that extends between the second leaflet anchor and the spacer;
wherein the first tether is configured to be slackened to allow the spacer to move away from the first leaflet anchor;
wherein the second tether is configured to be slackened to allow the spacer to move away from the second leaflet anchor;
wherein the first tether is configured to be tightened to pull the spacer and the first leaflet anchor together; and
wherein the second tether is configured to be tightened to pull the spacer and the second leaflet anchor together.

2. The valve repair device of claim 1 further comprising:
a third leaflet anchor configured to be attached to a third native heart valve leaflet;
a third tether that extends between the third leaflet anchor and the spacer;
wherein the third tether is configured to be slackened to allow the spacer to move away from the third leaflet anchor; and
wherein the third tether is configured to be tightened to pull the spacer and the third leaflet anchor together.

3. The valve repair device of claim 1 wherein the first arm and the second arm of the first leaflet anchor are connected together at a closed end of the first leaflet anchor.

4. The valve repair device of claim 3 wherein the first leaflet anchor is configured to snap to a closed configuration and the first leaflet anchor is configured to snap to an open configuration.

5. The valve repair device of claim 4 wherein the first arm and the second arm are formed from a single piece of curved or bent material.

6. The valve repair device of claim 1 further comprising an anchor lock disposed in the spacer that is configured to lock a position of the first tether.

7. The valve repair device of claim 6 wherein the anchor lock is configured to lock a position of the second tether.

8. The valve repair device of claim 1 further comprising a tether cutter disposed in the spacer, wherein the tether cutter is configured to cut the first and second tethers.

9. The valve repair device of claim 1 wherein the spacer comprises a spring spacer.

10. The valve repair device of claim 9 wherein the spring spacer is configured to maintain the first leaflet anchor and the second leaflet anchor at a predetermined distance from one another.

11. A valve repair system comprising:
a delivery catheter;
a first tether extending through the delivery catheter;
a second tether extending through the delivery catheter;
a valve repair device coupled to the delivery catheter, the valve repair device comprising:
a first leaflet anchor comprising a first arm and a second arm, wherein the first arm and the second arm of the first leaflet anchor are configured to be closed to attach the first anchor to a first native heart valve leaflet;
a second leaflet anchor comprising a first arm and a second arm, wherein the first arm and the second arm of the second leaflet anchor are configured to be closed to attach the second anchor to a second native heart valve leaflet;
a spacer disposed between the first leaflet anchor and the second leaflet anchor;
wherein the first tether extends between the first leaflet anchor and the spacer;
wherein the second tether extends between the second leaflet anchor and the spacer;
wherein the first tether is configured to be slackened to allow the spacer to move away from the first leaflet anchor;
wherein the second tether is configured to be slackened to allow the spacer to move away from the second leaflet anchor;
wherein the first tether is configured to be tightened to pull the spacer and the first leaflet anchor together; and
wherein the second tether is configured to be tightened to pull the spacer and the second leaflet anchor together.

12. The valve repair system of claim 11 further comprising:
a third leaflet anchor configured to be attached to a third native heart valve leaflet;
a third tether that extends between the third leaflet anchor and the spacer;
wherein the third tether is configured to be slackened to allow the spacer to move away from the third leaflet anchor; and
wherein the third tether is configured to be tightened to pull the spacer and the third leaflet anchor together.

13. The valve repair system of claim 11 wherein the first arm and the second arm of the first leaflet anchor are connected together at a closed end of the first leaflet anchor.

14. The valve repair system of claim 13 wherein the first leaflet anchor is configured to snap to a closed configuration and the first leaflet anchor is configured to snap to an open configuration.

15. The valve repair system of claim 11 further comprising an anchor lock disposed in the spacer that is configured to lock a position of the first tether.

16. The valve repair system of claim 15 wherein the anchor lock is configured to lock a position of the second tether.

17. The valve repair system of claim 11 further comprising a tether cutter disposed in the spacer, wherein the tether cutter is configured to cut the first and second tethers.

18. The valve repair system of claim 11 wherein the spacer comprises a spring spacer.

19. The valve repair system of claim 18 wherein the spring spacer is configured to maintain the first leaflet anchor and the second leaflet anchor at a predetermined distance from one another.

20. A method of repairing a heart valve comprising;
attaching a first leaflet anchor to a first native heart valve leaflet;
moving a spacer and a second leaflet anchor away from the first leaflet anchor;
attaching the second leaflet anchor to a second native heart valve leaflet; and
pulling a tether to pull the first leaflet anchor and the spacer together.
